SEQUENCE LISTING (73 sequences)

SEQ ID NO: 1 - DNA, 57 bp, synthetic construct
  source 1..57: mol_type = other DNA
gccaccaacttctccctgctgaagcaggccggcgacgtggaggagaaccccggcccc

SEQ ID NO: 2 - DNA, 63 bp, synthetic construct
  source 1..63: mol_type = other DNA
ggaagcggagagggcagaggaagtctgctaacatgcggtgacgtcgaggagaatcctggacct

SEQ ID NO: 3 - protein, 21 aa, synthetic construct
GSGEGRGSLLTCGDVEENPGP

SEQ ID NO: 4 - protein, 22 aa, synthetic construct
GSGATNFSLLKQAGDVEENPGP

SEQ ID NO: 5 - DNA, 20 bp, synthetic construct
  source 1..20: mol_type = other DNA
acaccaggagacttggaacg

SEQ ID NO: 6 - DNA, 48 bp, synthetic construct
  source 1..48: mol_type = other DNA
gcctgcaagaactggttcagcagcctgagccacttcgtgatccacctg

SEQ ID NO: 7 - DNA, 12 bp, synthetic construct
  source 1..12: mol_type = other DNA
cgaaaaagaaga

SEQ ID NO: 8 - DNA, 20 bp, synthetic construct
  source 1..20: mol_type = other DNA
ggatagcacagcctggatag

SEQ ID NO: 9 - DNA, 55 bp, synthetic construct
  source 1..55: mol_type = other DNA
gataggcacctattggtcttactgacatccactttgcctttctctccacaggtgt

SEQ ID NO: 10 - DNA, 361 bp, Homo sapiens
  source 1..361: mol_type = other DNA
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcg

SEQ ID NO: 11 - DNA, 169 bp, Homo sapiens
  source 1..169: mol_type = other DNA
gtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccag

SEQ ID NO: 12 - DNA, 166 bp, Homo sapiens
  source 1..166: mol_type = other DNA
gtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggag

SEQ ID NO: 13 - DNA, 240 bp, Homo sapiens
  source 1..240: mol_type = other DNA
gccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcag

SEQ ID NO: 14 - DNA, 108 bp, Homo sapiens
  source 1..108: mol_type = other DNA
ttccggaactgcatgctcaccaccatctgctgcggcaagaacccactgggtgacgatgaggcctctgctaccgtgtccaagacggagacgagccaggtggcaccagca

SEQ ID NO: 15 - DNA, 555 bp, synthetic construct
  source 1..555: mol_type = other DNA
taagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 16 - DNA, 215 bp, synthetic construct
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 17 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 18 - DNA, 72 bp, synthetic construct
  source 1..72: mol_type = other DNA
tgtagttaatgattaacccgccatgctacttatctacgtagccatgctctaggaagatcggaattcactagt

SEQ ID NO: 19 - DNA, 20 bp, synthetic construct
  source 1..20: mol_type = other DNA
taataaataataaataataa

SEQ ID NO: 20 - DNA, 50 bp, synthetic construct
  source 1..50: mol_type = other DNA
tgacaaactgtacatgccgttaactgtaattttgcgtgatttttttgtag

SEQ ID NO: 21 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 22 - DNA, 96 bp, synthetic construct
  source 1..96: mol_type = other DNA
aagggcgatatccatcacactggcggcgaattcccgattaggaaagggcgaattctgcagatactagtaacggccgccagtgtgctggaattcagg

SEQ ID NO: 23 - DNA, 377 bp, synthetic construct
  source 1..377: mol_type = other DNA
ctgacctcgagtttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggactcgcgcgagtcgaggaggttttagagctagaaatagcaagttaaaataaggctagtccgttatcaacttgaaaaagtggcaccgagtcggtgcttttttgttttagagctagaaatagcaag

SEQ ID NO: 24 - DNA, 20 bp, synthetic construct
  source 1..20: mol_type = other DNA
gactcgcgcgagtcgaggag

SEQ ID NO: 25 - DNA, 186 bp, synthetic construct
  source 1..186: mol_type = other DNA
ctcgagcacctgaattctgcagatatccatcacactggcggcatccatcacactggcggcgaattcccgattaggaaagggcgaattctgcagatatccatcacactggcggcgaattcccgattaggatcttcctagagcatggctacgtagataagtagcatggcgggttaatcattaactaca

SEQ ID NO: 26 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 27 - DNA, 3765 bp, synthetic construct
  source 1..3765: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcactagtacaccaggagacttggaacgcgggataggcacctattggtcttactgacatccactttgcctttctctccacaggtgttaataaataataaataataatgacaaactgtacatgccgttaactgtaattttgcgtgatttttttgtagatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggcaccagcaggaagcggagagggcagaggaagtctgctaacatgcggtgacgtcgaggagaatcctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaacaccaggagacttggaacgcggaagggcgatatccatcacactggcggcgaattcccgattaggaaagggcgaattctgcagatactagtaacggccgccagtgtgctggaattcaggctgacctcgagtttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggactcgcgcgagtcgaggaggttttagagctagaaatagcaagttaaaataaggctagtccgttatcaacttgaaaaagtggcaccgagtcggtgcttttttgttttagagctagaaatagcaagctcgagcacctgaattctgcagatatccatcacactggcggcatccatcacactggcggcgaattcccgattaggaaagggcgaattctgcagatatccatcacactggcggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 28 - DNA, 377 bp, synthetic construct
  source 1..377: mol_type = other DNA
ctgacctcgagtttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaccaggagacttggaacggttttagagctagaaatagcaagttaaaataaggctagtccgttatcaacttgaaaaagtggcaccgagtcggtgcttttttgttttagagctagaaatagcaag

SEQ ID NO: 29 - DNA, 186 bp, synthetic construct
  source 1..186: mol_type = other DNA
ctcgagcacctgaattctgcagatatccatcacactggcggcatccatcacactggcggcgaattcccgattaggaaagggcgaattctgcagatatccatcacactggcggcgaattcccgataaggatcttcctagagcatggctacgtagataagtagcatggcgggttaatcattaactaca

SEQ ID NO: 30 - DNA, 3765 bp, synthetic construct
  source 1..3765: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcactagtacaccaggagacttggaacgcgggataggcacctattggtcttactgacatccactttgcctttctctccacaggtgttaataaataataaataataatgacaaactgtacatgccgttaactgtaattttgcgtgatttttttgtagatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggcaccagcaggaagcggagagggcagaggaagtctgctaacatgcggtgacgtcgaggagaatcctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaacaccaggagacttggaacgcggaagggcgatatccatcacactggcggcgaattcccgattaggaaagggcgaattctgcagatactagtaacggccgccagtgtgctggaattcaggctgacctcgagtttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaccaggagacttggaacggttttagagctagaaatagcaagttaaaataaggctagtccgttatcaacttgaaaaagtggcaccgagtcggtgcttttttgttttagagctagaaatagcaagctcgagcacctgaattctgcagatatccatcacactggcggcatccatcacactggcggcgaattcccgattaggaaagggcgaattctgcagatatccatcacactggcggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 31 - DNA, 3659 bp, synthetic construct
  source 1..3659: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcactagtacaccaggagacttggaacgcgggataggcacctattggtcttactgacatccactttgcctttctctccacaggtgttaataaataataaataataagccaccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggcaccagcaggaagcggagagggcagaggaagtctgctaacatgcggtgacgtcgaggagaatcctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaacaccaggagacttggaacgcggactagtaacggccgccagtgtgctggaattcaggctgacctcgagtttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggactcgcgcgagtcgaggaggttttagagctagaaatagcaagttaaaataaggctagtccgttatcaacttgaaaaagtggcaccgagtcggtgcttttttgttttagagctagaaatagcaagctcgagcacctgaattctgcagatatccatcacactggcggcatccatcacactggcggcgaattcccgattaggaaagggcgaattctgcagatatccatcacactggcggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 32 - DNA, 3908 bp, synthetic construct
  source 1..3908: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcactagtcaattggcggccgcacaccaggagacttggaacgcgggataggcacctattggtcttactgacatccactttgcctttctctccacaggtgtagactataaggaccacgacggagactacaaggatcatgatattgattacaaagacgatgacgataaggcctgcaagaactggttcagcagcctgagccacttcgtgatccacctgcgaaaaagaagaggaagcggagccaccaacttctccctgctgaagcaggccggcgacgtggaggagaaccccggccccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggcaccagcaggaagcggagagggcagaggaagtctgctaacatgcggtgacgtcgaggagaatcctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaacaccaggagacttggaacgcggaagggcgatatccatcacactggcggcgaattcccgattaggaaagggcgaattctgcagatggtaccactagtaacggccgccagtgtgctggaattcaggctgacctcgagtttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaccaggagacttggaacggttttagagctagaaatagcaagttaaaataaggctagtccgttatcaacttgaaaaagtggcaccgagtcggtgcttttttgttttagagctagaaatagcaagctcgagcacctgaattctgcagatatccatcacactggcggcatccatcacactggcggcgaattcccgattaggaaagggcgaattctgcagatatccatcacactggcggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 33 - DNA, 67 bp, synthetic construct
  source 1..67: mol_type = other DNA
agactataaggaccacgacggagactacaaggatcatgatattgattacaaagacgatgacgataag

SEQ ID NO: 34 - DNA, 3908 bp, synthetic construct
  source 1..3908: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcactagtcaattggcggccgcacaccaggagacttggaacgcgggataggcacctattggtcttactgacatccactttgcctttctctccacaggtgtagactataaggaccacgacggagactacaaggatcatgatattgattacaaagacgatgacgataaggcctgcaagaactggttcagcagcctgagccacttcgtgatccacctgcgaaaaagaagaggaagcggagccaccaacttctccctgctgaagcaggccggcgacgtggaggagaaccccggccccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggcaccagcaggaagcggagagggcagaggaagtctgctaacatgcggtgacgtcgaggagaatcctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaacaccaggagacttggaacgcggaagggcgatatccatcacactggcggcgaattcccgattaggaaagggcgaattctgcagatggtaccactagtaacggccgccagtgtgctggaattcaggctgacctcgagtttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggactcgcgcgagtcgaggaggttttagagctagaaatagcaagttaaaataaggctagtccgttatcaacttgaaaaagtggcaccgagtcggtgcttttttgttttagagctagaaatagcaagctcgagcacctgaattctgcagatatccatcacactggcggcatccatcacactggcggcgaattcccgattaggaaagggcgaattctgcagatatccatcacactggcggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 35 - protein, 23 aa, synthetic construct
GSGQCTNYALLKLAGDVESNPGP

SEQ ID NO: 36 - protein, 25 aa, synthetic construct
GSGVKQTLNFDLLKLAGDVESNPGP

SEQ ID NO: 37 - protein, 35 aa, synthetic construct
SLISLPLPTRVKFSSLLLIRIMKIITMTFPKKLRS

SEQ ID NO: 38 - protein, 16 aa, synthetic construct
FYYPIWFARVLLVHYQ

SEQ ID NO: 39 - protein, 46 aa, synthetic construct
SNPFSSLFGASLLIDSVSLKSNWDTSSSSCLISFFSSVMFSSTTRS

SEQ ID NO: 40 - protein, 39 aa, synthetic construct
CRQRFSCHLTASYPQSTVTPFLAFLRRDFFFLRHNSSAD

SEQ ID NO: 41 - protein, 46 aa, synthetic construct
GAPHVVLFDFELRITNPLSHIQSVSLQITLIFCSLPSLILSKFLQV

SEQ ID NO: 42 - protein, 39 aa, synthetic construct
NTPLFSKSFSTTCGVAKKTLLLAQISSLFFLLLSSNIAV

SEQ ID NO: 43 - protein, 45 aa, synthetic construct
PTVKNSPKIFCLSSSPYLAFNLEYLSLRIFSTLSKCSNTLLTSLS

SEQ ID NO: 44 - protein, 30 aa, synthetic construct
SNQLKRLWLWLLEVRSFDRTLRRPWIHLPS

SEQ ID NO: 45 - protein, 50 aa, synthetic construct
SISFVIRSHASIRMGASNDFFHKLYFTKCLTSVILSKFLIHLLLRSTPRV

SEQ ID NO: 46 - protein, 159 aa, synthetic construct
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 47 - protein, 105 aa, synthetic construct
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLP

SEQ ID NO: 48 - protein, 26 aa, synthetic construct
RPGSTSPFAPSATDLPSMPEPALTSR

SEQ ID NO: 49 - protein, 15 aa, synthetic construct
YMSGYHTGYYMEMLA

SEQ ID NO: 50 - protein, 7 aa, synthetic construct
MSCAQES

SEQ ID NO: 51 - DNA, 24 bp, synthetic construct
  source 1..24: mol_type = other DNA
gttccggaactgcatgctcaccac

SEQ ID NO: 52 - DNA, 18 bp, synthetic construct
  source 1..18: mol_type = other DNA
cccaccaggagcagcgcc

SEQ ID NO: 53 - DNA, 19 bp, synthetic construct
  source 1..19: mol_type = other DNA
ttggaagcccgcatctatc

SEQ ID NO: 54 - DNA, 20 bp, synthetic construct
  source 1..20: mol_type = other DNA
gccacatccctaaatgagtc

SEQ ID NO: 55 - DNA, 20 bp, synthetic construct
  source 1..20: mol_type = other DNA
ctctcagcccctgtcctcag

SEQ ID NO: 56 - DNA, 25 bp, synthetic construct
  source 1..25: mol_type = other DNA
cgtggtccttatagtctacacctgt

SEQ ID NO: 57 - DNA, 20 bp, synthetic construct
  source 1..20: mol_type = other DNA
gccagccatctgttgtttgc

SEQ ID NO: 58 - DNA, 25 bp, synthetic construct
  source 1..25: mol_type = other DNA
cctgcctcagttttcctctctgtta

SEQ ID NO: 59 - DNA, 20 bp, synthetic construct
  source 1..20: mol_type = other DNA
atcatggtcatcgctttcct

SEQ ID NO: 60 - DNA, 20 bp, synthetic construct
  source 1..20: mol_type = other DNA
tcatgaagatgggaccgaag

SEQ ID NO: 61 - DNA, 20 bp, synthetic construct
  source 1..20: mol_type = other DNA
gggagaagatgacccagatc

SEQ ID NO: 62 - DNA, 3659 bp, synthetic construct
  source 1..3659: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcactagtacaccaggagacttggaacgcgggataggcacctattggtcttactgacatccactttgcctttctctccacaggtgttaataaataataaataataagccaccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggcaccagcaggaagcggagagggcagaggaagtctgctaacatgcggtgacgtcgaggagaatcctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaacaccaggagacttggaacgcggactagtaacggccgccagtgtgctggaattcaggctgacctcgagtttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaccaggagacttggaacggttttagagctagaaatagcaagttaaaataaggctagtccgttatcaacttgaaaaagtggcaccgagtcggtgcttttttgttttagagctagaaatagcaagctcgagcacctgaattctgcagatatccatcacactggcggcatccatcacactggcggcgaattcccgattaggaaagggcgaattctgcagatatccatcacactggcggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 63 - DNA, 102 bp, synthetic construct
  source 1..102: mol_type = other DNA
aagggcgatatccatcacactggcggcgaattcccgattaggaaagggcgaattctgcagatggtaccactagtaacggccgccagtgtgctggaattcagg

SEQ ID NO: 64 - DNA, 86 bp, synthetic construct
  source 1..86: mol_type = other DNA
tgtagttaatgattaacccgccatgctacttatctacgtagccatgctctaggaagatcggaattcactagtcaattggcggccgc

SEQ ID NO: 65 - DNA, 34 bp, synthetic construct
  source 1..34: mol_type = other DNA
actagtaacggccgccagtgtgctggaattcagg

SEQ ID NO: 66 - DNA, 11 bp, synthetic construct
  source 1..11: mol_type = other DNA
ctccctgccgg

SEQ ID NO: 67 - DNA, 13 bp, synthetic construct
  source 1..13: mol_type = other DNA
taagcttggatcc

SEQ ID NO: 68 - DNA, 10 bp, synthetic construct
  source 1..10: mol_type = other DNA
tgagaaccgc

SEQ ID NO: 69 - DNA, 40 bp, synthetic construct
  source 1..40: mol_type = other DNA
ggtaccactagtaacggccgccagtgtgctggaattcagg

SEQ ID NO: 70 - DNA, 249 bp, synthetic construct
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 71 - DNA, 105 bp, synthetic construct
  source 1..105: mol_type = other DNA
gttttagagctagaaatagcaagttaaaataaggctagtccgttatcaacttgaaaaagtggcaccgagtcggtgcttttttgttttagagctagaaatagcaag

SEQ ID NO: 72 - DNA, 3863 bp, synthetic construct
  source 1..3863: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcactagtcaattggcggccgcacaccaggagacttggaacgcggctccctgccggataggcacctattggtcttactgacatccactttgcctttctctccacaggtgtagactataaggaccacgacggagactacaaggatcatgatattgattacaaagacgatgacgataaggcctgcaagaactggttcagcagcctgagccacttcgtgatccacctgcgaaaaagaagaggaagcggagccaccaacttctccctgctgaagcaggccggcgacgtggaggagaaccccggccccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggcaccagcaggaagcggagagggcagaggaagtctgctaacatgcggtgacgtcgaggagaatcctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgagaaccgcggacaccaggagacttggaacgggtaccactagtaacggccgccagtgtgctggaattcagggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaccaggagacttggaacggttttagagctagaaatagcaagttaaaataaggctagtccgttatcaacttgaaaaagtggcaccgagtcggtgcttttttgttttagagctagaaatagcaagctcgagcacctgaattctgcagatatccatcacactggcggcatccatcacactggcggcgaattcccgattaggaaagggcgaattctgcagatatccatcacactggcggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 73 - DNA, 3863 bp, synthetic construct
  source 1..3863: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcactagtcaattggcggccgcacaccaggagacttggaacgcggctccctgccggataggcacctattggtcttactgacatccactttgcctttctctccacaggtgtagactataaggaccacgacggagactacaaggatcatgatattgattacaaagacgatgacgataaggcctgcaagaactggttcagcagcctgagccacttcgtgatccacctgcgaaaaagaagaggaagcggagccaccaacttctccctgctgaagcaggccggcgacgtggaggagaaccccggccccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggcaccagcaggaagcggagagggcagaggaagtctgctaacatgcggtgacgtcgaggagaatcctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgagaaccgcggacaccaggagacttggaacgggtaccactagtaacggccgccagtgtgctggaattcagggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggactcgcgcgagtcgaggaggttttagagctagaaatagcaagttaaaataaggctagtccgttatcaacttgaaaaagtggcaccgagtcggtgcttttttgttttagagctagaaatagcaagctcgagcacctgaattctgcagatatccatcacactggcggcatccatcacactggcggcgaattcccgattaggaaagggcgaattctgcagatatccatcacactggcggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2026/0137813 A1
(43) Pub. Date: May 21, 2026

(54) GENOME EDITING METHODS AND CONSTRUCTS

(71) Applicant: FONDAZIONE TELETHON ETS, Roma (IT)

(72) Inventors: Alberto Auricchio, Roma (IT); Federica Esposito, Roma (IT); Arjun Padmanabhan, Roma (IT); Ivana Trapani, Roma (IT); Manel Llado Santaeularia, Roma (IT)

(21) Appl. No.: 19/476,081

(22) PCT Filed: Apr. 22, 2024
(86) PCT No.: PCT/EP2024/060956

§ 371 Date: Oct. 16, 2025

(30) Foreign Application Priority Data
Apr. 21, 2023 (IT) 102023000007968

(51) Int. Cl. A61K 48/00; C12N 9/22; C12N 15/11; C12N 15/90

(52) U.S. Cl. CPC A61K 48/0058; C12N 9/226; C12N 15/11; C12N 15/907; C12N 2310/20; C12N 2830/50

(57) ABSTRACT

The present invention relates to a gene editing system comprising: a) a donor nucleic acid comprising: —a degradation signal sequence, —an enzymatic cleavage site, —a ribosomal skipping sequence, —an exogenous DNA sequence, wherein said donor nucleic acid is flanked at 5′ and 3′ by inverted targeting sequences; b) an oligonucleotide complementary to the targeting sequence and c) a nuclease that recognizes the targeting sequence.

### TECHNICAL FIELD

The present invention relates to genome editing methods, in particular it relates to a system comprising a donor nucleic acid comprising:
a degradation signal sequence,
an enzymatic cleavage site,
a ribosomal skipping sequence,
an exogenous DNA sequence,
   wherein said donor nucleic acid is flanked at 5′ and 3′ by inverted targeting sequences;
   and optionally an oligonucleotide complementary to the targeting sequence and/or a nuclease that recognizes the targeting sequence. The invention also refers to a method of integrating an exogenous DNA sequence into a genome of a cell comprising contacting the cell with the donor nucleic acid, an oligonucleotide complementary to the targeting sequence and a nuclease that recognizes the targeting sequence. The invention also relates to vectors comprising said donor nucleic acid and/or oligonucleotide complementary to the targeting sequence and/or nuclease and to medical uses thereof.

### BACKGROUND

Gene therapy with adeno-associated viral (AAV) vectors holds great promise to provide long-term expression of therapeutic transgenes after a single administration. However, some of the outstanding challenges include counteracting gain-of-function mutations or dominant negative effect, which do not benefit from traditional gene replacement therapy. To overcome these limitations, genome editing has emerged in the last years as a viable option for the treatment of dominantly inherited diseases, including retinal diseases (IRDs)(1). This approach relies on the usage of a nuclease discovered in the bacterial system, usually CRISPR/Cas9. Cas9 is a ribonucleoprotein that uses a short guide RNA sequence (gRNA) to recognize the target DNA by Watson-Crick base complementarity. This target DNA sequence must be adjacent to a protospacer adjacent motif (PAM) sequence for Cas9 to bind and cleave the DNA target sequence(2). This can block the production of the toxic protein without affecting the correct copy of the gene. This approach is particularly useful for the treatment of dominant form of IRDs such as autosomal dominant Retinitis Pigmentosa (adRP)(3). Retinitis Pigmentosa (RP) affects 1/3.000 patients worldwide, with 30-40% of cases having an autosomal dominant (AD) inheritance(4). The rhodopsin gene (RHO) is the most commonly mutated in AD RP patients (RP4), with the P23H mutation being the most common in US (5). RHO P23H exerts a toxic gain-of-function effect, which causes progressive degeneration of the retina and loss of vision eventually. To overcome the toxic effects of the misfolded RHO, it is necessary to disrupt the mutant P23H allele. Therefore, inventors developed a genome editing strategy to target an autosomal dominant form of Retinitis Pigmentosa due to a prevalent P23H RHO (Rhodopsin) mutation, based on the recently described homology-Independent targeted integration (HITI) strategy (6,7) and microhomology-mediated end joining (MMEJ) strategy (15). In the HITI approach, the CRISPR/Cas9 system generates the double strand breaks (DBs) into a specific site of the locus driven by a specific gRNA sequence; the resulting DBs will be mainly resolved by the non-homologous end-joining (NHEJ) repair pathway of the cell, which is the predominant repair mechanism in terminally differentiated cells such as photoreceptors and in general, is active through all the phases of the cell cycle. HITI takes advantage of the NHEJ pathway to integrate an exogenous sequence (HITI donor DNA flanked by the inverted gRNA target sites) into a specific locus at the DBs. Upon the integration of the donor DNA in the desired orientation, correct expression of the therapeutic gene will occur from the endogenous promoter.

Additionally, HITI-mediated insertion of a wild-type copy of the therapeutic gene has the potential of being therapeutic independently of the specific disease-causing mutation and could be used for treatment of dominantly inherited diseases by replacing at least the mutant allele with a correct copy of the gene provided by the donor DNA. This would avoid the target sequence restrictions imposed by allele-specificity of knockout and would broaden the applicability of the therapy to all mutations in the same gene. Inventors recently used HITI to obtain targeted knockout of RHO, independently of the mutations, followed by replacement with a healthy copy of RHO in the mouse RHO locus (7).

Microhomology (MH)-mediated end joining (MMEJ) is an alternative NHEJ (A-NHEJ) which repairs DNA double strand breaks (DBS) by annealing 2-20-bp stretches of overlapping bases flanking the DSB (15).

A previous approach for integrating an exogenous DNA sequence into a genome of a cell based on HITI is disclosed in WO2020079033, herein enclosed by reference.

However, there is still the need to improve HITI and MMEJ technologies.

### SUMMARY OF THE INVENTION

Here inventors found a surprisingly more efficient HITI and MMEJ approaches that allows the degradation of the toxic protein and the expression of the wild-type protein. The present results show the efficacy of HITI and MMEJ as therapeutic strategies for AD RP due to RHO mutations in a humanized mouse model (8) and therefore, they could be readily translated to a human setting.

Therefore, here inventors propose the HITI and MMEJ approaches as strategies to assess the therapeutic potential particularly in the human RHO locus.

New HITI construct carrying a splice acceptor sequence for efficient splicing at the target site of the RHO locus (in place of the 3×STOP codons) followed by a CL1 degradation signal (9,10) fused to an active furin cleavage site for enhanced degradation of the truncated RHO protein(11) has been evaluated. The inclusion of the CL1 degradation signal promotes a selective degradation of the 5′truncated proteins without affecting the production of full-length proteins. CL1 is further fused to P2A, a ribosomal skipping sequence, which will aid with the translation of the RHO coding sequence. Inventors then evaluated the HITI efficiency of this new construct in cells and in hRHO-P23H-TagRFP mice (8) and found that surprisingly, the levels of hRHO transcripts were approximately 2-fold higher in cells transfected with the optimized HITI donor compared to cells that were transfected with a previous HITI donor, as known from the prior art. The present gene editing system used in hRHO-P23H-TagRFP mice resulted in improved HITI efficiency up to 12±8% in the transduced area. The MMEJ construct contains the same elements of the optimized HITI donor DNA. In addition, it contains 2 different homology-arms: 1 flanking the 5′ of the splicing acceptor signal and 1 flanking the 3′ ends of the polyA sequence of the donor DNA which are homologous to the target gene. In eyes, injected with AAV-HITI gRNA and AAV-MMEJ gRNA expression cassette inventors found a significant improvement compared to AAV-scRNA injected eyes both by ER and OCT analysis.

### DETAILED DESCRIPTION OF THE INVENTION

Therefore it is an object of the invention a gene editing system comprising:
a) a donor nucleic acid comprising:
   a degradation signal sequence,
   an enzymatic cleavage site,
   a ribosomal skipping sequence,
   an exogenous DNA sequence,
wherein said donor nucleic acid is flanked at 5′ and 3′ by inverted targeting sequences;
and optionally
b) an oligonucleotide complementary to the targeting sequence (also herein defined as complementary oligonucleotide) and/or
c) a nuclease that recognizes the targeting sequence.

It also an object of the invention a gene editing system comprising:
a) a donor nucleic acid comprising:
   a degradation signal sequence,
   an enzymatic cleavage site,
   a ribosomal skipping sequence,
   an exogenous DNA sequence,
wherein said donor nucleic acid is flanked at 5′ and 3′ by inverted targeting sequences;
b) an oligonucleotide complementary to the targeting sequence and
c) a nuclease that recognizes the targeting sequence.

In the context of the present invention, the donor nucleic acid preferably further comprises a splice acceptor sequence, preferably at the 5′ of the degradation signal sequence.

Therefore the present invention also provides a gene editing system comprising:
a) a donor nucleic acid comprising:
   a splice acceptor sequence,
   a degradation signal sequence,
   an enzymatic cleavage site,
   a ribosomal skipping sequence,
   an exogenous DNA sequence,
wherein said donor nucleic acid is flanked at 5′ and 3′ by inverted targeting sequences;
and optionally
b) an oligonucleotide complementary to the targeting sequence and/or
c) a nuclease that recognizes the targeting sequence.

The gene editing system of the invention preferably comprises:
a) a donor nucleic acid comprising:
   a splice acceptor sequence,
   a degradation signal sequence,
   an enzymatic cleavage site,
   a ribosomal skipping sequence,
   an exogenous DNA sequence,
wherein said donor nucleic acid is flanked at 5′ and 3′ by inverted targeting sequences;
b) an oligonucleotide complementary to the targeting sequence and
c) a nuclease that recognizes the targeting sequence.

Preferably, the degradation signal sequence is: CL1, CL2, CL6, CL9, CL10, CL11, CL12, CL15, CL16, SL17, SMN, CIITA, ODc7, ecDHFR, PEST or a Mini ecDHFR sequence.

Preferably the degradation signal sequence is at the C-terminal position and/or it destabilizes the endogenous sequence and targets it for degradation. Preferably, the enzymatic cleavage site is selected from the group consisting of a furin cleavage site, a serine protease cleavage site, a cysteine protease cleavage site, an aspartic protease cleavage site, a metalloprotease cleavage site, and a threonine protease cleavage site, and/or it is active and/or optimized.

Preferably, the enzymatic cleavage site is a furin cleavage site, preferably active and/or optimized.

Preferably, the ribosomal skipping sequence is a ribosomal skipping sequence from Porcine Tescho virus-1 2A (P2A) or ribosomal skipping sequence from Thosea Asigna Virus 2A (T2A) or E2A or F2A sequence, preferably P2A sequence.

Preferably, splice acceptor sequence may comprise the nucleotide sequence (Y)nNYAG.

Preferably, the targeting sequence is a sequence comprised in rhodopsin (Rho) gene, more preferably said Rho gene presents one or more mutations, such as mutation(s) which causes retinitis pigmentosa 4 (RP4 (see RHO; OMIM: 180380)), or Retinitis Pigmentosa 63 (RP63 (see OMIM: 614494)).

Alternatively, the targeting sequence is a sequence comprised in a gene which is mutated in CORD1 (cone rod dystrophy 1 (see OMIM: 600624), CORD17 (cone rod dystrophy 17 (see OMIM: 615163)), BEST1(bestrophin-1;Best disease; vitelliform macular dystrophy protein 2 (see OMIM: 607854)), OPA1 (OPA1 mitochondrial dynamin like GTPase (see OMIM 605290)) or in any other gene mutated in autosomal dominant conditions.

Preferably, the targeting sequence is comprised within an intron or an exon of the gene, preferably within the first intron or exon of the gene.

Preferably, the targeting sequence is comprised within:
the first intron of RHO gene, preferably from human, mouse or pig, or
the first exon of RHO gene, preferably from human, mouse or pig.

When the targeting sequence is comprised within an exon of the gene, the splice acceptor sequence is not present.

Preferably, the exogenous DNA sequence comprises a coding sequence (preferably one or more exons or fragments thereof) of a therapeutic protein, e.g. rhodopsin, preferably it comprises one or more rhodopsin exons or fragments thereof.

Preferably, the targeting sequence is a guide RNA (gRNA) target site.

Preferably, said oligonucleotide complementary to the targeting sequence is a guide RNA that hybridizes to a targeting sequence of a gene or to its complementary strand.

Said oligonucleotide thus guides the nuclease to cut within the targeting sequence of the gene.

Preferably, said guide RNA is adjacent to a protospacer-adjacent motif (PAM) sequence.

Preferably, said oligonucleotide complementary to the targeting sequence is under the control of a promoter, preferably a U6 promoter,

Preferably, the inverted targeting sequences is an inverted sequence with respect to a target sequence and/or comprises a PAM sequence, preferably at its 3′.

Preferably, said donor nucleic acid further comprises one or more of:
a linker, preferably between the enzymatic cleavage site and the ribosomal skipping sequence;
a further ribosomal skipping sequence, preferably localized at the 3′ of the exogenous DNA sequence;
a post-transcriptional regulatory element, preferably localized at the 3′ end of the exogenous DNA sequence or of the further ribosomal skipping sequence;
a transcription termination sequence preferably localized at the 3′ end of the post-transcriptional regulatory element or at the 3′end of the exogenous DNA sequence or of the further ribosomal skipping sequence,
preferably wherein said post-transcriptional regulatory element is the Woodchuck hepatitis virus post-transcriptional regulatory element (WPRE) and/or said transcription termination sequence is a poly-adenylation signal sequence, preferably the bovine growth hormon polyA (BGH polyA) and/or said further ribosomal-skipping sequence is a T2A, P2A, E2A, F2A, preferably T2A sequence.

In an embodiment, said donor nucleic acid further comprises at least an homology arm, preferably two homology arms. More preferably, it comprises:
a first homology arm, preferably localized at the 5′ of the splice acceptor sequence,
a second homology arm, preferably localized at the 3′ of the transcription termination sequence.

Therefore, in an embodiment, said donor nucleic acid comprises, in a 5′-3′ order:
an inverted targeting sequence with its protospacer-adjacent motif (PAM) sequence;
a first homology arm
a splice acceptor sequence
a degradation signal sequence, preferably CL1 sequence,
an enzymatic cleavage site, preferably a furin cleavage site,
a ribosomal skipping sequence, preferably a P2A sequence,
an exogenous DNA sequence, preferably one or more rhodopsin exons,
a further ribosomal skipping sequence, preferably T2A,
a further exogenous DNA sequence localized at the 3′ of the further ribosomal skipping sequence;
a transcription termination sequence,
a second homology arm and
a further inverted targeting sequence with its protospacer-adjacent motif (PAM) sequence.

As mentioned above, the donor DNA sequence is flanked at 5′ and 3′ by the same gRNA target site that the gRNA recognizes, but inverted (e.g. an inverted target site or inverted targeting sequence).

In the present invention, said donor nucleic acid (or construct) preferably comprises:
an inverted targeting sequence with its protospacer-adjacent motif (PAM) sequence;
a splice acceptor sequence
a degradation signal sequence, preferably CL1 sequence,
an enzymatic cleavage site, preferably a furin cleavage site,
a ribosomal skipping sequence, preferably a P2A sequence,
an exogenous DNA sequence, preferably one or more rhodopsin exons,
a further ribosomal skipping sequence, preferably T2A,
a transcription termination sequence, and
a further inverted targeting sequence with its protospacer-adjacent motif (PAM) sequence.

Between the enzymatic cleavage site and the ribosomal skipping sequence a linker may be present.

A post-transcriptional regulatory element may be present at 5′ of the transcription termination sequence.

Preferably, said elements are in the 5′-3′ order as listed but other orders may be equally suitable.

In a preferred embodiment, said donor nucleic acid comprises in a 5′-3′ order:
a degradation signal sequence,
an enzymatic cleavage site,
a ribosomal skipping sequence,
an exogenous DNA sequence,
   wherein said donor nucleic acid is flanked at 5′ and 3′ by inverted targeting sequences.

More preferably, it comprises in a 5′-3′ order:
an inverted targeting sequence with its protospacer-adjacent motif (PAM) sequence;
a splice acceptor sequence
a degradation signal sequence, preferably CL1 sequence,
an enzymatic cleavage site, preferably a furin cleavage site,
a ribosomal skipping sequence, preferably a P2A sequence,
an exogenous DNA sequence, preferably one or more rhodopsin exons,
a further ribosomal skipping sequence, preferably T2A,
a transcription termination sequence, and
a further inverted targeting sequence with its protospacer-adjacent motif (PAM) sequence.

Preferably, the ribosomal skipping sequence comprises or has essentially a sequence having at least 80% of identity to SEQ ID NO: 1 (GCCACCAACTTCTCCCTGCTGAAGCAGGCCGGCGACGTGGAGGAGAACCCCGGCC CC) or to SEQ ID NO: 2 (GGAAGCGGAGAGGGCAGAGGAAGTCTGCTAACATGCGGTGACGTCGAGGAGAAT CCTGGACCT) or to a sequence encoding for SEQ ID NO: 3 (GSG) E G R G S L L T C G D V E E N P G P or SEQ ID NO: 4 (GSG) A T N F S L L K Q A G D V E E N P G P or functional fragments thereof and/or
the inverted targeting sequence comprises or has essentially a sequence having at least 95% of identity to SEQ ID NO: 5 (ACACCAGGAGACTTGGAACG) or functional fragments thereof and optionally comprises the SpCas9 PAM sequence (CGG) and/or
the guide RNA comprises or has essentially or is encoded by a sequence having at least 95% of identity to SEQ ID NO: 5 (ACACCAGGAGACTTGGAACG), or functional fragments thereof and/or
the oligonucleotide complementary to the targeting sequence comprises or has essentially or is encoded by a sequence having at least 95% of identity to SEQ ID NO: 5 (ACACCAGGAGACTTGGAACG) or functional fragments thereof and/or
the degradation signal sequence comprises or has essentially a sequence having at least 80% of identity to SEQ ID NO: 6 (gcctgcaagaactggttcagcagcctgagccacttcgtgatccacctg) and/or
the enzymatic cleavage site comprises or has essentially a sequence having at least 95% of identity to SEQ ID NO: 7 (CGAAAAAGAAGA) and/or
the linker comprises or has essentially a sequence having at least 95% of identity to ggaagcgga and/or
the splice acceptor sequence comprises or has essentially a sequence having at least 80% of identity to SEQ ID NO: 9 (GATAGGCACCTATTGGTCTTACTGACATCCACTTTGCCTTTCTCTCCACAGGTGT) and/or
the exogenous DNA sequence comprises or has essentially a sequence having at least 80% of identity to at least one of the following sequences: SEQ ID NO: 10

(ATGAATGGCACAGAAGGCCCTAACTTCTACGTGCCCTTCTCCAATGCGACGGGTGT
GGTACGCAGCCCCTTCGAGTACCCACAGTACTACCTGGCTGAGCCATGGCAGTTCT
CCATGCTGGCCGCCTACATGTTTCTGCTGATCGTGCTGGGCTTCCCCATCAACTTCC
TCACGCTCTACGTCACCGTCCAGCACAAGAAGCTGCGCACGCCTCTCAACTACATC
CTGCTCAACCTAGCCGTGGCTGACCTCTTCATGGTCCTAGGTGGCTTCACCAGCACC
CTCTACACCTCTCTGCATGGATACTTCGTCTTCGGGCCCACAGGATGCAATTTGGAG
GGCTTCTTTGCCACCCTGGGCG),
SEQ ID NO: 11
(GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
G);
SEQ ID NO: 12
(GTACATCCCCGAGGGCCTGCAGTGCTCGTGTGGAATCGACTACTACACGCTCAAG
CCGGAGGTCAACAACGAGTCTTTTGTCATCTACATGTTCGTGGTCCACTTCACCATC
CCCATGATTATCATCTTTTTCTGCTATGGGCAGCTCGTCTTCACCGTCAAGGAG);
SEQ ID NO: 13
(GCCGCTGCCCAGCAGCAGGAGTCAGCCACCACACAGAAGGCAGAGAAGGAGGTC
ACCCGCATGGTCATCATCATGGTCATCGCTTTCCTGATCTGCTGGGTGCCCTACGCC
AGCGTGGCATTCTACATCTTCACCCACCAGGGCTCCAACTTCGGTCCCATCTTCATG
ACCATCCCAGCGTTCTTTGCCAAGAGCGCCGCCATCTACAACCCTGTCATCTATATC
ATGATGAACAAGCAG);
SEQ ID NO: 14
(TTCCGGAACTGCATGCTCACCACCATCTGCTGCGGCAAGAACCCACTGGGTGACG
ATGAGGCCTCTGCTACCGTGTCCAAGACGGAGACGAGCCAGGTGGCACCAGCA)
and/or
the woodchuck hepatitis virus post transcriptional regulatory
element (wpre) comprises or has essentially a sequence having
at least 80% of identity to SEQ ID NO: 15
(Taagcttggatccaatcaacctctggattacaaaatttgtgaaagattgactggtattct
taactatgttgctccttttacgctatgtggatacgctgctttaatgcctttgtatcatgct
attgcttcccgtatggctttcattttctcctccttgtataaatcctggttgctgtctcttt
atgaggagttgtggcccgttgtcaggcaacgtggcgtggtgtgcactgtgtttgctgacgc
aacccccactggttggggcattgccaccacctgtcagctcctttccgggactttcgctttc
cccctccctattgccacggcggaactcatcgccgcctgccttgcccgctgctggacagggg
ctcggctgttgggcactgacaattccgtggtgttgtcggggaaatcatcgtcctttccttg
gctgctcgcctgtgttgccacctggattctgcgcgggacgtccttctgctacgtcccttcg
gccctcaatccagcggaccttccttcccgcggcctgctgccggctctgcggcctcttccgc
gtcttcg)
and/or
the bovine Growth Hormone Poly-Adenylation Signal (BGH pA)
comprises or has essentially a sequence having at least 80%
of identity to SEQ ID NO: 16
(GCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGA)
and/or
the first or second homology arm comprises or has essentially
a sequence having at least 80% or at least 90% of identity to
SEQ ID NO: 66 (ctccctgccgg) or SEQ ID NO: 68 (tgagaaccgc).

Another object of the invention is a vector that comprises the gene editing system as defined above or herein or the donor nucleic acid and/or the oligonucleotide complementary to the targeting sequence and/or a nuclease that recognizes the targeting sequence as defined above or herein.

The vector is preferably a viral vector, preferably selected from the group consisting of adeno associated vector (AAV), adenoviral vector, lentiviral vector, integrase-defective lentiviral vector, retroviral vector, or a non-viral vector, preferably selected from a polymer-based, particle-based, lipid-based, peptide-based delivery vehicle or combinations thereof, such as cationic polymers, micelles, liposomes, exosomes, microparticles and nanoparticles including lipid nanoparticles (LNP).

Preferably the vector further comprises a 5′-terminal repeat (5′-TR) nucleotide sequence and a 3′-terminal repeat (3′-TR) nucleotide sequence, preferably the 5′-TR is a 5′-inverted terminal repeat (5′-ITR) nucleotide sequence and the 3′-TR is a 3′-inverted terminal repeat (3′-ITR) nucleotide sequence.

Preferably the ITRs derive from the same virus serotype or from different virus serotypes.

Preferably the virus is an AAV, preferably of serotype 2.

A further object of the invention is a host cell comprising the gene editing system or the vector as defined herein or above.

Another object of the invention is a viral particle that comprises the gene editing system or a vector as defined above or herein.

Preferably wherein the viral particle comprises capsid proteins of an AAV. Preferably, the viral particle comprises capsid proteins of an AAV of a serotype selected from one or more of the group consisting of AAV1, AAV2, AAV3, AAV4, AAV5, AAV6, AAV7, AAV8 AAV9 and AAV 10, preferably from the AAV2 or AAV8 serotype.

A further object of the invention is a pharmaceutical composition that comprises one of the following: a gene editing system, a vector, a host cell, a viral particle as defined above or herein, and a pharmaceutically acceptable carrier.

Suitably, a viral vector as defined herein encompasses a viral vector particle.

The term “virus particle” or “viral particle” is intended to mean the extracellular form of a non-pathogenic virus, in particular a viral vector, composed of genetic material made from either DNA or RNA surrounded by a protein coat, called capsid, and in some cases an envelope derived from portions of host cell membranes and including viral glycoproteins.

As used herein, a viral vector refers also to a viral vector particle.

Viral vectors encompassed by the present invention are suitable for gene therapy.

Preferably the viral particle comprises capsid proteins of an AAV.

Preferably the viral particle comprises capsid proteins of an AAV of a serotype selected from one or more of the group consisting of AAV1, AAV2, AAV3, AAV4, AAV5, AAV6, AAV7, AAV8 AAV9, AAV 10, AAVSH19, AAVPHP.B or a derivative thereof, preferably from the AAV2 or AAV8 serotype.

Another object of the invention is a kit comprising: gene editing system, or a vector, or a host cell according, or a viral particle or a pharmaceutical composition as defined above or herein in one or more containers, optionally further comprising instructions or packaging materials that describe how to administer the nucleic acid construct, vector, host cell, viral particle or pharmaceutical composition to a patient.

Further objects of the invention are the gene editing system, or a vector, a host cell according, a viral particle or a pharmaceutical composition as defined above or herein for use as a medicament, preferably for use in in treating a genetic disease.

Further objects of the invention are the gene editing system, or a vector, a host cell, a viral particle or a pharmaceutical composition as defined above or herein for use in the treatment of autosomal dominantly inherited diseases wherein at least the mutant allele is replaced with a correct copy of the gene provided by the donor DNA or for use in treating inherited and common diseases due to toxic gain-of-function, preferably said diseases comprising retinal dystrophy, preferably the retinal dystrophy is selected from retinitis pigmentosa, cone dystrophy or cone-rod dystrophy, macular degeneration e.g. Stargardt's Disease (ELOVL4), Von-Hippel Lindau, Retinoblastoma, RP4 (see RHO; OMIM: 180380), RP63 (see OMIM: 614494), CORD1 (cone rod dystrophy 1; see OMIM: 600624), CORD17 (cone rod dystrophy 17; see OMIM: 615163), BEST1 (bestrophin-1;Best disease; vitelliform macular dystrophy protein2; see OMIM: 607854), OPA1 (OPA1 mitochondrial dynamin like GTPase; see OMIM: 605290), neuronal, hepatic diseases, metabolic disorders, lipofuscinoses (Batten's Disease and others) preferably for use in treating dominantly inherited ocular, e.g. retinal degeneration, preferably retinitis pigmentosa, neuronal and hepatic diseases. In some embodiments, both the mutant and wildtype alleles are replaced with a correct copy of the gene provided by the donor DNA.

Further objects of the invention are the gene editing system, or a vector, a host cell, a viral particle or a pharmaceutical composition as defined above or herein for use in the treatment of a genetic disease or for use in the treatment of recessive inherited diseases wherein at least one allele is replaced with a correct copy of the gene provided by the donor DNA or for use in the treatment of inherited and common diseases due to loss-of-function, preferably said diseases comprising haemophilia, diabetes, Lysosomal storage diseases comprising mucopolysaccharidoses (MPSI, MPSII, MPSIIIA, MPSIIIB, MPSIIIC, MPSIVA, MPSIVB, MPSVII), sphingolipidoses (Fabry's Disease, Gaucher Disease, Nieman-Pick Disease, GM1 Gangliosidosis), lipofuccinoses (Batten's Disease and others) and mucolipidoses; adenylosuccinate deficiency, hemophilia A and B, ALA dehydratase deficiency, adrenoleukodystrophy.

Another object of the invention is the gene editing system or of the vector or the construct as defined above or herein for the production of viral particles.

Preferably the ribosomal-skipping T2A sequence
comprises or has essentially a sequence having at
least 80% of identity to SEQ ID NO: 2
(GGAAGCGGAGAGGGCAGAGGAAGTCTGCTAACATGCGGTGACGTCGAGG
AGAATCCTGGACCT) or to a sequence encoding for SEQ
ID NO: 3 (GSG) E G R G S L L T C G D V E E N P G P
or functional fragments thereof.
Preferably the ribosomal-skipping P2A sequence
comprises or has essentially a sequence having
at least 80% of identity to SEQ ID NO: 1
(gccaccaacttctccctgctgaagcaggccggcgacgtggaggagaacc
ccggcccc) or to a sequence encoding for SEQ ID
NO: 4 (GSG) A T N F S L L K Q A G D V E E N P G P
or functional fragments thereof.

In a preferred embodiment, the oligonucleotide complementary to the targeting sequence may comprise or have essentially or be encoded by a sequence having at least 95% of identity to SEQ ID NO: 5 (ACACCAGGAGACTTGGAACG) or functional fragments thereof.

Preferably, the donor nucleic acid further comprises a polyadenylation signal, preferably a bovine growth hormone polyA.

Preferably, the targeting sequence is a sequence comprised in rhodopsin (Rho) gene. Preferably, the targeting sequence is a sequence comprised in the rhodopsin gene and the exogenous DNA sequence (or donor DNA sequence) is a coding sequence of the rhodopsin protein.

Preferably the targeting sequence is comprised within:
the first exon of RHO gene, preferably from human, mouse or pig,
the first intron of RHO gene, preferably from human, mouse or pig,
   or functional fragments thereof.

Preferably, the targeting sequence is a guide RNA (gRNA) target site and said oligonucleotide complementary to the targeting sequence is a guide RNA that hybridizes to a targeting sequence of a gene.

Said guide RNA may comprise or have essentially or be encoded by a sequence having at least 95% of identity to SEQ ID NO: 5 (ACACCAGGAGACTTGGAACG) or functional fragments thereof.

Said exogenous DNA sequence preferably comprises a reporter gene, preferably said reporter gene is selected from at least one of discosoma red (ds-RED), green fluorescent protein (GFP), a red fluorescent protein (RFP), a luciferase, a β-galactosidase and a β-glucuronidase.

Said nuclease is preferably selected from: a CRISPR nuclease, a TALEN, a DNA-guided nuclease, a meganuclease, and a Zinc Finger Nuclease, preferably said nuclease is a CRISPR nuclease selected from the group consisting of Cas9, Cpf1, Cas12b (C2c1), Cas13a (C2c2), Cas3, Csf1, Cas13b (C2c6), and C2c3 or variants thereof such as SaCas9 or VQR-Cas9-HF1.

Said complementary oligonucleotide, said donor nucleic acid, said polynucleotide encoding the nuclease are preferably comprised in a viral or non-viral vector, preferably said viral vector being selected from: an adeno-associated virus, a lentivirus, a retrovirus and an adenovirus.

Preferably the cell is selected from the group consisting of one or more of retinal cells, preferably retinal ganglion cells, bipolar cells, amacrine cells, retinal pigment epithelium, horizontal cells, rods and cones cells and preferably, cells of the anterior region of the eye such as iris pigment epithelium, corneal epithelium, corneal fibroblasts, lymphocytes, monocytes, neutrophils, eosinophils, basophils, endothelial cells, epithelial cells, hepatocytes, osteocytes, platelets, adipocytes, cardiomyocytes, neurons, smooth muscle cells, skeletal muscle cells, spermatocytes, oocytes, and pancreas cells, induced pluripotent stem cells (iPScells), stem cells, hematopoietic stem cells, hematopoietic progenitor stem cells, preferably the cell is a cell of a retina of an eye or an hepatocyte of a subject.

In a preferred embodiment the donor nucleic acid and/or the splice acceptor sequence and/or the degradation signal sequence and/or the enzymatic cleavage site and/or the ribosomal skipping sequence and/or the exogenous DNA sequence and/or the targeting sequences and/or the complementary oligonucleotide and/or the nuclease are as defined above.

Preferably, the complementary oligonucleotide and/or the donor nucleic acid and/or the polynucleotide encoding the nuclease are comprised in one or more viral or non-viral vector, preferably said viral vector being selected from: an adeno-associated virus, a retrovirus, an adenovirus and a lentivirus.

Preferably, object of the invention are the sequences herein mentioned.

For donor nucleic acid it is generally intended the nucleic acid comprising the exogenous sequence that has to be integrated in the target genome.

However, it may also be intended as comprising the oligonucleotide complementary to the targeting sequence.

In the context of the present invention, the donor DNA cassette elements and/or the gRNA expression cassette elements and/or the promoter sequences and/or U6 promoter for gRNA expression and/or the gRNA and/or the gRNA target site and/or the Cas9/Cas9-2a-GFP and/or the therapeutic transgene and/or the polyA and/or the T2A and/or P2A and/or splice acceptor sequence and/or CL1 are the sequences depicted in the following sequences 27, 30, 31, 32, 34, 62, 72 or 73 or in the sequences herein disclosed.

Preferably, a first vector comprises the donor nucleic acid and the oligonucleotide complementary to a targeting sequence and a second vector comprises the nucleic acid coding for the nuclease that recognizes said targeting sequence. Alternatively, a first vector comprises the donor nucleic acid and a second vector comprises the oligonucleotide complementary to a targeting sequence and the nucleic acid coding for the nuclease that recognizes said targeting sequence. As a further alternative, three vectors are provided: a first vector comprising the donor nucleic acid, a second vector comprising the oligonucleotide complementary to a targeting sequence and a third vector comprising the nucleic acid coding for the nuclease that recognizes said targeting sequence.

A further object of the invention is a method of integrating an exogenous DNA sequence into a genome of a cell (or into a target nucleic acid sequence in a genome), preferably of a non-diving cell, comprising contacting the cell with:
a) a donor nucleic acid comprising:
   a degradation signal sequence,
   an enzymatic cleavage site,
   a ribosomal skipping sequence,
   said exogenous DNA sequence
wherein said donor nucleic acid is flanked at 5′ and 3′ by inverted targeting sequences; and
optionally
b) an oligonucleotide complementary to the targeting sequence and/or
c) a nuclease that recognizes the targeting sequence.

Preferably, said donor nucleic acid further comprises a splice acceptor sequence, preferably at the 5′ of the degradation signal sequence.

Preferably the method of integrating an exogenous DNA sequence into a genome of a cell (or into a target nucleic acid sequence in a genome), comprises contacting the cell with:
a) a donor nucleic acid comprising:
   a splice acceptor sequence,
   a degradation signal sequence,
   an enzymatic cleavage site,
   a ribosomal skipping sequence,
   said exogenous DNA sequence
wherein said donor nucleic acid is flanked at 5′ and 3′ by inverted targeting sequences;
b) an oligonucleotide complementary to the targeting sequence and
c) a nuclease that recognizes the targeting sequence.

Preferably, the donor nucleic acid and/or the degradation signal sequence and/or the enzymatic cleavage site and/or the ribosomal skipping signal and/or the exogenous DNA sequence and/or the targeting sequences and/or the complementary oligonucleotide and/or the nuclease are as defined above or herein.

A process for preparing a viral vector particle comprising introducing such DNA constructs into a host cell, and obtaining the viral vector particle is also an object of the invention.

In a preferred embodiment the donor nucleic acid and/or the degradation signal sequence and/or the enzymatic cleavage site and/or the ribosomal skipping signal and/or the exogenous DNA sequence and/or the targeting sequences and/or the complementary strand oligonucleotide and/or the nuclease are as defined above.

Preferably, the complementary oligonucleotide and/or the donor nucleic acid and/or the polynucleotide encoding the nuclease are comprised in one or more viral or non-viral vector, preferably said viral vector being selected from: an adeno-associated virus, a retrovirus, an adenovirus and a lentivirus; said non-viral vector being preferably selected from non-viral vector is selected from a polymer-based, particle-based, lipid-based, peptide-based delivery vehicle or combinations thereof, such as cationic polymers, micelles, liposomes, exosomes, microparticles and nanoparticles including lipid nanoparticles (LNP).

Preferably, a first vector comprises the donor nucleic acid and the oligonucleotide complementary to a targeting sequence and a second vector comprises the nucleic acid coding for the nuclease that recognizes said targeting sequence. Alternatively, a first vector comprises the donor nucleic acid and a second vector comprises the oligonucleotide complementary to a targeting sequence and the nucleic acid coding for the nuclease that recognizes said targeting sequence. As a further alternative, three vectors are provided: a first vector comprising the donor nucleic acid, a second vector comprising the oligonucleotide complementary to a targeting sequence and a third vector comprising the nucleic acid coding for the nuclease that recognizes said targeting sequence.

Preferably, both the targeting sequence (defined also as target sequence) and the target nucleic acid sequence in the genome are recognized by the nuclease.

In a preferred embodiment of the invention, the target nucleic acid sequence in the genome is no longer present once the exogenous DNA sequence has been integrated into the genome of the cell (preferably a non-diving cell) in correct orientation.

In a preferred embodiment of the invention, the method does not comprise modifying the germ line genetic identity of human beings.

Preferably, said exogenous DNA sequence comprises a reporter gene, preferably said reporter gene is selected from at least one of discosoma red, green fluorescent protein (GFP), a red fluorescent protein (RFP), a luciferase, a β-galactosidase and a β-glucuronidase.

In the context of the present invention, the nuclease can be provided as a protein or as a nucleic acid coding for said nuclease. Said nucleic acid can be DNA or RNA, for example it can be the mRNA of a nuclease or it can be a cDNA or the DNA coding sequence of a nuclease or a DNA construct coding for the nuclease. Preferably, said nucleic acid coding for a nuclease is a DNA construct comprising a nucleic acid coding for Cas9 or spCas9 preferably under the control of a tissue specific promoter. Said construct may further comprise a poly A, conveniently a short synthetic polyA (sh polyA). All such elements are well known in the art and may have conventional nucleotide sequences.

Preferably, the nuclease is selected from: a CRISPR nuclease, a TALEN, a DNA-guided nuclease, a meganuclease, and a Zinc Finger Nuclease, preferably said nuclease is a CRISPR nuclease selected from the group consisting of Cas9, Cpf1, Cas12b (C2c1), Cas13a (C2c2), Cas3, Csf1, Cas13b (C2c6), and C2c3 or variants thereof such as SaCas9 or VQR-Cas9-HF1.

Suitably, said donor nucleic acid, said oligonucleotide complementary to a targeting sequence and said nucleic acid coding for said nuclease are comprised in DNA constructs. Preferably, a first DNA construct comprises the donor nucleic acid and the oligonucleotide complementary to a targeting sequence and a second DNA construct comprises the nucleic acid coding for the nuclease that recognizes said targeting sequence. Alternatively, a first DNA construct comprises the donor nucleic acid and a second DNA construct comprises the oligonucleotide complementary to a targeting sequence and the nucleic acid coding for the nuclease that recognizes said targeting sequence. As a further alternative, three constructs are provided: a first construct comprising the donor nucleic acid, a second construct comprising the oligonucleotide complementary to a targeting sequence and a third construct comprising the nucleic acid coding for the nuclease that recognizes said targeting sequence.

Said constructs are also objects of the invention.

Preferably, one or more of said DNA constructs are comprised in a vector, preferably a viral vector, still preferably a lentiviral vector or an adeno-associated vector. Alternatively, all or some of said DNA constructs may be inserted into a non-viral vector, wherein said non-viral vector is selected from a polymer-based, particle-based, lipid-based, peptide-based delivery vehicle or combinations thereof, such as cationic polymers, micelles, liposomes, exosomes, microparticles and nanoparticles including lipid nanoparticles (LNP).

Said vectors are also object of the invention.

Said complementary oligonucleotide, said donor nucleic acid and said nucleic acid encoding the nuclease can be comprised in one or more viral or non-viral vectors, preferably said viral vector being selected from: an adeno-associated virus, a lentivirus, a retrovirus and an adenovirus. This means that they can be in the same or in different vectors.

A construct comprising said donor nucleic acid and complementary oligonucleotide can comprise or have essentially a sequence having at least 80 or at least 85 or at least 90 or at least 95% of identity to SEQ ID N. 32, SEQ ID N. 30, SEQ ID N. 62 or SEQ ID N. 72.

Preferably, the complementary oligonucleotide, the donor nucleic acid and a polynucleotide encoding the nuclease are comprised in a viral or non-viral vector, preferably said viral vector being selected from: an adeno-associated virus, a lentivirus, a retrovirus and an adenovirus.

Another object of the invention is a cell obtainable by the above defined method, preferably for medical use and/or for use in treating a genetic disease, preferably for use in the treatment of autosomal dominantly inherited diseases wherein at least the mutant allele is replaced with a correct copy of the gene provided by the donor DNA, preferably both the mutant and wildtype alleles are replaced with a correct copy of the gene provided by the donor DNA, or for use in treating inherited and common diseases due to toxic gain-of-function, preferably said diseases comprising retinal dystrophy, preferably the retinal dystrophy is selected from retinitis pigmentosa, cone dystrophy or cone-rod dystrophy, macular degeneration e.g. Stargardt's Disease (ELOVL4), Von-Hippel Lindau, Retinoblastoma, RP4 (see RHO; OMIM: 180380), RP63 (see OMIM: 614494), CORD1 (cone rod dystrophy 1; see OMIM: 600624), CORD17 (cone rod dystrophy 17; see OMIM: 615163), BEST1 (bestrophin-1;Best disease; vitelliform macular dystrophy protein 2; see OMIM: 607854), OPA1 (OPA1 mitochondrial dynamin like GTPase; see OMIM: 605290), neuronal, hepatic diseases, metabolic disorders, lipofuscinoses (Batten's Disease and others) preferably for use in treating dominantly inherited ocular, e.g. retinal degeneration, preferably retinitis pigmentosa, neuronal and hepatic diseases, metabolic disorders.

Another object of the invention is a cell obtainable by the above defined method for use in the treatment of a genetic disease or for use in the treatment of recessive inherited diseases wherein at least one allele is replaced with a correct copy of the gene provided by the donor DNA or for use in the treatment of inherited and common diseases due to loss-of-function, preferably said diseases comprising haemophilia, diabetes, Lysosomal storage diseases comprising mucopolysaccharidoses (MPSI, MPSII, MPSIIIA, MPSIIIB, MPSIIIC, MPSIVA, MPSIVB, MPSVII), sphingolipidoses (Fabry's Disease, Gaucher Disease, Nieman-Pick Disease, GM1 Gangliosidosis), lipofuccinoses (Batten's Disease and others) and mucolipidoses; adenylosuccinate deficiency, hemophilia A and B, ALA dehydratase deficiency, adrenoleukodystrophy.

Preferably, the cell is selected from the group consisting of one or more of retinal cells, preferably retinal ganglion cells, bipolar cells, amacrine cells, retinal pigment epithelium, horizontal cells, rods and cones cells, or of the anterior region of the eye such as iris pigment epithelium, corneal epithelium, corneal fibroblasts, lymphocytes, monocytes, neutrophils, eosinophils, basophils, endothelial cells, epithelial cells, hepatocytes, osteocytes, platelets, adipocytes, cardiomyocytes, neurons, smooth muscle cells, skeletal muscle cells, spermatocytes, oocytes, and pancreas cells, induced pluripotent stem cells (iPScells), stem cells, hematopoietic stem cells, hematopoietic progenitor stem cells, preferably the cell is a cell of a retina of an eye or a liver cell of a subject.

A further object of the invention is a (DNA) construct comprising the donor nucleic acid and/or the oligonucleotide complementary to a targeting sequence and/or a nucleic acid coding for a nuclease that recognizes said targeting sequence, as defined herein.

In the context of the present invention the terms “oligonucleotide complementary to a targeting sequence” or “gRNA” may include DNA molecules encoding them. Therefore, the sequences herein mentioned for the oligonucleotide complementary to a targeting sequences or for the gRNA may be the sequences encoding them.

In an embodiment, the methods of the invention are ex-vivo o in vitro. In an embodiment, in the methods of the invention the cell is an isolated cell from a subject or a patient.

In an alternative embodiment, the methods of the invention are in vivo.

In an embodiment, in the methods of the invention the cell is an isolated cell from a subject or a patient.

Another object of the invention is a composition, preferably for medical use, preferably for treating the diseases/pathologies herein mentioned, comprising
a) a donor nucleic acid comprising:
   a degradation signal sequence,
   an enzymatic cleavage site,
   a ribosomal skipping sequence,
   said exogenous DNA sequence
wherein said donor nucleic acid is flanked at 5′ and 3′ by inverted targeting sequences; and
optionally,
b) an oligonucleotide complementary to the targeting sequence and/or
c) a nuclease that recognizes the targeting sequence.

Preferably, the composition comprises:
a) a donor nucleic acid comprising:
   a degradation signal sequence,
   an enzymatic cleavage site,
   a ribosomal skipping sequence,
   said exogenous DNA sequence
wherein said donor nucleic acid is flanked at 5′ and 3′ by inverted targeting sequences;
b) an oligonucleotide complementary to the targeting sequence and
c) a nuclease that recognizes the targeting sequence.

Preferably, said donor nucleic acid further comprises a splice acceptor sequence, preferably at the 5′ of the degradation signal sequence.

Therefore preferably, the composition comprises:
a) a donor nucleic acid comprising:
   a splice acceptor sequence,
   a degradation signal sequence,
   an enzymatic cleavage site,
   a ribosomal skipping sequence,
   said exogenous DNA sequence
wherein said donor nucleic acid is flanked at 5′ and 3′ by inverted targeting sequences;
b) an oligonucleotide complementary to the targeting sequence and
c) a nuclease that recognizes the targeting sequence.

Preferably, both the targeting sequence (defined also as target sequence) and the target nucleic acid sequence in the genome are recognized by the nuclease.

In a preferred embodiment of the invention, the target nucleic acid sequence in the genome is no longer present once the exogenous DNA sequence has been integrated into the genome of the cell (preferably a non-diving cell) in correct orientation.

Preferably, the elements specified in the donor nucleic acid or in the vectors or in the system are in the 5′-3′ order as listed but other orders may be equally suitable.

In a preferred embodiment of the invention, the cells are not cells of the germ line of human beings.

Preferably, the exogenous DNA sequence corrects a mutation in the genome of the cell, preferably a non-diving cell.

The inverted targeting sequences (or inverted gRNA site) in the context of the present invention are positioned upstream and downstream of the donor DNA, which is the DNA construct that is cut and then integrated in the target genome. The inverted targeting sequences are the same exact sequences as that recognized by the guide RNA in the target genomic locus i.e. the targeting sequence (e.g. rhodopsin) but inverted or reversed with respect to the genomic sequence. This allows to obtain a mono-directional integration.

Inverted or reverse may mean that if the targeting sequence has a specific 5′-3′ sequence, the inverted targeting sequence has the same sequence but with orientation 3′-5′. Therefore, an inverted targeting sequence may be complementary to the guide RNA but inverted. This allows to obtain a mono-directional integration, as known in the HITI method. In short, in the case that the donor DNA is integrated in the opposite direction the nuclease, such as Cas9, would be able to recognize again its target site and cleave it. Upon integration in the correct orientation, the nuclease would no longer be able to cleave the target site. Preferably, each of said inverted targeting sequence is linked at its 3′ to a protospacer-adjacent motif (PAM) sequence.

The genes and/or the exogenous DNA sequence and/or introns and/or exons herein disclosed may be of any origin, preferably they are from human or mouse.

In the present invention, the sequence of rhodopsin (Rho) is preferably disclosed with the following Accession numbers: human: AB065668.1, mouse: AC142099.3, pig: AEMK02000087.1.

In the present invention the exogenous DNA sequence may comprise at least one nucleotide difference compared to the genome. The exogenous DNA may not present the mutation(s) which is present in the gene which is targeted by the oligonucleotide complementary to a targeting sequence or by the gRNA.

In the present invention, guide RNA or gRNA can be used as a synonym of complementary strand oligonucleotide homologous to the targeting sequence or of complementary oligonucleotide or oligonucleotide complementary to the targeting sequence and also may refer to the DNA sequence encoding them or to the corresponding DNA molecule.

In a preferred embodiment of the invention, one vector comprising IRBP and Cas9 is used together with a second vector comprising the donor DNA as defined above

The donor DNA sequence is preferably flanked at 3′ and 5′ by the same gRNA target site that the gRNA recognizes, but inverted (e.g. an inverted target site).

The cell obtainable according to the invention expresses the exogenous sequence.

In the context of the present invention, the nuclease is preferably present in a different vector, in particular when AAV vectors are used.

Preferably AAV2/8 vectors are used.

In the present invention, a first vector comprising Cas9 or spCas9 is preferably under the control of a tissue specific promoter, e.g. a retina specific or a photoreceptor specific promoter, e.g.

Interphotoreceptor Retinoid-Binding Protein (IRBP). Said vector may further comprise a short synthetic polyA (sh polyA). Preferably, a second vector comprises the gRNA expression cassette and the donor DNA as defined herein. Preferably, the gRNA specific for rhodopsin is under the U6 promoter. Preferably the donor DNA is flanked at 3′ and 5′ by the inverted rhodopsin gRNA target sites, preferably comprising the PAM.

Preferably the above second vector may alternatively comprise the expression cassette for the rhodopsin-specific gRNA and the donor DNA comprising the coding sequence for rhodopsin, as defined above.

In a preferred embodiment, the gene of interest as well as the enzyme necessary for the NHEJ site specific insertion are carried by two AAV vectors, wherein due to the limited size of the element needed for the process, larger genes of interest may be employed. Inventors indeed minimized the structural parts (using e.g. insertions sites instead of homology arms) allowing to insert a longer cDNA in the vector.

In the context of the present invention the donor nucleic acid is inserted into the gene via nonhomologous end Joining.

The invention also provides a pharmaceutical composition comprising the nucleic acid as defined above or the nucleotide sequence as defined above or the vector as defined above and pharmaceutically acceptable diluents and/or excipients and/or carriers.

Preferably the composition further comprising a therapeutic agent, preferably the therapeutic agent is selected from the group consisting of: enzyme replacement therapy and small molecule therapy.

Preferably the pharmaceutical composition is administered through a route selected from the group consisting of: intra cerebral spinal fluid (CSF), intrathecal, parenteral, intravenous, intralesional, intraperitoneal, intramuscular, intratumoral, subcutaneous, intraventricular, intra cistema magna, lumbar, intracranial, intraspinal, intravenous, topical, nasal, oral, ocular, subretinal or any combination thereof.

The present invention also provides a vector comprising the above nucleic acid or nucleotide sequence for medical use, wherein said vector is administered through a route selected from the group consisting of: intra cerebral spinal fluid (CSF), intrathecal, parenteral, intravenous, intralesional, intraperitoneal, intramuscular, intratumoral, subcutaneous, intraventricular, intra cistema magna, lumbar, intracranial, intraspinal, intravenous, topical, nasal, oral, ocular, subretinal or any combination thereof. Preferably the vector of the invention is administered through intravenous, parenteral, ocular, preferably sub retinal route.

Preferably the vector is a viral vector, preferably the viral vector is a lentiviral vector, an integrase-defective lentiviral vector, an adeno-associated virus vector, an adenoviral vector, a retroviral vector, a polio viral vector, a murine Maloney-based viral vector, an alpha viral vector, a pox viral vector, a herpes viral vector, a vaccinia viral vector, a baculoviral vector, or a parvoviral vector, preferably the adeno-associated virus is AAV2, AAV9, AAV1, AAVSH19, AAVPHP.B, AAV8, AAV6. In the context of present invention, AAV2/8 vectors preferably are used.

Preferably the viral vector further comprises a 5′-terminal repeat (5′-TR) nucleotide sequence and a 3′-terminal repeat (3′-TR) nucleotide sequence, preferably the 5′-TR is a 5′-inverted terminal repeat (5′-ITR) nucleotide sequence and the 3′-TR is a 3′-inverted terminal repeat (3′-ITR) nucleotide sequence, preferably the ITRs derive from the same virus serotype or from different virus serotypes, preferably the virus is an AAV, preferably of serotype 2.

In an embodiment, said viral vector comprising the gRNA expression cassette and the donor DNA further comprises a 5′ inverted terminal repeat (ITR) sequence, preferably of AAV, preferably localized at the 5′ end of the construct comprising the gRNA expression cassette and the donor DNA and a 3′ inverted terminal repeat (ITR) sequence, preferably of AAV, preferably localized at the 3′ end of the construct comprising the gRNA expression cassette and the donor DNA.

Preferably, said ITR comprises or has a sequence having at least 95% of identity to SEQ ID NO 17 or 26.

Preferably said nucleotide sequence is inserted in a vector, preferably a viral vector, still preferably an adeno-associated vector.

Preferably said viral vector comprising the gRNA expression cassette and the donor DNA or the viral vector of the invention comprises:
an AAV 5′-inverted terminal repeat (5′-ITR) sequence;
an inverted targeting sequence linked to its protospacer-adjacent motif (PAM) sequence;
a splice acceptor sequence;
a degradation signal sequence,
an enzymatic cleavage site,
a ribosomal skipping sequence, preferably P2A,
the exogenous DNA sequence, preferably the coding sequence of rhodopsin gene
a ribosomal skipping sequence, preferably, T2A,
a transcription termination sequence;
a further inverted targeting sequence linked to its protospacer-adjacent motif (PAM) sequence;
an oligonucleotide complementary to the targeting sequence, under the control of a promoter, preferably the U6 promoter;
a chimeric gRNA scaffold, and
an AAV 3′-inverted terminal repeat (3′-ITR) sequence.

In an embodiment, the U6 promoter may comprise or have essentially a sequence having at least 80% of identity with SEQ ID NO: 70 (Gagggcctatttcccatgattccttcatatttgcatatacgatacaaggctgttagagagataattggaattaatttgactgtaaacacaaaga tattagtacaaaatacgtgacgtagaaagtaataatttcttgggtagtttgcagttttaaaattatgttttaaaatggactatcatatgcttaccgta acttgaaagtatttcgatttcttggctttatatatcttgtggaaaggacgaaacacc).

Suitably, when said vector does not comprise the gRNA expression cassette, said cassette is comprised in the vector comprising the nucleic acid expressing the nuclease.

The above mentioned elements may be in the order above defined, from 5′ to 3′, however other orders are equally suitable, as the skilled person can appreciate. The vector may further comprise additional viral sequences, such as additional AAV sequences.

In the present invention “at least 80% identity” means that the identity may be at least 80%, or 85% or 90% or 95% or 100% sequence identity to referred sequences. This applies to all the mentioned % of identity. In the present invention “at least 95% identity” means that the identity may be at least 95%, 96%, 97%, 98%, 99% or 100% sequence identity to referred sequences.

This applies to all the mentioned % of identity. In the present invention “at least 98% identity” means that the identity may be at least 98%, 99% or 100% sequence identity to referred sequences. This applies to all the mentioned % of identity. Preferably, the % of identity relates to the full length of the referred sequence.

Included in the present invention are also nucleic acid sequences derived from the nucleotide sequences herein mentioned, e.g. functional fragments, mutants, variants, derivatives, analogues, and sequences having a % of identity of at least 80% with the sequences herein mentioned.

The coding sequence of the present invention can codify for a variant of the gene, for example it can comprise additions, deletions or substitutions with respect to the coding sequence of the wild type gene as long as these protein variants retain substantially the same relevant functional activity as the original protein. The coding sequence can also codify for a fragment of the protein as long as this fragment retains substantially the same relevant functional activity as the original protein. Suitably, the coding sequence may be codon optimized for expression in human.

The present invention also includes embodiments wherein the sequences mentioned herein, for example the guide RNAs (or gRNA sequences) or gRNA sites or targeting sequences or inverted targeting sequence or complementary oligonucleotides, have a reverse orientation, i.e. from 3′ to 5′, or have a complementary sequence (also in a reverse orientation or a reverse complement sequence) compared to the sequences mentioned herein. Said gRNAs or gRNA sites or targeting sequences or inverted targeting sequence or oligonucleotides are also objects of the invention.

Included in the invention are also isolated guide ribonucleic acid (gRNA) comprising or consisting of a sequence that is substantially complementary or perfectly annealing to a sequence herein disclosed (in its 5′-3′ orientation or in 3′-5′ orientation) and to portions thereof at least 15 nucleotides long.

The donor nucleic acid in the present invention may comprise tag for protein detection such as 3×FLAG, preferably at 5′ of the degradation signal sequence.

Fragments in the context of the present inventions are preferably at least 15 nucleotides long.

It is also an object of the invention a method for treating a subject affected by a disease mentioned above comprising administering to the subject an effective amount of the gene editing system or the vector or the host cell or the viral particle or the pharmaceutical composition as above defined. Preferably, object of the invention are the sequences herein mentioned.

The invention will be now illustrated by means of non-limiting examples referring to the following figures.

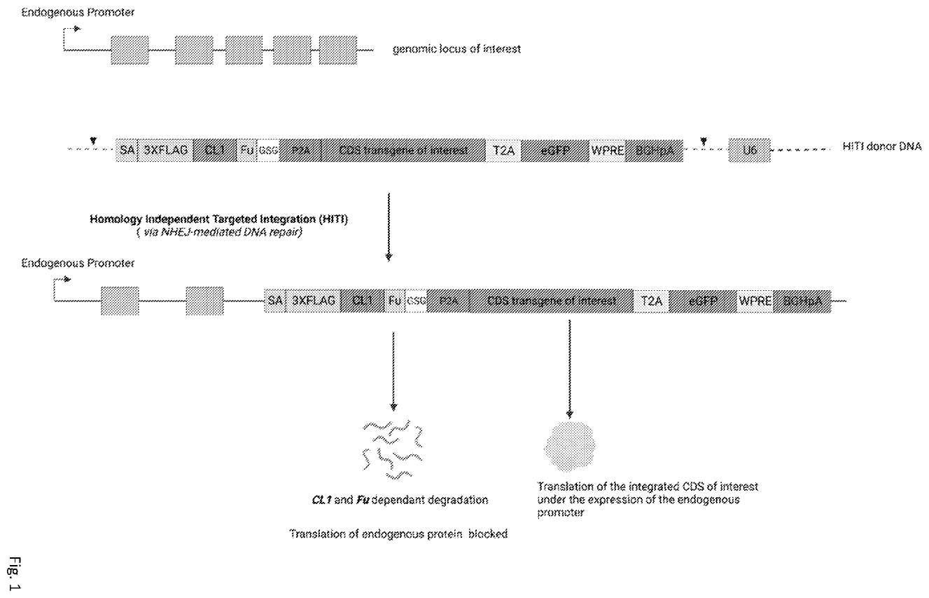

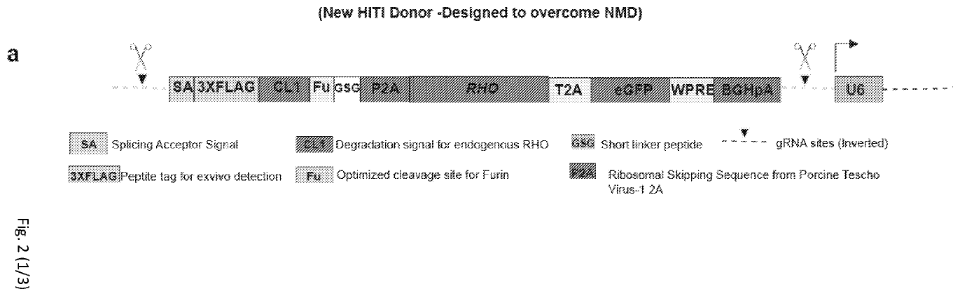

FIG. 1: Homology independent targeted integration platform. Schematic of the homology independent targeted integration strategy at a specific genomic locus of interest. Important elements of the HITI donor DNA are reported. SA=splicing acceptor sequence; 3×FLAG=tag for protein detection; CL1=degradation signal for the endogenous mutated protein; Fu=optimized cleavage site for furin; GSG=linker peptide; P2A=ribosomal skipping sequence from Porcine Tescho virus-1 2A;T2A=Ribosomal Skipping sequence from Thosea Asigna Virus 2A; eGFP=enhanced green fluorescent protein coding sequence; WPRE=woodchuck Hepatitis virus post-translational regulatory elements; BGH polya=polyadenylation signal from Bovine Growth Hormone; U6=U6 expression cassette.

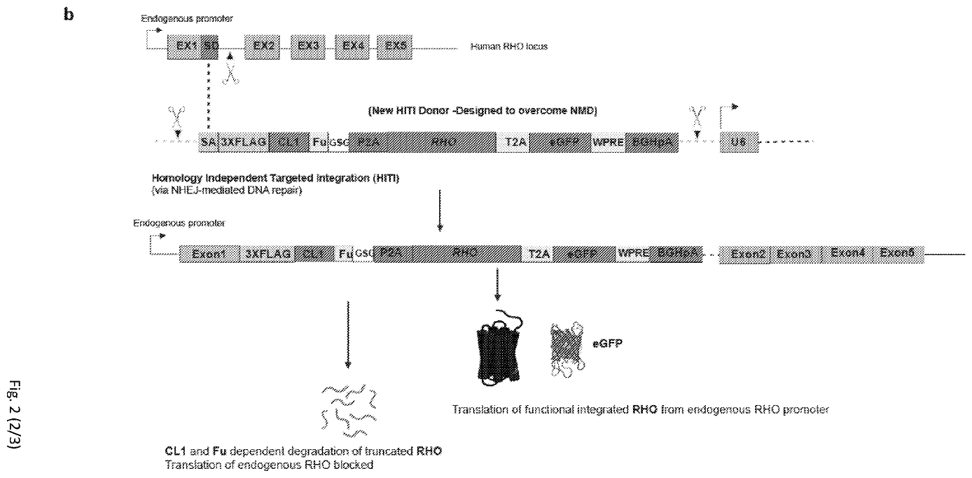

FIG. 2: Inclusion of CL1 peptide increases HITI efficiency in HEK 293 cells. (a-b) Scheme of constructs and experimental outline for evaluating the new HITI donor. Important elements of the Optm. HITI donor are reported. SA=splicing acceptor sequence; 3×FLAG=tag for protein detection; CL1=degradation signal for the endogenous mutated Rhodopsin; Fu=optimized cleavage site for furin; GSG=linker peptide; P2A=ribosomal skipping sequence from Porcine Tescho virus-1 2A; Rho=rhodopsin coding sequence; T2A=Ribosomal Skipping sequence from Thosea Asigna Virus 2A; eGFP=enhanced green fluorescent protein coding sequence; WPRE=woodchuck Hepatitis virus post-translational regulatory elements; BGH polya=polyadenylation signal from Bovine Growth Hormone; U6=human U6 promoter. (c) Relative levels of RHO transcripts in cells transfected with the Optm.HITI donor in comparison to the three tandem stop codons (3×STOP) HITI donor (IRES/Kozak). Error bars denote Standard Error of Mean. Students t-test was used to evaluate significance, p<0,05.

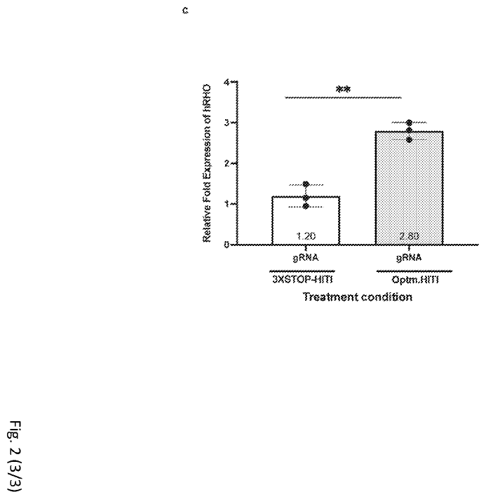

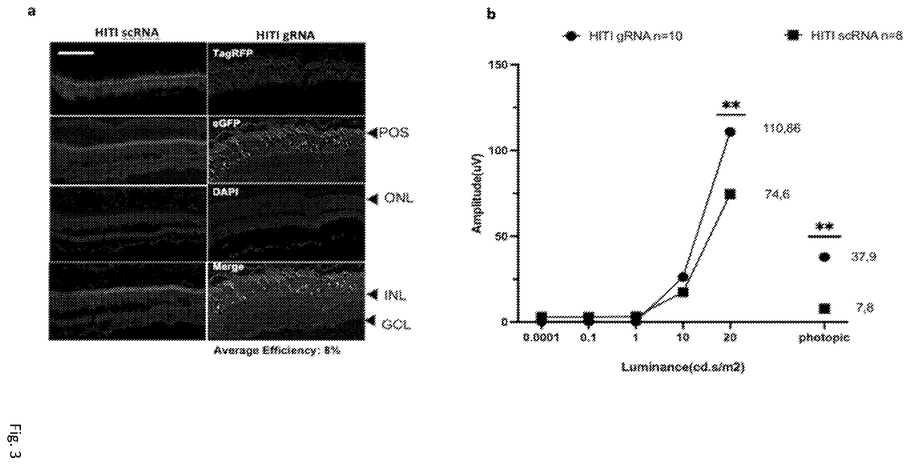

FIG. 3: Evaluation of HITI efficacy in mouse photoreceptors. A) Heterozygous mice were subretinally injected at 4 weeks of age. Representative fluorescent microscopy images of retinal OCT sections of eyes (N=4) treated with AAV-HITI gRNA; contralateral eye was treated with AAV-HITI scRNA and used as negative control. Percentage (%) of HITI efficiency is reported below. B) Electroretinogram (ERG) analysis performed 1 month post-treatment in AAV-HITI gRNA and AAV-HITI scRNA treated eyes injected at p7. Students t-test was used to evaluate significance, p<0,01.

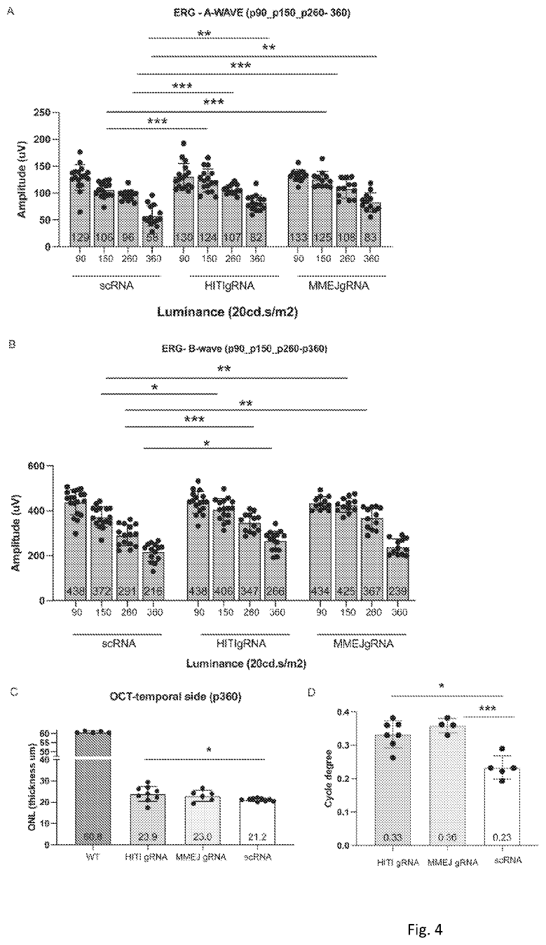

FIG. 4. therapeutic efficacy in hRHO-P23H-tagRFP heterozygous mice. A-B) Electroretinographic (ERG) analysis and OCT analysis C) were performed at different timepoints after AAV administration in eyes injected with the AAV-HITI gRNA, MMEJ gRNA or scRNA. D) Visual acuity evaluated at 1-year of age in treated mice. All data are reported as mean±SD Data are reported as mean±SD. WT=wild-type mice; heterozygous mice were injected with AAV-HITI or MMEJ donor DNA and the gRNA expression cassette (HITI gRNA and MMEJgRNA) the scRNA expression cassette was used as negative control. Statistical analysis were performed with the One-way ANOVA test.

```
Description of the Sequences
p1463_HUMAN RHO HITI Donor + U6-Scramble RNA (pAAV2.1.InvgRNA.SAS.
3XSTOP.IRES.HRHO.T2A.EGFP.PA.U6.SCR)(Size 5′ITR to 3′ITR: 3765 bp)
Components (5′ to 3′)
  1. 5′ INVERTED TERMINAL REPEATS (ITRs)
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT (SEQ ID NO: 17)
  2. Additional AAV Sequences:
TGTAGTTAATGATTAACCCGCCATGCTACTTATCTACGTAGCCATGCTCTAGGAAG
ATCGGAATTCACTAGT (SEQ ID NO: 18)
  3. Inverted gRNA sites(5′-3′): ACACCAGGAGACTTGGAACG (SEQ ID NO: 5)
  4. SpCas9 PAM 5′-3′: CGG
  5. SPLICE ACCEPTOR SEQUENCE:
GATAGGCACCTATTGGTCTTACTGACATCCACTTTGCCTTTCTCTCCACAGGTGT
(SEQ ID NO: 9)
  6. 3xSTOP CODONS: TAATAAATAATAAATAATAA (SEQ ID NO: 19)
  7. INTERNAL RIBOSOMAL ENTRY SEQUENCE(IRES):
TGACAAACTGTACATGCCGTTAACTGTAATTTTGCGTGATTTTTTTGTAG-(SEQ ID
NO: 20)
  8. Human RHODOPSIN cDNA Sequence:
  8.1 Exon1:
ATGAATGGCACAGAAGGCCCTAACTTCTACGTGCCCTTCTCCAATGCGACGGGTGT
GGTACGCAGCCCCTTCGAGTACCCACAGTACTACCTGGCTGAGCCATGGCAGTTCT
CCATGCTGGCCGCCTACATGTTTCTGCTGATCGTGCTGGGCTTCCCCATCAACTTCC
TCACGCTCTACGTCACCGTCCAGCACAAGAAGCTGCGCACGCCTCTCAACTACATC
CTGCTCAACCTAGCCGTGGCTGACCTCTTCATGGTCCTAGGTGGCTTCACCAGCACC
CTCTACACCTCTCTGCATGGATACTTCGTCTTCGGGCCCACAGGATGCAATTTGGAG
GGCTTCTTTGCCACCCTGGGCG (SEQ ID NO: 10)
  8.2 Exon 2:
GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
G (SEQ ID NO: 11)
  8.3 Exon 3:
GTACATCCCCGAGGGCCTGCAGTGCTCGTGTGGAATCGACTACTACACGCTCAAGC
CGGAGGTCAACAACGAGTCTTTTGTCATCTACATGTTCGTGGTCCACTTCACCATCC
CCATGATTATCATCTTTTTCTGCTATGGGCAGCTCGTCTTCACCGTCAAGGAG (SEQ
ID NO: 12)
  8.4 Exon 4:
GCCGCTGCCCAGCAGCAGGAGTCAGCCACCACACAGAAGGCAGAGAAGGAGGTCA
CCCGCATGGTCATCATCATGGTCATCGCTTTCCTGATCTGCTGGGTGCCCTACGCCA
GCGTGGCATTCTACATCTTCACCCACCAGGGCTCCAACTTCGGTCCCATCTTCATGA
CCATCCCAGCGTTCTTTGCCAAGAGCGCCGCCATCTACAACCCTGTCATCTATATCA
TGATGAACAAGCAG (SEQ ID NO: 13)
  8.5 Exon 5:
TTCCGGAACTGCATGCTCACCACCATCTGCTGCGGCAAGAACCCACTGGGTGACGA
TGAGGCCTCTGCTACCGTGTCCAAGACGGAGACGAGCCAGGTGGCACCAGCA (SEQ
ID NO: 14)
  9. T2A Ribosomal Skip Sequence:
GGAAGCGGAGAGGGCAGAGGAAGTCTGCTAACATGCGGTGACGTCGAGGAGAAT
CCTGGACCT (SEQ ID NO: 2)
10. enhanced Green Fluorescent Protein(eGFP):
ATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGA
ACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGG
TCCTGCTGGAGTTCGTGACCGCCGCCGGGATCACTCTCGGCATGGACGAGCTGTA
CAAGTAA (SEQ ID NO: 21)
11. woodchuck hepatitis virus post transcriptional regulatory element (wpre):
Taagcttggatccaatcaacctctggattacaaaatttgtgaaagattgactggtattcttaactatgttgctccttttacgctatgtggatacg
ctgctttaatgcctttgtatcatgctattgcttcccgtatggctttcattttctcctccttgtataaatcctggttgctgtctctttatgaggagttgtg
gcccgttgtcaggcaacgtggcgtggtgtgcactgtgtttgctgacgcaacccccactggttggggcattgccaccacctgtcagctcctt
tccgggactttcgctttccccctccctattgccacggcggaactcatcgccgcctgccttgcccgctgctggacaggggctcggctgttg
ggcactgacaattccgtggtgttgtcggggaaatcatcgtcctttccttggctgctcgcctgtgttgccacctggattctgcgcgggacgtc
cttctgctacgtcccttcggccctcaatccagcggaccttccttcccgcggcctgctgccggctctgcggcctcttccgcgtcttcg
(SEQ ID NO: 15)
12. Sequence Unknown/Stuffer sequence: AGATCT
13. Bovine Growth Hormone Poly-Adenylation Signal (BGH pA):
GCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGA (SEQ ID NO:
16)
14. Inverted gRNA sites(5′-3′): ACACCAGGAGACTTGGAACG (SEQ ID NO: 5)
15. SpCas9 PAM 5′-3′: CGG
16. Sequence Unknown/Stuffer sequence:
AAGGGCGATATCCATCACACTGGCGGCGAATTCCCGATTAGGAAAGGGCGAATTCT
GCAGATACTAGTAACGGCCGCCAGTGTGCTGGAATTCAGG (SEQ ID NO: 22)
17. U6 Expression Cassette:
ctgacctcgagtttcccatgattccttcatatttgcatatacgatacaaggctgttagagagataattggaattaatttgactgtaaacacaaa
gatattagtacaaaatacgtgacgtagaaagtaataatttcttgggtagtttgcagttttaaaattatgttttaaaatggactatcatatgcttacc
gtaacttgaaagtatttcgatttcttggctttatatatcttgtggaaaggacgaaacaccgGACTCGCGCGAGTCGAGGA
Ggttttagagctagaaatagcaagttaaaataaggctagtccgttatcaacttgaaaaagtggcaccgagtcggtgcttttttgttttagagc
tagaaatagcaag (SEQ ID NO: 23)
18. Scrambled RNA Sequence (5′-3′):
GACTCGCGCGAGTCGAGGAG
(SEQ ID NO: 24)
19. Additional AAV sequence:
CTCGAGCACCTGAATTCTGCAGATATCCATCACACTGGCGGCATCCATCACACTGG
CGGCGAATTCCCGATTAGGAAAGGGCGAATTCTGCAGATATCCATCACACTGGCGG
CGAATTCCCGATTAGGATCTTCCTAGAGCATGGCTACGTAGATAAGTAGCATGGCG
GGTTAATCATTAACTACA (SEQ ID NO: 25)
20. 3′ inverted terminal repeat sequence(3′itr)
aggaacccctagtgatggagttggccactccctctctgcgcgctcgctcgctcactgaggccgggcgaccaaaggtcgcccgacgcc
cgggctttgcccgggcggcctcagtgagcgagcgagcgcgcag
(SEQ ID NO: 26)
WHOLE SEQUENCE (5′ TO 3′) p1463_HUMAN RHO HITI Donor + U6-
ScramblegRNA
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCTTGTAGTTAATGATTAACCCGCCATGCTACTTATCTAC
GTAGCCATGCTCTAGGAAGATCGGAATTCACTAGTACACCAGGAGACTTGGAACGC
GGGATAGGCACCTATTGGTCTTACTGACATCCACTTTGCCTTTCTCTCCACAGGTGT
TAATAAATAATAAATAATAATGACAAACTGTACATGCCGTTAACTGTAATTTTGCG
TGATTTTTTTGTAGATGAATGGCACAGAAGGCCCTAACTTCTACGTGCCCTTCTCCA
ATGCGACGGGTGTGGTACGCAGCCCCTTCGAGTACCCACAGTACTACCTGGCTGAG
CCATGGCAGTTCTCCATGCTGGCCGCCTACATGTTTCTGCTGATCGTGCTGGGCTTC
CCCATCAACTTCCTCACGCTCTACGTCACCGTCCAGCACAAGAAGCTGCGCACGCC
TCTCAACTACATCCTGCTCAACCTAGCCGTGGCTGACCTCTTCATGGTCCTAGGTGG
CTTCACCAGCACCCTCTACACCTCTCTGCATGGATACTTCGTCTTCGGGCCCACAGG
ATGCAATTTGGAGGGCTTCTTTGCCACCCTGGGCGGTGAAATTGCCCTGTGGTCCTT
GGTGGTCCTGGCCATCGAGCGGTACGTGGTGGTGTGTAAGCCCATGAGCAACTTCC
GCTTCGGGGAGAACCATGCCATCATGGGCGTTGCCTTCACCTGGGTCATGGCGCTG
GCCTGCGCCGCACCCCCACTCGCCGGCTGGTCCAGGTACATCCCCGAGGGCCTGCA
GTGCTCGTGTGGAATCGACTACTACACGCTCAAGCCGGAGGTCAACAACGAGTCTT
TTGTCATCTACATGTTCGTGGTCCACTTCACCATCCCCATGATTATCATCTTTTTCTG
CTATGGGCAGCTCGTCTTCACCGTCAAGGAGGCCGCTGCCCAGCAGCAGGAGTCAG
CCACCACACAGAAGGCAGAGAAGGAGGTCACCCGCATGGTCATCATCATGGTCAT
CGCTTTCCTGATCTGCTGGGTGCCCTACGCCAGCGTGGCATTCTACATCTTCACCCA
CCAGGGCTCCAACTTCGGTCCCATCTTCATGACCATCCCAGCGTTCTTTGCCAAGAG
CGCCGCCATCTACAACCCTGTCATCTATATCATGATGAACAAGCAGTTCCGGAACT
GCATGCTCACCACCATCTGCTGCGGCAAGAACCCACTGGGTGACGATGAGGCCTCT
GCTACCGTGTCCAAGACGGAGACGAGCCAGGTGGCACCAGCAGGAAGCGGAGAGG
GCAGAGGAAGTCTGCTAACATGCGGTGACGTCGAGGAGAATCCTGGACCTATGGT
GAGCAAGGGCGAGGAGCTGTTCACCGGGGTGGTGCCCATCCTGGTCGAGCTGGAC
GGCGACGTAAACGGCCACAAGTTCAGCGTGTCCGGCGAGGGCGAGGGCGATGCCA
CCTACGGCAAGCTGACCCTGAAGTTCATCTGCACCACCGGCAAGCTGCCCGTGCCC
TGGCCCACCCTCGTGACCACCCTGACCTACGGCGTGCAGTGCTTCAGCCGCTACCC
CGACCACATGAAGCAGCACGACTTCTTCAAGTCCGCCATGCCCGAAGGCTACGTCC
AGGAGCGCACCATCTTCTTCAAGGACGACGGCAACTACAAGACCCGCGCCGAGGT
GAAGTTCGAGGGCGACACCCTGGTGAACCGCATCGAGCTGAAGGGCATCGACTTC
AAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGAT
CCGCCACAACATCGAGGACGGCAGCGTGCAGCTCGCCGACCACTACCAGCAGAAC
ACCCCCATCGGCGACGGCCCCGTGCTGCTGCCCGACAACCACTACCTGAGCACCCA
GTCCGCCCTGAGCAAAGACCCCAACGAGAAGCGCGATCACATGGTCCTGCTGGAG
TTCGTGACCGCCGCCGGGATCACTCTCGGCATGGACGAGCTGTACAAGTAAtaagcttg
gatccaatcaacctctggattacaaaatttgtgaaagattgactggtattcttaactatgttgctccttttacgctatgtggatacgctgctttaat
gcctttgtatcatgctattgcttcccgtatggctttcattttctcctccttgtataaatcctggttgctgtctctttatgaggagttgtggcccgttgtc
aggcaacgtggcgtggtgtgcactgtgtttgctgacgcaacccccactggttggggcattgccaccacctgtcagctcctttccgggacttt
cgctttccccctccctattgccacggcggaactcatcgccgcctgccttgcccgctgctggacaggggctcggctgttgggcactgacaat
tccgtggtgttgtcggggaaatcatcgtcctttccttggctgctcgcctgtgttgccacctggattctgcgcgggacgtccttctgctacgtcc
cttcggccctcaatccagcggaccttccttcccgcggcctgctgccggctctgcggcctcttccgcgtcttcgAGATCTGCCTC
GACTGTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGCATC
GCATTGTCTGAGTAGGTGTCATTCTATTCTGGGGGGTGGGGTGGGGCAGGACAGCA
AGGGGGAGGATTGGGAAGACAATAGCAGGCATGCTGGGGAACACCAGGAGACTTG
GAACGCGGAAGGGCGATATCCATCACACTGGCGGCGAATTCCCGATTAGGAAAGG
GCGAATTCTGCAGATACTAGTAACGGCCGCCAGTGTGCTGGAATTCAGGctgacctcgag
tttcccatgattccttcatatttgcatatacgatacaaggctgttagagagataattggaattaatttgactgtaaacacaaagatattagtacaa
aatacgtgacgtagaaagtaataatttcttgggtagtttgcagttttaaaattatgttttaaaatggactatcatatgcttaccgtaacttgaaagt
atttcgatttcttggctttatatatcttgtggaaaggacgaaacaccgGACTCGCGCGAGTCGAGGAGgttttagagctag
aaatagcaagttaaaataaggctagtccgttatcaacttgaaaaagtggcaccgagtcggtgcttttttgttttagagctagaaatagcaagC
TCGAGCACCTGAATTCTGCAGATATCCATCACACTGGCGGCATCCATCACACTGGC
GGCGAATTCCCGATTAGGAAAGGGCGAATTCTGCAGATATCCATCACACTGGCGGC
GAATTCCCGATTAGGATCTTCCTAGAGCATGGCTACGTAGATAAGTAGCATGGCGG
GTTAATCATTAACTACAaggaacccctagtgatggagttggccactccctctctgcgcgctcgctcgctcactgaggccg
ggcgaccaaaggtcgcccgacgcccgggctttgcccgggcggcctcagtgagcgagcgagcgcgcag
(SEQ ID NO: 27)
p1464 HUMAN RHO HITI Donor + U6-gRNA (Intron1)
pAAV2.1.InvgRNA.SAS. 3XSTOP.IRES.HRHO.T2A.EGFP.PA.U6.gRNA(Intron1)
(Size: 5′ITR to 3′ITR: 3765 bp)
Components (5′ to 3′)
  1. 5′ INVERTED TERMINAL REPEATS(ITRs)
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT (SEQ ID NO: 17)
  2. Additional AAV Sequences:
TGTAGTTAATGATTAACCCGCCATGCTACTTATCTACGTAGCCATGCTCTAGGAAG
ATCGGAATTCACTAGT (SEQ ID NO: 18)
  3. Inverted gRNA sites(5′-3′): ACACCAGGAGACTTGGAACG (SEQ ID NO: 5)
  4. SpCas9 PAM 5′: CGG
  5. SPLICE ACCEPTOR SEQUENCE:
GATAGGCACCTATTGGTCTTACTGACATCCACTTTGCCTTTCTCTCCACAGGTGT
(SEQ ID NO: 9)
  6. 3xSTOP CODONS: TAATAAATAATAAATAATAA (SEQ ID NO: 19)
  7. INTERNAL RIBOSOMAL ENTRY SEQUENCE(IRES):
TGACAAACTGTACATGCCGTTAACTGTAATTTTGCGTGATTTTTTTGTAG (SEQ ID
NO : 20)
  8. Human RHODOPSIN cDNA Sequence:
  8.1 Exon1:
ATGAATGGCACAGAAGGCCCTAACTTCTACGTGCCCTTCTCCAATGCGACGGGTGT
GGTACGCAGCCCCTTCGAGTACCCACAGTACTACCTGGCTGAGCCATGGCAGTTCT
CCATGCTGGCCGCCTACATGTTTCTGCTGATCGTGCTGGGCTTCCCCATCAACTTCC
TCACGCTCTACGTCACCGTCCAGCACAAGAAGCTGCGCACGCCTCTCAACTACATC
CTGCTCAACCTAGCCGTGGCTGACCTCTTCATGGTCCTAGGTGGCTTCACCAGCACC
CTCTACACCTCTCTGCATGGATACTTCGTCTTCGGGCCCACAGGATGCAATTTGGAG
GGCTTCTTTGCCACCCTGGGCG (SEQ ID NO: 10)
  8.2 Exon 2:
GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
G (SEQ ID NO: 11)
  8.3 Exon 3:
GTACATCCCCGAGGGCCTGCAGTGCTCGTGTGGAATCGACTACTACACGCTCAAGC
CGGAGGTCAACAACGAGTCTTTTGTCATCTACATGTTCGTGGTCCACTTCACCATCC
CCATGATTATCATCTTTTTCTGCTATGGGCAGCTCGTCTTCACCGTCAAGGAG (SEQ
ID NO: 12)
  8.4 Exon 4:
GCCGCTGCCCAGCAGCAGGAGTCAGCCACCACACAGAAGGCAGAGAAGGAGGTCA
CCCGCATGGTCATCATCATGGTCATCGCTTTCCTGATCTGCTGGGTGCCCTACGCCA
GCGTGGCATTCTACATCTTCACCCACCAGGGCTCCAACTTCGGTCCCATCTTCATGA
CCATCCCAGCGTTCTTTGCCAAGAGCGCCGCCATCTACAACCCTGTCATCTATATCA
TGATGAACAAGCAG (SEQ ID NO: 13)
  8.5 Exon 5:
TTCCGGAACTGCATGCTCACCACCATCTGCTGCGGCAAGAACCCACTGGGTGACGA
TGAGGCCTCTGCTACCGTGTCCAAGACGGAGACGAGCCAGGTGGCACCAGCA (SEQ
ID NO: 14)
  9. T2A Ribosomal Skip Sequence:
GGAAGCGGAGAGGGCAGAGGAAGTCTGCTAACATGCGGTGACGTCGAGGAGAAT
CCTGGACCT (SEQ ID NO: 2)
10. enhanced Green Fluorescent Protein(eGFP):
ATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGA
ACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGG
TCCTGCTGGAGTTCGTGACCGCCGCCGGGATCACTCTCGGCATGGACGAGCTGTA
CAAGTAA (SEQ ID NO: 21)
11. woodchuck hepatitis virus post transcriptional regulatory element (wpre):
Taagcttggatccaatcaacctctggattacaaaatttgtgaaagattgactggtattcttaactatgttgctccttttacgctatgtggatacg
ctgctttaatgcctttgtatcatgctattgcttcccgtatggctttcattttctcctccttgtataaatcctggttgctgtctctttatgaggagttgtg
gcccgttgtcaggcaacgtggcgtggtgtgcactgtgtttgctgacgcaacccccactggttggggcattgccaccacctgtcagctcctt
tccgggactttcgctttccccctccctattgccacggcggaactcatcgccgcctgccttgcccgctgctggacaggggctcggctgttg
ggcactgacaattccgtggtgttgtcggggaaatcatcgtcctttccttggctgctcgcctgtgttgccacctggattctgcgcgggacgtc
cttctgctacgtcccttcggccctcaatccagcggaccttccttcccgcggcctgctgccggctctgcggcctcttccgcgtcttcg
(SEQ ID NO: 15)
12. Sequence Unknown/Stuffer sequence: AGATCT
13. Bovine Growth Hormone Poly-Adenylation Signal (BGH pA):
GCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGA (SEQ ID NO:
16)
14. Inverted gRNA sites(5′-3′): ACACCAGGAGACTTGGAACG (SEQ ID NO: 5)
15. SpCas9 PAM 5′-3′: CGG
16. Sequence Unknown/Stuffer sequence:
AAGGGCGATATCCATCACACTGGCGGCGAATTCCCGATTAGGAAAGGGCGAATTCT
GCAGATACTAGTAACGGCCGCCAGTGTGCTGGAATTCAGG (SEQ ID NO: 22)
17. u6 expression casette:
ctgacctcgagtttcccatgattccttcatatttgcatatacgatacaaggctgttagagagataattggaattaatttgactgtaaacacaaa
gatattagtacaaaatacgtgacgtagaaagtaataatttcttgggtagtttgcagttttaaaattatgttttaaaatggactatcatatgcttacc
gtaacttgaaagtatttcgatttcttggctttatatatcttgtggaaaggacgaaacaccgACACCAGGAGACTTGGAAC
Ggttttagagctagaaatagcaagttaaaataaggctagtccgttatcaacttgaaaaagtggcaccgagtcggtgcttttttgttttagagc
tagaaatagcaag (SEQ ID NO: 28)
18. gRNA sites(5′-3′): ACACCAGGAGACTTGGAACG (SEQ ID NO: 5)
19. Additional AAV sequence:
CTCGAGCACCTGAATTCTGCAGATATCCATCACACTGGCGGCATCCATCACACTGG
CGGCGAATTCCCGATTAGGAAAGGGCGAATTCTGCAGATATCCATCACACTGGCG
GCGAATTCCCGATAAGGATCTTCCTAGAGCATGGCTACGTAGATAAGTAGCATGG
CGGGTTAATCATTAACTACA (SEQ ID NO: 29)
20. 3′ inverted terminal repeat sequence(3′itr)
Aggaacccctagtgatggagttggccactccctctctgcgcgctcgctcgctcactgaggccgggcgaccaaaggtegcccgacgcc
cgggctttgcccgggcggcctcagtgagcgagcgagcgcgcag (SEQ ID NO: 26)
WHOLE SEQUENCE (5′ TO 3′)
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT
TGTAGTTAATGATTAACCCGCCATGCTACTTATCTACGTAGCCATGCTCTAGGAAGA
TCGGAATTCACTAGTACACCAGGAGACTTGGAACGCGG
GATAGGCACCTATTGGTCTTACTGACATCCACTTTGCCTTTCTCTCCACAGGTGTTA
ATAAATAATAAATAATAATGACAAACTGTACATGCCGTTAACTGTAATTTTGCGTG
ATTTTTTTGTAG
ATGAATGGCACAGAAGGCCCTAACTTCTACGTGCCCTTCTCCAATGCGACGGGTGT
GGTACGCAGCCCCTTCGAGTACCCACAGTACTACCTGGCTGAGCCATGGCAGTTCT
CCATGCTGGCCGCCTACATGTTTCTGCTGATCGTGCTGGGCTTCCCCATCAACTTCC
TCACGCTCTACGTCACCGTCCAGCACAAGAAGCTGCGCACGCCTCTCAACTACATC
CTGCTCAACCTAGCCGTGGCTGACCTCTTCATGGTCCTAGGTGGCTTCACCAGCACC
CTCTACACCTCTCTGCATGGATACTTCGTCTTCGGGCCCACAGGATGCAATTTGGAG
GGCTTCTTTGCCACCCTGGGCGGTGAAATTGCCCTGTGGTCCTTGGTGGTCCTGGCC
ATCGAGCGGTACGTGGTGGTGTGTAAGCCCATGAGCAACTTCCGCTTCGGGGAGAA
CCATGCCATCATGGGCGTTGCCTTCACCTGGGTCATGGCGCTGGCCTGCGCCGCAC
CCCCACTCGCCGGCTGGTCCAGGTACATCCCCGAGGGCCTGCAGTGCTCGTGTGGA
ATCGACTACTACACGCTCAAGCCGGAGGTCAACAACGAGTCTTTTGTCATCTACAT
GTTCGTGGTCCACTTCACCATCCCCATGATTATCATCTTTTTCTGCTATGGGCAGCT
CGTCTTCACCGTCAAGGAGGCCGCTGCCCAGCAGCAGGAGTCAGCCACCACACAG
AAGGCAGAGAAGGAGGTCACCCGCATGGTCATCATCATGGTCATCGCTTTCCTGAT
CTGCTGGGTGCCCTACGCCAGCGTGGCATTCTACATCTTCACCCACCAGGGCTCCA
ACTTCGGTCCCATCTTCATGACCATCCCAGCGTTCTTTGCCAAGAGCGCCGCCATCT
ACAACCCTGTCATCTATATCATGATGAACAAGCAG
TTCCGGAACTGCATGCTCACCACCATCTGCTGCGGCAAGAACCCACTGGGTGACG
ATGAGGCCTCTGCTACCGTGTCCAAGACGGAGACGAGCCAGGTGGCACCAGCAGG
AAGCGGAGAGGGCAGAGGAAGTCTGCTAACATGCGGTGACGTCGAGGAGAATCC
TGGACCTATGGTGAGCAAGGGCGAGGAGCTGTTCACCGGGGTGGTGCCCATCCTG
GTCGAGCTGGACGGCGACGTAAACGGCCACAAGTTCAGCGTGTCCGGCGAGGGC
GAGGGCGATGCCACCTACGGCAAGCTGACCCTGAAGTTCATCTGCACCACCGGCA
AGCTGCCCGTGCCCTGGCCCACCCTCGTGACCACCCTGACCTACGGCGTGCAGTGC
TTCAGCCGCTACCCCGACCACATGAAGCAGCACGACTTCTTCAAGTCCGCCATGCC
CGAAGGCTACGTCCAGGAGCGCACCATCTTCTTCAAGGACGACGGCAACTACAAG
ACCCGCGCCGAGGTGAAGTTCGAGGGCGACACCCTGGTGAACCGCATCGAGCTGA
AGGGCATCGACTTCAAGGAGGACGGCAACATCCTGGGGCACAAGCTGGAGTACA
ACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAACC
ACTACCTGAGCACCCAGTCCGCCCTGAGCAAAGACCCCAACGAGAAGCGCGATCA
CATGGTCCTGCTGGAGTTCGTGACCGCCGCCGGGATCACTCTCGGCATGGACGAG
CTGTACAAGTAAtaagcttggatccaatcaacctctggattacaaaatttgtgaaagattgactggtattcttaactatgttgctcc
ttttacgctatgtggatacgctgctttaatgcctttgtatcatgctattgcttcccgtatggctttcattttctcctccttgtataaatcctggttgctg
tctctttatgaggagttgtggcccgttgtcaggcaacgtggcgtggtgtgcactgtgtttgctgacgcaacccccactggttggggcattgc
caccacctgtcagctcctttccgggactttcgctttccccctccctattgccacggcggaactcatcgccgcctgccttgcccgctgctgga
caggggctcggctgttgggcactgacaattccgtggtgttgtcggggaaatcatcgtcctttccttggctgctcgcctgtgttgccacctgg
attctgcgcgggacgtccttctgctacgtcccttcggccctcaatccagcggaccttccttcccgcggcctgctgccggctctgeggcctc
ttccgcgtcttcgAGATCT
GCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGAACACCAGG
AGACTTGGAACGCGGAAGGGCGATATCCATCACACTGGCGGCGAATTCCCGATTA
GGAAAGGGCGAATTCTGCAGATACTAGTAACGGCCGCCAGTGTGCTGGAATTCAG
Gctgacctcgagtttcccatgattccttcatatttgcatatacgatacaaggctgttagagagataattggaattaatttgactgtaaacacaa
agatattagtacaaaatacgtgacgtagaaagtaataatttcttgggtagtttgcagttttaaaattatgttttaaaatggactatcatatgcttac
cgtaacttgaaagtatttcgatttcttggctttatatatcttgtggaaaggacgaaacaccgACACCAGGAGACTTGGAAC
Ggttttagagctagaaatagcaagttaaaataaggctagtccgttatcaacttgaaaaagtggcaccgagtcggtgcttttttgttttagagc
tagaaatagcaagCTCGAGCACCTGAATTCTGCAGATATCCATCACACTGGCGGCATCCA
TCACACTGGCGGCGAATTCCCGATTAGGAAAGGGCGAATTCTGCAGATATCCATC
ACACTGGCGGCGAATTCCCGATAAGGATCTTCCTAGAGCATGGCTACGTAGATAA
GTAGCATGGGGGTTAATCATTAACTACAaggaacccctagtgatggagttggccactccctctctgcgcgc
tcgctcgctcactgaggccgggcgaccaaaggtcgcccgacgcccgggctttgcccgggcggcctcagtgagcgagcgagcgcgc
ag
(SEQ ID NO:30)
1465_HUMAN RHO HITI Donor + U6-gRNA
(Size: 5′ITR to 3′ITR: 3659 bp)
(pAAV2.1.InvgRNA.SAS.KOZAK. HRHO.T2A.EGFP.PA.U6. GRNA)
Components (5′ to 3′)
  1. 5′ INVERTED TERMINAL REPEATS(ITRs)
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT (SEQ ID NO: 17)
  2. Additional AAV Sequences:
TGTAGTTAATGATTAACCCGCCATGCTACTTATCTACGTAGCCATGCTCTAGGAAG
ATCGGAATTCACTAGT (SEQ ID NO: 18)
  3. Inverted gRNA sites(5′-3′): ACACCAGGAGACTTGGAACG (SEQ ID NO: 5)
  4. SpCas9 PAM 5′: CGG
  5. SPLICE ACCEPTOR SEQUENCE:
GATAGGCACCTATTGGTCTTACTGACATCCACTTTGCCTTTCTCTCCACAGGTGT
(SEQ ID NO: 9)
  6. 3xSTOP CODONS: TAATAAATAATAAATAATAA (SEQ ID NO: 19)
  7. KOZAK: GCCACC
  8. Human RHODOPSIN cDNA Sequence:
  8.1 Exon1:
ATGAATGGCACAGAAGGCCCTAACTTCTACGTGCCCTTCTCCAATGCGACGGGTGT
GGTACGCAGCCCCTTCGAGTACCCACAGTACTACCTGGCTGAGCCATGGCAGTTCT
CCATGCTGGCCGCCTACATGTTTCTGCTGATCGTGCTGGGCTTCCCCATCAACTTCC
TCACGCTCTACGTCACCGTCCAGCACAAGAAGCTGCGCACGCCTCTCAACTACATC
CTGCTCAACCTAGCCGTGGCTGACCTCTTCATGGTCCTAGGTGGCTTCACCAGCACC
CTCTACACCTCTCTGCATGGATACTTCGTCTTCGGGCCCACAGGATGCAATTTGGAG
GGCTTCTTTGCCACCCTGGGCG (SEQ ID NO: 10)
  8.2 Exon 2:
GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
G (SEQ ID NO: 11)
  8.3 Exon 3:
GTACATCCCCGAGGGCCTGCAGTGCTCGTGTGGAATCGACTACTACACGCTCAAGC
CGGAGGTCAACAACGAGTCTTTTGTCATCTACATGTTCGTGGTCCACTTCACCATCC
CCATGATTATCATCTTTTTCTGCTATGGGCAGCTCGTCTTCACCGTCAAGGAG (SEQ
ID NO: 12)
  8.4 Exon 4:
GCCGCTGCCCAGCAGCAGGAGTCAGCCACCACACAGAAGGCAGAGAAGGAGGTCA
CCCGCATGGTCATCATCATGGTCATCGCTTTCCTGATCTGCTGGGTGCCCTACGCCA
GCGTGGCATTCTACATCTTCACCCACCAGGGCTCCAACTTCGGTCCCATCTTCATGA
CCATCCCAGCGTTCTTTGCCAAGAGCGCCGCCATCTACAACCCTGTCATCTATATCA
TGATGAACAAGCAG (SEQ ID NO: 13)
  8.5 Exon 5:
TTCCGGAACTGCATGCTCACCACCATCTGCTGCGGCAAGAACCCACTGGGTGACGA
TGAGGCCTCTGCTACCGTGTCCAAGACGGAGACGAGCCAGGTGGCACCAGCA (SEQ
ID NO: 14)
  9. T2A Ribosomal Skip Sequence:
GGAAGCGGAGAGGGCAGAGGAAGTCTGCTAACATGCGGTGACGTCGAGGAGAAT
CCTGGACCT (SEQ ID NO: 2)
10. enhanced Green Fluorescent Protein(eGFP):
ATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGA
ACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGG
TCCTGCTGGAGTTCGTGACCGCCGCCGGGATCACTCTCGGCATGGACGAGCTGTA
CAAGTAA (SEQ ID NO: 21)
11. woodchuck hepatitis virus post transcriptional regulatory element (wpre):
Taagcttggatccaatcaacctctggattacaaaatttgtgaaagattgactggtattcttaactatgttgctccttttacgctatgtggatacgc
tgctttaatgcctttgtatcatgctattgcttcccgtatggctttcattttctcctccttgtataaatcctggttgctgtctctttatgaggagttgtggc
ccgttgtcaggcaacgtggcgtggtgtgcactgtgtttgctgacgcaacccccactggttggggcattgccaccacctgtcagctcctttcc
gggactttcgctttccccctccctattgccacggcggaactcatcgccgcctgccttgcccgctgctggacaggggctcggctgttgggca
ctgacaattccgtggtgttgtcggggaaatcatcgtcctttccttggctgctcgcctgtgttgccacctggattctgcgcgggacgtccttctg
ctacgtcccttcggccctcaatccagcggaccttccttcccgcggcctgctgccggctctgeggcctcttccgcgtcttcg (SEQ ID
NO: 15)
12. Sequence Unknown/Stuffer sequence: AGATCT
13. Bovine Growth Hormone Poly-Adenylation Signal (BGH pA):
GCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGA (SEQ ID NO:
16)
14. Inverted gRNA sites(5′-3′): ACACCAGGAGACTTGGAACG (SEQ ID NO: 5)
15. SpCas9 PAM 5′-3′: CGG
16. Sequence Unknown/Stuffer sequence:
ACTAGTAACGGCCGCCAGTGTGCTGGAATTCAGG (SEQ ID NO: 65)
17. u6 expression casette:
ctgacctcgagtttcccatgattccttcatatttgcatatacgatacaaggctgttagagagataattggaattaatttgactgtaaacacaaa
gatattagtacaaaatacgtgacgtagaaagtaataatttcttgggtagtttgcagttttaaaattatgttttaaaatggactatcatatgcttacc
gtaacttgaaagtatttcgatttcttggctttatatatcttgtggaaaggacgaaacaccgACACCAGGAGACTTGGAAC
Ggttttagagctagaaatagcaagttaaaataaggctagtccgttatcaacttgaaaaagtggcaccgagtcggtgcttttttgttttagagc
tagaaatagcaag (SEQ ID NO: 28)
18. gRNA Sequence TO INTRON1 TARGET (5′-3′):
ACACCAGGAGACTTGGAACG (SEQ ID NO: 5)
19. Additional AAV sequence:
CTCGAGCACCTGAATTCTGCAGATATCCATCACACTGGCGGCATCCATCACACTGG
CGGCGAATTCCCGATTAGGAAAGGGCGAATTCTGCAGATATCCATCACACTGGCG
GCGAATTCCCGATAAGGATCTTCCTAGAGCATGGCTACGTAGATAAGTAGCATGG
CGGGTTAATCATTAACTACA (SEQ ID NO: 29)
20. 3′ inverted terminal repeat sequence(3′itr)
Aggaacccctagtgatggagttggccactccctctctgcgcgctcgctcgctcactgaggccgggcgaccaaaggtcgcccgacgccc
gggctttgcccgggggcctcagtgagcgagcgagcgcgcag (SEQ ID NO: 26)
Complete Sequence 5-3′p1465_HUMAN RHO HITI Donor + U6-gRNA
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT
TGTAGTTAATGATTAACCCGCCATGCTACTTATCTACGTAGCCATGCTCTAGGAAG
ATCGGAATTCACTAGTACACCAGGAGACTTGGAACGCGGGATAGGCACCTATTGGT
CTTACTGACATCCACTTTGCCTTTCTCTCCACAGGTGTTAATAAATAATAAATAATA
AGCCACCATGAATGGCACAGAAGGCCCTAACTTCTACGTGCCCTTCTCCAATGCGA
CGGGTGTGGTACGCAGCCCCTTCGAGTACCCACAGTACTACCTGGCTGAGCCATGG
CAGTTCTCCATGCTGGCCGCCTACATGTTTCTGCTGATCGTGCTGGGCTTCCCCATC
AACTTCCTCACGCTCTACGTCACCGTCCAGCACAAGAAGCTGCGCACGCCTCTCAA
CTACATCCTGCTCAACCTAGCCGTGGCTGACCTCTTCATGGTCCTAGGTGGCTTCAC
CAGCACCCTCTACACCTCTCTGCATGGATACTTCGTCTTCGGGCCCACAGGATGCA
ATTTGGAGGGCTTCTTTGCCACCCTGGGCG
GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
GGTACATCCCCGAGGGCCTGCAGTGCTCGTGTGGAATCGACTACTACACGCTCAAG
CCGGAGGTCAACAACGAGTCTTTTGTCATCTACATGTTCGTGGTCCACTTCACCATC
CCCATGATTATCATCTTTTTCTGCTATGGGCAGCTCGTCTTCACCGTCAAGGAGGCC
GCTGCCCAGCAGCAGGAGTCAGCCACCACACAGAAGGCAGAGAAGGAGGTCACCC
GCATGGTCATCATCATGGTCATCGCTTTCCTGATCTGCTGGGTGCCCTACGCCAGCG
TGGCATTCTACATCTTCACCCACCAGGGCTCCAACTTCGGTCCCATCTTCATGACCA
TCCCAGCGTTCTTTGCCAAGAGCGCCGCCATCTACAACCCTGTCATCTATATCATGA
TGAACAAGCAGTTCCGGAACTGCATGCTCACCACCATCTGCTGCGGCAAGAACCCA
CTGGGTGACGATGAGGCCTCTGCTACCGTGTCCAAGACGGAGACGAGCCAGGTGG
CACCAGCAGGAAGCGGAGAGGGCAGAGGAAGTCTGCTAACATGCGGTGACGTCGA
GGAGAATCCTGGACCTATGGTGAGCAAGGGCGAGGAGCTGTTCACCGGGGTGGTG
CCCATCCTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCTGCACC
ACCGGCAAGCTGCCCGTGCCCTGGCCCACCCTCGTGACCACCCTGACCTACGGCGT
GCAGTGCTTCAGCCGCTACCCCGACCACATGAAGCAGCACGACTTCTTCAAGTCCG
CCATGCCCGAAGGCTACGTCCAGGAGCGCACCATCTTCTTCAAGGACGACGGCAAC
TACAAGACCCGCGCCGAGGTGAAGTTCGAGGGCGACACCCTGGTGAACCGCATCG
AGCTGAAGGGCATCGACTTCAAGGAGGACGGCAACATCCTGGGGCACAAGCTGGA
GTACAACTACAACAGCCACAACGTCTATATCATGGCCGACAAGCAGAAGAACGGC
ATCAAGGTGAACTTCAAGATCCGCCACAACATCGAGGACGGCAGCGTGCAGCTCG
CCGACCACTACCAGCAGAACACCCCCATCGGCGACGGCCCCGTGCTGCTGCCCGAC
AACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGAC
GAGCTGTACAAGTAAtaagcttggatccaatcaacctctggattacaaaatttgtgaaagattgactggtattcttaactatgttg
ctccttttacgctatgtggatacgctgctttaatgcctttgtatcatgctattgcttcccgtatggctttcattttctcctccttgtataaatcctggttg
ctgtctctttatgaggagttgtggcccgttgtcaggcaacgtggcgtggtgtgcactgtgtttgctgacgcaacccccactggttggggcatt
gccaccacctgtcagctcctttccgggactttcgctttccccctccctattgccacggcggaactcatcgccgcctgccttgcccgctgctg
gacaggggctcggctgttgggcactgacaattccgtggtgttgtcggggaaatcatcgtcctttccttggctgctcgcctgtgttgccacctg
gattctgcgcgggacgtccttctgctacgtcccttcggccctcaatccagcggaccttccttcccgcggcctgctgccggctctgcggcct
cttccgcgtcttcgAGATCTGCCTCGACTGTGCCTTCTAGTTGCCAGCCATCTGTTGTTTGCC
CCTCCCCCGTGCCTTCCTTGACCCTGGAAGGTGCCACTCCCACTGTCCTTTCCTAAT
AAAATGAGGAAATTGCATCGCATTGTCTGAGTAGGTGTCATTCTATTCTGGGGGGT
GGGGTGGGGCAGGACAGCAAGGGGGAGGATTGGGAAGACAATAGCAGGCATGCT
GGGGAACACCAGGAGACTTGGAACGCGG
ACTAGTAACGGCCGCCAGTGTGCTGGAATTCAGGctgacctcgagtttcccatgattccttcatatttgcata
tacgatacaaggctgttagagagataattggaattaatttgactgtaaacacaaagatattagtacaaaatacgtgacgtagaaagtaataatt
tcttgggtagtttgcagttttaaaattatgttttaaaatggactatcatatgcttaccgtaacttgaaagtatttcgatttcttggctttatatatcttgt
ggaaaggacgaaacaccgACACCAGGAGACTTGGAACGgttttagagctagaaatagcaagttaaaataaggctagt
ccgttatcaacttgaaaaagtggcaccgagtcggtgcttttttgttttagagctagaaatagcaagCTCGAGCACCTGAATTC
TGCAGATATCCATCACACTGGCGGCATCCATCACACTGGCGGCGAATTCCCGATTA
GGAAAGGGCGAATTCTGCAGATATCCATCACACTGGCGGCGAATTCCCGATAAGG
ATCTTCCTAGAGCATGGCTACGTAGATAAGTAGCATGGCGGGTTAATCATTAACTA
CAaggaacccctagtgatggagttggccactccctctctgcgcgctcgctcgctcactgaggccgggcgaccaaaggtcgcccgacg
cccgggctttgcccgggcggcctcagtgagcgagcgagcgcgcag
(SEQ ID NO: 62)
1466_HUMAN RHO HITI Donor + U6-scrambleRNA
(Size: 5′ITR to 3′ITR: 3659 bp)
(pAAV2.1.InvgRNA.SAS.KOZAK. HRHO.T2A.EGFP.PA.U6. SCRAMBLE)
Components (5′ to 3′)
  1. 5′ INVERTED TERMINAL REPEATS(ITRs)
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT (SEQ ID NO: 17)
  2. Additional AAV Sequences:
TGTAGTTAATGATTAACCCGCCATGCTACTTATCTACGTAGCCATGCTCTAGGAAG
ATCGGAATTCACTAGT (SEQ ID NO: 18)
  3. Inverted gRNA sites(5′-3′): ACACCAGGAGACTTGGAACG (SEQ ID NO: 5)
  4. SpCas9 PAM 5′: CGG
  5. SPLICE ACCEPTOR SEQUENCE:
GATAGGCACCTATTGGTCTTACTGACATCCACTTTGCCTTTCTCTCCACAGGTGT
(SEQ ID NO: 9)
  6. 3xSTOP CODONS: TAATAAATAATAAATAATAA (SEQ ID NO: 19)
  7. KOZAK: GCCACC
  8. Human RHODOPSIN cDNA Sequence:
  8.1 Exon1:
ATGAATGGCACAGAAGGCCCTAACTTCTACGTGCCCTTCTCCAATGCGACGGGTGT
GGTACGCAGCCCCTTCGAGTACCCACAGTACTACCTGGCTGAGCCATGGCAGTTCT
CCATGCTGGCCGCCTACATGTTTCTGCTGATCGTGCTGGGCTTCCCCATCAACTTCC
TCACGCTCTACGTCACCGTCCAGCACAAGAAGCTGCGCACGCCTCTCAACTACATC
CTGCTCAACCTAGCCGTGGCTGACCTCTTCATGGTCCTAGGTGGCTTCACCAGCACC
CTCTACACCTCTCTGCATGGATACTTCGTCTTCGGGCCCACAGGATGCAATTTGGAG
GGCTTCTTTGCCACCCTGGGCG (SEQ ID NO: 10)
  8.2 Exon 2:
GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
G (SEQ ID NO: 11)
  8.3 Exon 3:
GTACATCCCCGAGGGCCTGCAGTGCTCGTGTGGAATCGACTACTACACGCTCAAGC
CGGAGGTCAACAACGAGTCTTTTGTCATCTACATGTTCGTGGTCCACTTCACCATCC
CCATGATTATCATCTTTTTCTGCTATGGGCAGCTCGTCTTCACCGTCAAGGAG (SEQ
ID NO: 12)
  8.4 Exon 4:
GCCGCTGCCCAGCAGCAGGAGTCAGCCACCACACAGAAGGCAGAGAAGGAGGTCA
CCCGCATGGTCATCATCATGGTCATCGCTTTCCTGATCTGCTGGGTGCCCTACGCCA
GCGTGGCATTCTACATCTTCACCCACCAGGGCTCCAACTTCGGTCCCATCTTCATGA
CCATCCCAGCGTTCTTTGCCAAGAGCGCCGCCATCTACAACCCTGTCATCTATATCA
TGATGAACAAGCAG (SEQ ID NO: 13)
  8.5 Exon 5:
TTCCGGAACTGCATGCTCACCACCATCTGCTGCGGCAAGAACCCACTGGGTGACGA
TGAGGCCTCTGCTACCGTGTCCAAGACGGAGACGAGCCAGGTGGCACCAGCA (SEQ
ID NO: 14)
  9. T2A Ribosomal Skip Sequence:
GGAAGCGGAGAGGGCAGAGGAAGTCTGCTAACATGCGGTGACGTCGAGGAGAAT
CCTGGACCT (SEQ ID NO: 2)
10. enhanced Green Fluorescent Protein(eGFP):
ATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGA
ACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGG
TCCTGCTGGAGTTCGTGACCGCCGCCGGGATCACTCTCGGCATGGACGAGCTGTA
CAAGTAA (SEQ ID NO: 21)
11. woodchuck hepatitis virus post transcriptional regulatory element (wpre):
Taagcttggatccaatcaacctctggattacaaaatttgtgaaagattgactggtattcttaactatgttgctccttttacgctatgtggatacg
ctgctttaatgcctttgtatcatgctattgcttcccgtatggctttcattttctcctccttgtataaatcctggttgctgtctctttatgaggagttgtg
gcccgttgtcaggcaacgtggcgtggtgtgcactgtgtttgctgacgcaacccccactggttggggcattgccaccacctgtcagctcctt
tccgggactttcgctttccccctccctattgccacggcggaactcatcgccgcctgccttgcccgctgctggacaggggctcggctgttg
ggcactgacaattccgtggtgttgtcggggaaatcatcgtcctttccttggctgctcgcctgtgttgccacctggattctgcgcgggacgtc
cttctgctacgtcccttcggccctcaatccagcggaccttccttcccgcggcctgctgccggctctgeggcctcttccgcgtcttcg
(SEQ ID NO: 15)
12. Sequence Unknown/Stuffer sequence: AGATCT
13. Bovine Growth Hormone Poly-Adenylation Signal (BGH pA):
GCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAAT
TGCATCGCATTGTCTGAGTAGGTGTCATTCTATTCTGGGGGGGGGGTGGGGCAGG
ACAGCAAGGGGGAGGATTGGGAAGACAATAGCAGGCATGCTGGGGA (SEQ ID NO:
16)
14. Inverted gRNA sites(5′-3′): ACACCAGGAGACTTGGAACG (SEQ ID NO: 5)
15. SpCas9 PAM 5′-3′: CGG
16. Sequence Unknown/Stuffer sequence:
ACTAGTAACGGCCGCCAGTGTGCTGGAATTCAGG (SEQ ID NO: 65)
17. U6 Expression Casette:
ctgacctcgagtttcccatgattccttcatatttgcatatacgatacaaggctgttagagagataattggaattaatttgactgtaaacacaaa
gatattagtacaaaatacgtgacgtagaaagtaataatttcttgggtagtttgcagttttaaaattatgttttaaaatggactatcatatgcttacc
gtaacttgaaagtatttcgatttcttggctttatatatcttgtggaaaggacgaaacaccgGACTCGCGCGAGTCGAGGA
Ggttttagagctagaaatagcaagttaaaataaggctagtccgttatcaacttgaaaaagtggcaccgagtcggtgcttttttgttttagagc
tagaaatagcaag (SEQ ID NO: 23)
scramble RNA sequence (5′-3′):
GACTCGCGCGAGTCGAGGAG (SEQ ID NO: 24)
19. Additional AAV sequence:
CTCGAGCACCTGAATTCTGCAGATATCCATCACACTGGCGGCATCCATCACACTGG
CGGCGAATTCCCGATTAGGAAAGGGCGAATTCTGCAGATATCCATCACACTGGCG
GCGAATTCCCGATAAGGATCTTCCTAGAGCATGGCTACGTAGATAAGTAGCATGG
CGGGTTAATCATTAACTACA (SEQ ID NO: 29)
20. 3′ inverted terminal repeat sequence(3′itr):
Aggaacccctagtgatggagttggccactccctctctgcgcgctcgctcgctcactgaggccgggcgaccaaaggtcgcccgacgcc
cgggctttgcccgggcggcctcagtgagcgagcgagcgcgcag (SEQ ID NO: 26)
Complete Sequence (5′-3′):
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT
TGTAGTTAATGATTAACCCGCCATGCTACTTATCTACGTAGCCATGCTCTAGGAAG
ATCGGAATTCACTAGTACACCAGGAGACTTGGAACGCGGGATAGGCACCTATTGGT
CTTACTGACATCCACTTTGCCTTTCTCTCCACAGGTGTTAATAAATAATAAATAATA
AGCCACCATGAATGGCACAGAAGGCCCTAACTTCTACGTGCCCTTCTCCAATGCGA
CGGGTGTGGTACGCAGCCCCTTCGAGTACCCACAGTACTACCTGGCTGAGCCATGG
CAGTTCTCCATGCTGGCCGCCTACATGTTTCTGCTGATCGTGCTGGGCTTCCCCATC
AACTTCCTCACGCTCTACGTCACCGTCCAGCACAAGAAGCTGCGCACGCCTCTCAA
CTACATCCTGCTCAACCTAGCCGTGGCTGACCTCTTCATGGTCCTAGGTGGCTTCAC
CAGCACCCTCTACACCTCTCTGCATGGATACTTCGTCTTCGGGCCCACAGGATGCA
ATTTGGAGGGCTTCTTTGCCACCCTGGGCG
GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
GGTACATCCCCGAGGGCCTGCAGTGCTCGTGTGGAATCGACTACTACACGCTCAAG
CCGGAGGTCAACAACGAGTCTTTTGTCATCTACATGTTCGTGGTCCACTTCACCATC
CCCATGATTATCATCTTTTTCTGCTATGGGCAGCTCGTCTTCACCGTCAAGGAGGCC
GCTGCCCAGCAGCAGGAGTCAGCCACCACACAGAAGGCAGAGAAGGAGGTCACCC
GCATGGTCATCATCATGGTCATCGCTTTCCTGATCTGCTGGGTGCCCTACGCCAGCG
TGGCATTCTACATCTTCACCCACCAGGGCTCCAACTTCGGTCCCATCTTCATGACCA
TCCCAGCGTTCTTTGCCAAGAGCGCCGCCATCTACAACCCTGTCATCTATATCATGA
TGAACAAGCAGTTCCGGAACTGCATGCTCACCACCATCTGCTGCGGCAAGAACCCA
CTGGGTGACGATGAGGCCTCTGCTACCGTGTCCAAGACGGAGACGAGCCAGGTGG
CACCAGCAGGAAGCGGAGAGGGCAGAGGAAGTCTGCTAACATGCGGTGACGTCGA
GGAGAATCCTGGACCTATGGTGAGCAAGGGCGAGGAGCTGTTCACCGGGGTGGTG
CCCATCCTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCTGCACC
ACCGGCAAGCTGCCCGTGCCCTGGCCCACCCTCGTGACCACCCTGACCTACGGCGT
GCAGTGCTTCAGCCGCTACCCCGACCACATGAAGCAGCACGACTTCTTCAAGTCCG
CCATGCCCGAAGGCTACGTCCAGGAGCGCACCATCTTCTTCAAGGACGACGGCAAC
TACAAGACCCGCGCCGAGGTGAAGTTCGAGGGCGACACCCTGGTGAACCGCATCG
AGCTGAAGGGCATCGACTTCAAGGAGGACGGCAACATCCTGGGGCACAAGCTGGA
GTACAACTACAACAGCCACAACGTCTATATCATGGCCGACAAGCAGAAGAACGGC
ATCAAGGTGAACTTCAAGATCCGCCACAACATCGAGGACGGCAGCGTGCAGCTCG
CCGACCACTACCAGCAGAACACCCCCATCGGCGACGGCCCCGTGCTGCTGCCCGAC
AACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGAC
GAGCTGTACAAGTAAtaagcttggatccaatcaacctctggattacaaaatttgtgaaagattgactggtattcttaactatgttg
ctccttttacgctatgtggatacgctgctttaatgcctttgtatcatgctattgcttcccgtatggctttcattttctcctccttgtataaatcctggttg
ctgtctctttatgaggagttgtggcccgttgtcaggcaacgtggcgtggtgtgcactgtgtttgctgacgcaacccccactggttggggcatt
gccaccacctgtcagctcctttccgggactttcgctttccccctccctattgccacggcggaactcatcgccgcctgccttgcccgctgctg
gacaggggctcggctgttgggcactgacaattccgtggtgttgtcggggaaatcatcgtcctttccttggctgctcgcctgtgttgccacctg
gattctgcgcgggacgtccttctgctacgtcccttcggccctcaatccagcggaccttccttcccgcggcctgctgccggctctgcggcct
cttccgcgtcttcgAGATCTGCCTCGACTGTGCCTTCTAGTTGCCAGCCATCTGTTGTTTGCC
CCTCCCCCGTGCCTTCCTTGACCCTGGAAGGTGCCACTCCCACTGTCCTTTCCTAAT
AAAATGAGGAAATTGCATCGCATTGTCTGAGTAGGTGTCATTCTATTCTGGGGGGT
GGGGGGGGCAGGACAGCAAGGGGGAGGATTGGGAAGACAATAGCAGGCATGCT
GGGGAACACCAGGAGACTTGGAACGCGG
ACTAGTAACGGCCGCCAGTGTGCTGGAATTCAGGctgacctcgagtttcccatgattccttcatatttgcata
tacgatacaaggctgttagagagataattggaattaatttgactgtaaacacaaagatattagtacaaaatacgtgacgtagaaagtaataatt
tcttgggtagtttgcagttttaaaattatgttttaaaatggactatcatatgcttaccgtaacttgaaagtatttcgatttcttggctttatatatcttgt
ggaaaggacgaaacaccgGACTCGCGCGAGTCGAGGAGgttttagagctagaaatagcaagttaaaataaggctagt
ccgttatcaacttgaaaaagtggcaccgagtcggtgcttttttgttttagagctagaaatagcaag
CTCGAGCACCTGAATTCTGCAGATATCCATCACACTGGCGGCATCCATCACACTGG
CGGCGAATTCCCGATTAGGAAAGGGCGAATTCTGCAGATATCCATCACACTGGCG
GCGAATTCCCGATAAGGATCTTCCTAGAGCATGGCTACGTAGATAAGTAGCATGG
CGGGTTAATCATTAACTACAaggaacccctagtgatggagttggccactccctctctgcgcgctcgctcgctcactga
ggccgggcgaccaaaggtcgcccgacgcccgggctttgcccgggcggcctcagtgagcgagcgagcgcgcag
(SEQ ID NO: 31)
p1501_Optimized RHO HITI Donor + U6-gRNA
(pAAV2.1.InvgRNA.SAS.3xFLAG.CL1.Fu.GSG.P2A. HRHO.T2A.EGFP.PA.U6. GRNA)
(Size: 5′ITR to 3′ITR: 3908 bp)
Components (5′ to 3′)
  1. 5′ INVERTED TERMINAL REPEATS(ITRs)
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT (SEQ ID NO: 17)
  2. Additional AAV Sequences:
TGTAGTTAATGATTAACCCGCCATGCTACTTATCTACGTAGCCATGCTCTAGGAAG
ATCGGAATTCACTAGTCAATTGGCGGCCGC (SEQ ID NO: 64)
  3. Inverted gRNA sites(5′): ACACCAGGAGACTTGGAACG (SEQ ID NO: 5)
  4. SpCas9 PAM 5′: CGG
  5. SPLICE ACCEPTOR SEQUENCE:
GATAGGCACCTATTGGTCTTACTGACATCCACTTTGCCTTTCTCTCCACAGGTGT
(SEQ ID NO: 9)
  6. 3XFLAG:
AGACTATAAGGACCACGACGGAGACTACAAGGATCATGATATTGATTACAAAGAC
GATGACGATAAG (SEQ ID NO:33)
  7. cl1 degradation signal gcctgcaagaactggttcagcagcctgagccacttcgtgatccacctg (SEQ ID NO: 6)
OPTIMIZED FURIN CLEAVAGE SITE:
CGAAAAAGAAGA (SEQ ID NO: 7)
  8. gsg linker sequence: ggaagcgga
  9. p2a ribosomal skip sequence: gccaccaacttctccctgctgaagcaggccggcgacgtggaggagaaccccggcccc (SEQ
ID NO: 1)
10. Human RHODOPSIN cDNA Sequence:
10.1 Exon1:
ATGAATGGCACAGAAGGCCCTAACTTCTACGTGCCCTTCTCCAATGCGACGGGTGT
GGTACGCAGCCCCTTCGAGTACCCACAGTACTACCTGGCTGAGCCATGGCAGTTCT
CCATGCTGGCCGCCTACATGTTTCTGCTGATCGTGCTGGGCTTCCCCATCAACTTCC
TCACGCTCTACGTCACCGTCCAGCACAAGAAGCTGCGCACGCCTCTCAACTACATC
CTGCTCAACCTAGCCGTGGCTGACCTCTTCATGGTCCTAGGTGGCTTCACCAGCACC
CTCTACACCTCTCTGCATGGATACTTCGTCTTCGGGCCCACAGGATGCAATTTGGAG
GGCTTCTTTGCCACCCTGGGCG (SEQ ID NO: 10)
10.2 Exon 2:
GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
G (SEQ ID NO: 11)
10.3 Exon 3:
GTACATCCCCGAGGGCCTGCAGTGCTCGTGTGGAATCGACTACTACACGCTCAAGC
CGGAGGTCAACAACGAGTCTTTTGTCATCTACATGTTCGTGGTCCACTTCACCATCC
CCATGATTATCATCTTTTTCTGCTATGGGCAGCTCGTCTTCACCGTCAAGGAG (SEQ
ID NO: 12)
10.4 Exon 4:
GCCGCTGCCCAGCAGCAGGAGTCAGCCACCACACAGAAGGCAGAGAAGGAGGTCA
CCCGCATGGTCATCATCATGGTCATCGCTTTCCTGATCTGCTGGGTGCCCTACGCCA
GCGTGGCATTCTACATCTTCACCCACCAGGGCTCCAACTTCGGTCCCATCTTCATGA
CCATCCCAGCGTTCTTTGCCAAGAGCGCCGCCATCTACAACCCTGTCATCTATATCA
TGATGAACAAGCAG (SEQ ID NO: 13)
10.5 Exon 5:
TTCCGGAACTGCATGCTCACCACCATCTGCTGCGGCAAGAACCCACTGGGTGACGA
TGAGGCCTCTGCTACCGTGTCCAAGACGGAGACGAGCCAGGTGGCACCAGCA (SEQ
ID NO: 14)
11.T2A Ribosomal Skip Sequence:
GGAAGCGGAGAGGGCAGAGGAAGTCTGCTAACATGCGGTGACGTCGAGGAGAAT
CCTGGACCT (SEQ ID NO: 2)
12. enhanced Green Fluorescent Protein(eGFP):
ATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGA
ACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGG
TCCTGCTGGAGTTCGTGACCGCCGCCGGGATCACTCTCGGCATGGACGAGCTGTA
CAAGTAA (SEQ ID NO: 21)
13. woodchuck hepatitis virus post transcriptional regulatory element (wpre):
Taagcttggatccaatcaacctctggattacaaaatttgtgaaagattgactggtattcttaactatgttgctccttttacgctatgtggatacg
ctgctttaatgcctttgtatcatgctattgcttcccgtatggctttcattttctcctccttgtataaatcctggttgctgtctctttatgaggagttgtg
gcccgttgtcaggcaacgtggcgtggtgtgcactgtgtttgctgacgcaacccccactggttggggcattgccaccacctgtcagctcctt
tccgggactttcgctttccccctccctattgccacggcggaactcatcgccgcctgccttgcccgctgctggacaggggctcggctgttg
ggcactgacaattccgtggtgttgtcggggaaatcatcgtcctttccttggctgctcgcctgtgttgccacctggattctgcgcgggacgtc
cttctgctacgtcccttcggccctcaatccagcggaccttccttcccgcggcctgctgccggctctgcggcctcttccgcgtcttcg
(SEQ ID NO: 15)
14. Sequence Unknown/Stuffer sequence:
AGATCT
15. Bovine Growth Hormone Poly-Adenylation Signal (BGH pA):
GCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGA (SEQ ID NO:
16)
16. Inverted gRNA sites(5′-3′): ACACCAGGAGACTTGGAACG (SEQ ID NO: 5)
17. SpCas9 PAM 5′-3′: CGG
18. Sequence Unknown/Stuffer sequence:
AAGGGCGATATCCATCACACTGGCGGCGAATTCCCGATTAGGAAAGGGCGAATTC
TGCAGATGGTACCACTAGTAACGGCCGCCAGTGTGCTGGAATTCAGG (SEQ ID NO:
63)
19. U6 Expression Cassette
ctgacctcgagtttcccatgattccttcatatttgcatatacgatacaaggctgttagagagataattggaattaatttgactgtaaacacaaa
gatattagtacaaaatacgtgacgtagaaagtaataatttcttgggtagtttgcagttttaaaattatgttttaaaatggactatcatatgcttacc
gtaacttgaaagtatttcgatttcttggctttatatatcttgtggaaaggacgaaacaccgACACCAGGAGACTTGGAAC
Ggttttagagctagaaatagcaagttaaaataaggctagtccgttatcaacttgaaaaagtggcaccgagtcggtgcttttttgttttagagc
tagaaatagcaag (SEQ ID NO: 28)
20. gRNA Sequence TO INTRON1 TARGET (5′-3′):
ACACCAGGAGACTTGGAACG (SEQ ID NO: 5)
21. Additional AAV sequence:
CTCGAGCACCTGAATTCTGCAGATATCCATCACACTGGCGGCATCCATCACACTGG
CGGCGAATTCCCGATTAGGAAAGGGCGAATTCTGCAGATATCCATCACACTGGCG
GCGAATTCCCGATAAGGATCTTCCTAGAGCATGGCTACGTAGATAAGTAGCATGG
CGGGTTAATCATTAACTACA (SEQ ID NO: 29)
22. 3′ inverted terminal repeat sequence(3′itr):
Aggaacccctagtgatggagttggccactccctctctgcgcgctcgctcgctcactgaggccgggcgaccaaaggtegcccgacgcc
cgggctttgcccgggcggcctcagtgagcgagcgagcgcgcag (SEQ ID NO: 26)
COMPLETE SEQUENCE (5′-3′) p1501_Optimized RHO HITI Donor + U6-gRNA
(pAAV2.1.InvgRNA.SAS.3xFLAG.CL1.Fu.GSG.P2A. HRHO.T2A.EGFP.PA.U6. GRNA)
(Size: 5′ITR to 3′ITR: 3908 bp)
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT
TGTAGTTAATGATTAACCCGCCATGCTACTTATCTACGTAGCCATGCTCTAGGAAG
ATCGGAATTCACTAGTCAATTGGCGGCCGCACACCAGGAGACTTGGAACGCGGGA
TAGGCACCTATTGGTCTTACTGACATCCACTTTGCCTTTCTCTCCACAGGTGTAGA
CTATAAGGACCACGACGGAGACTACAAGGATCATGATATTGATTACAAAGACGAT
GACGATAAGgcctgcaagaactggttcagcagcctgagccacttcgtgatccacctgCGAAAAAGAAGAggaagc
ggagccaccaacttctccctgctgaagcaggccggcgacgtggaggagaaccccggccccATGAATGGCACAGAAG
GCCCTAACTTCTACGTGCCCTTCTCCAATGCGACGGGTGTGGTACGCAGCCCCTTC
GAGTACCCACAGTACTACCTGGCTGAGCCATGGCAGTTCTCCATGCTGGCCGCCTA
CATGTTTCTGCTGATCGTGCTGGGCTTCCCCATCAACTTCCTCACGCTCTACGTCAC
CGTCCAGCACAAGAAGCTGCGCACGCCTCTCAACTACATCCTGCTCAACCTAGCC
GTGGCTGACCTCTTCATGGTCCTAGGTGGCTTCACCAGCACCCTCTACACCTCTCT
GCATGGATACTTCGTCTTCGGGCCCACAGGATGCAATTTGGAGGGCTTCTTTGCCA
CCCTGGGCGGTGAAATTGCCCTGTGGTCCTTGGTGGTCCTGGCCATCGAGCGGTAC
GTGGTGGTGTGTAAGCCCATGAGCAACTTCCGCTTCGGGGAGAACCATGCCATCA
TGGGCGTTGCCTTCACCTGGGTCATGGCGCTGGCCTGCGCCGCACCCCCACTCGCC
GGCTGGTCCAGGTACATCCCCGAGGGCCTGCAGTGCTCGTGTGGAATCGACTACT
ACACGCTCAAGCCGGAGGTCAACAACGAGTCTTTTGTCATCTACATGTTCGTGGTC
CACTTCACCATCCCCATGATTATCATCTTTTTCTGCTATGGGCAGCTCGTCTTCACC
GTCAAGGAGGCCGCTGCCCAGCAGCAGGAGTCAGCCACCACACAGAAGGCAGAG
AAGGAGGTCACCCGCATGGTCATCATCATGGTCATCGCTTTCCTGATCTGCTGGGT
GCCCTACGCCAGCGTGGCATTCTACATCTTCACCCACCAGGGCTCCAACTTCGGTC
CCATCTTCATGACCATCCCAGCGTTCTTTGCCAAGAGCGCCGCCATCTACAACCCT
GTCATCTATATCATGATGAACAAGCAGTTCCGGAACTGCATGCTCACCACCATCTG
CTGCGGCAAGAACCCACTGGGTGACGATGAGGCCTCTGCTACCGTGTCCAAGACG
GAGACGAGCCAGGTGGCACCAGCAGGAAGCGGAGAGGGCAGAGGAAGTCTGCTA
ACATGCGGTGACGTCGAGGAGAATCCTGGACCTATGGTGAGCAAGGGCGAGGAG
CTGTTCACCGGGGTGGTGCCCATCCTGGTCGAGCTGGACGGCGACGTAAACGGCC
ACAAGTTCAGCGTGTCCGGCGAGGGCGAGGGCGATGCCACCTACGGCAAGCTGAC
CCTGAAGTTCATCTGCACCACCGGCAAGCTGCCCGTGCCCTGGCCCACCCTCGTGA
CCACCCTGACCTACGGCGTGCAGTGCTTCAGCCGCTACCCCGACCACATGAAGCA
GCACGACTTCTTCAAGTCCGCCATGCCCGAAGGCTACGTCCAGGAGCGCACCATC
TTCTTCAAGGACGACGGCAACTACAAGACCCGCGCCGAGGTGAAGTTCGAGGGCG
ACACCCTGGTGAACCGCATCGAGCTGAAGGGCATCGACTTCAAGGAGGACGGCA
ACATCCTGGGGCACAAGCTGGAGTACAACTACAACAGCCACAACGTCTATATCAT
GGCCGACAAGCAGAAGAACGGCATCAAGGTGAACTTCAAGATCCGCCACAACAT
CGAGGACGGCAGCGTGCAGCTCGCCGACCACTACCAGCAGAACACCCCCATCGGC
GACGGCCCCGTGCTGCTGCCCGACAACCACTACCTGAGCACCCAGTCCGCCCTGA
GCAAAGACCCCAACGAGAAGCGCGATCACATGGTCCTGCTGGAGTTCGTGACCGC
CGCCGGGATCACTCTCGGCATGGACGAGCTGTACAAGTAAtaagcttggatccaatcaacctctg
gattacaaaatttgtgaaagattgactggtattcttaactatgttgctccttttacgctatgtggatacgctgctttaatgcctttgtatcatgctat
tgcttcccgtatggctttcattttctcctccttgtataaatcctggttgctgtctctttatgaggagttgtggcccgttgtcaggcaacgtggcgt
ggtgtgcactgtgtttgctgacgcaacccccactggttggggcattgccaccacctgtcagctcctttccgggactttcgctttccccctcc
ctattgccacggcggaactcatcgccgcctgccttgcccgctgctggacaggggctcggctgttgggcactgacaattccgtggtgttgt
cggggaaatcatcgtcctttccttggctgctcgcctgtgttgccacctggattctgcgcgggacgtccttctgctacgtcccttcggccctc
aatccagcggaccttccttcccgcggcctgctgccggctctgcggcctcttccgcgtcttcgAGATCTGCCTCGACTGTG
CCTTCTAGTTGCCAGCCATCTGTTGTTTGCCCCTCCCCCGTGCCTTCCTTGACCCTG
GAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGG
GAGGATTGGGAAGACAATAGCAGGCATGCTGGGGAACACCAGGAGACTTGGAAC
GCGGAAGGGCGATATCCATCACACTGGCGGCGAATTCCCGATTAGGAAAGGGCG
AATTCTGCAGATGGTACCACTAGTAACGGCCGCCAGTGTGCTGGAATTCAGGctgacc
tcgagtttcccatgattccttcatatttgcatatacgatacaaggctgttagagagataattggaattaatttgactgtaaacacaaagatatta
gtacaaaatacgtgacgtagaaagtaataatttcttgggtagtttgcagttttaaaattatgttttaaaatggactatcatatgcttaccgtaactt
gaaagtatttcgatttcttggctttatatatcttgtggaaaggacgaaacaccgACACCAGGAGACTTGGAACGgtttta
gagctagaaatagcaagttaaaataaggctagtccgttatcaacttgaaaaagtggcaccgagtcggtgcttttttgttttagagctagaaat
agcaagCTCGAGCACCTGAATTCTGCAGATATCCATCACACTGGCGGCATCCATCAC
ACTGGCGGCGAATTCCCGATTAGGAAAGGGCGAATTCTGCAGATATCCATCACAC
TGGCGGCGAATTCCCGATAAGGATCTTCCTAGAGCATGGCTACGTAGATAAGTAG
CATGGCGGGTTAATCATTAACTACAaggaacccctagtgatggagttggccactccctctctgcgcgctcgctc
gctcactgaggccgggcgaccaaaggtcgcccgacgcccgggctttgcccgggcggcctcagtgagcgagcgagcgcgcag
(SEQ ID NO: 32)
p1503_Optimized RHO HITI Donor + U6-Scramble gRNA
(pAAV2.1.InvgRNA.SAS.3xFLAG.CL1.Fu.GSG.P2A. HRHO.T2A.EGFP.PA.U6. ScrgRNA)
(Size: 5′ITR to 3′ITR: 3908 bp)
Components (5′ to 3′)
  1. 5′ INVERTED TERMINAL REPEATS(ITRs)
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT (SEQ ID NO: 17)
  2. Additional AAV Sequences:
TGTAGTTAATGATTAACCCGCCATGCTACTTATCTACGTAGCCATGCTCTAGGAAG
ATCGGAATTCACTAGTCAATTGGCGGCCGC (SEQ ID NO: 64)
  3. Inverted gRNA sites(5′): ACACCAGGAGACTTGGAACG (SEQ ID NO: 5)
  4. SpCas9 PAM 5′: CGG
  5. SPLICE ACCEPTOR SEQUENCE:
GATAGGCACCTATTGGTCTTACTGACATCCACTTTGCCTTTCTCTCCACAGGTGT
(SEQ ID NO: 9)
  6. 3XFLAG:
AGACTATAAGGACCACGACGGAGACTACAAGGATCATGATATTGATTACAAAGAC
GATGACGATAAG (SEQ ID NO:33)
  7. cl1 degradation signal gcctgcaagaactggttcagcagcctgagccacttcgtgatccacctg (SEQ ID NO: 6)
OPTIMIZED FURIN CLEAVAGE SITE:
CGAAAAAGAAGA (SEQ ID NO: 7)
  8. gsg linker sequence: ggaagcgga
  9. p2a ribosomal skip sequence: gccaccaacttctccctgctgaagcaggccggcgacgtggaggagaaccccggcccc
(SEQ ID NO: 1)
10. Human RHODOPSIN cDNA Sequence:
10.1 Exon1:
ATGAATGGCACAGAAGGCCCTAACTTCTACGTGCCCTTCTCCAATGCGACGGGTGT
GGTACGCAGCCCCTTCGAGTACCCACAGTACTACCTGGCTGAGCCATGGCAGTTCT
CCATGCTGGCCGCCTACATGTTTCTGCTGATCGTGCTGGGCTTCCCCATCAACTTCC
TCACGCTCTACGTCACCGTCCAGCACAAGAAGCTGCGCACGCCTCTCAACTACATC
CTGCTCAACCTAGCCGTGGCTGACCTCTTCATGGTCCTAGGTGGCTTCACCAGCACC
CTCTACACCTCTCTGCATGGATACTTCGTCTTCGGGCCCACAGGATGCAATTTGGAG
GGCTTCTTTGCCACCCTGGGCG (SEQ ID NO: 10)
10.2 Exon 2:
GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
G (SEQ ID NO: 11)
10.3 Exon 3:
GTACATCCCCGAGGGCCTGCAGTGCTCGTGTGGAATCGACTACTACACGCTCAAGC
CGGAGGTCAACAACGAGTCTTTTGTCATCTACATGTTCGTGGTCCACTTCACCATCC
CCATGATTATCATCTTTTTCTGCTATGGGCAGCTCGTCTTCACCGTCAAGGAG (SEQ
ID NO: 12)
10.4 Exon 4:
GCCGCTGCCCAGCAGCAGGAGTCAGCCACCACACAGAAGGCAGAGAAGGAGGTCA
CCCGCATGGTCATCATCATGGTCATCGCTTTCCTGATCTGCTGGGTGCCCTACGCCA
GCGTGGCATTCTACATCTTCACCCACCAGGGCTCCAACTTCGGTCCCATCTTCATGA
CCATCCCAGCGTTCTTTGCCAAGAGCGCCGCCATCTACAACCCTGTCATCTATATCA
TGATGAACAAGCAG (SEQ ID NO: 13)
10.5 Exon 5:
TTCCGGAACTGCATGCTCACCACCATCTGCTGCGGCAAGAACCCACTGGGTGACGA
TGAGGCCTCTGCTACCGTGTCCAAGACGGAGACGAGCCAGGTGGCACCAGCA (SEQ
ID NO: 14)
11. T2A Ribosomal Skip Sequence:
GGAAGCGGAGAGGGCAGAGGAAGTCTGCTAACATGCGGTGACGTCGAGGAGAAT
CCTGGACCT (SEQ ID NO: 2)
12. enhanced Green Fluorescent Protein(eGFP):
ATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGA
ACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGG
TCCTGCTGGAGTTCGTGACCGCCGCCGGGATCACTCTCGGCATGGACGAGCTGTA
CAAGTAA (SEQ ID NO: 21)
13. woodchuck hepatitis virus post transcriptional regulatory element (wpre):
Taagcttggatccaatcaacctctggattacaaaatttgtgaaagattgactggtattcttaactatgttgctccttttacgctatgtggatacg
ctgctttaatgcctttgtatcatgctattgcttcccgtatggctttcattttctcctccttgtataaatcctggttgctgtctctttatgaggagttgtg
gcccgttgtcaggcaacgtggcgtggtgtgcactgtgtttgctgacgcaacccccactggttggggcattgccaccacctgtcagctcctt
tccgggactttcgctttccccctccctattgccacggcggaactcatcgccgcctgccttgcccgctgctggacaggggctcggctgttg
ggcactgacaattccgtggtgttgtcggggaaatcatcgtcctttccttggctgctcgcctgtgttgccacctggattctgcgcgggacgtc
cttctgctacgtcccttcggccctcaatccageggaccttccttcccgcggcctgctgccggctctgcggcctcttccgcgtcttcg
(SEQ ID NO: 15)
14. Sequence Unknown/Stuffer sequence:
AGATCT
15. Bovine Growth Hormone Poly-Adenylation Signal (BGH pA):
GCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGA (SEQ ID NO:
16)
16. Inverted gRNA sites(5′-3′): ACACCAGGAGACTTGGAACG (SEQ ID NO: 5)
17. SpCas9 PAM 5′-3′: CGG
18. Sequence Unknown/Stuffer sequence:
AAGGGCGATATCCATCACACTGGCGGCGAATTCCCGATTAGGAAAGGGCGAATTC
TGCAGATGGTACCACTAGTAACGGCCGCCAGTGTGCTGGAATTCAGG (SEQ ID NO:
63)
19. U6 Expression Cassette
ctgacctcgagtttcccatgattccttcatatttgcatatacgatacaaggctgttagagagataattggaattaatttgactgtaaacacaaa
gatattagtacaaaatacgtgacgtagaaagtaataatttcttgggtagtttgcagttttaaaattatgttttaaaatggactatcatatgcttacc
gtaacttgaaagtatttcgatttcttggctttatatatcttgtggaaaggacgaaacaccgGACTCGCGCGAGTCGAGGA
Ggttttagagctagaaatagcaagttaaaataaggctagtccgttatcaacttgaaaaagtggcaccgagtcggtgcttttttgttttagagc
tagaaatagcaag (SEQ ID NO: 23)
20. scramble RNA sequence (5′-3′):
GACTCGCGCGAGTCGAGGAG (SEQ ID NO: 24)
21. Additional AAV sequence:
CTCGAGCACCTGAATTCTGCAGATATCCATCACACTGGCGGCATCCATCACACTGG
CGGCGAATTCCCGATTAGGAAAGGGCGAATTCTGCAGATATCCATCACACTGGCG
GCGAATTCCCGATAAGGATCTTCCTAGAGCATGGCTACGTAGATAAGTAGCATGG
CGGGTTAATCATTAACTACA (SEQ ID NO: 29)
22. 3′ inverted terminal repeat sequence(3′itr):
Aggaacccctagtgatggagttggccactccctctctgcgcgctcgctcgctcactgaggccgggcgaccaaaggtcgcccgacgcc
cgggctttgcccgggcggcctcagtgagcgagcgagcgcgcag (SEQ ID NO: 26)
Complete Sequence (5′ to 3′) p1503_Optimized RHO HITI Donor + U6-Scramble gRNA
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCT
TGTAGTTAATGATTAACCCGCCATGCTACTTATCTACGTAGCCATGCTCTAGGAAG
ATCGGAATTCACTAGTCAATTGGCGGCCGCACACCAGGAGACTTGGAACGCGGGA
TAGGCACCTATTGGTCTTACTGACATCCACTTTGCCTTTCTCTCCACAGGTGTAGA
CTATAAGGACCACGACGGAGACTACAAGGATCATGATATTGATTACAAAGACGAT
GACGATAAGgcctgcaagaactggttcagcagcctgagccacttcgtgatccacctgCGAAAAAGAAGAggaagc
ggagccaccaacttctccctgctgaagcaggccggcgacgtggaggagaaccccggccccATGAATGGCACAGAAG
GCCCTAACTTCTACGTGCCCTTCTCCAATGCGACGGGTGTGGTACGCAGCCCCTTC
GAGTACCCACAGTACTACCTGGCTGAGCCATGGCAGTTCTCCATGCTGGCCGCCTA
CATGTTTCTGCTGATCGTGCTGGGCTTCCCCATCAACTTCCTCACGCTCTACGTCAC
CGTCCAGCACAAGAAGCTGCGCACGCCTCTCAACTACATCCTGCTCAACCTAGCC
GTGGCTGACCTCTTCATGGTCCTAGGTGGCTTCACCAGCACCCTCTACACCTCTCT
GCATGGATACTTCGTCTTCGGGCCCACAGGATGCAATTTGGAGGGCTTCTTTGCCA
CCCTGGGCGGTGAAATTGCCCTGTGGTCCTTGGTGGTCCTGGCCATCGAGCGGTAC
GTGGTGGTGTGTAAGCCCATGAGCAACTTCCGCTTCGGGGAGAACCATGCCATCA
TGGGCGTTGCCTTCACCTGGGTCATGGCGCTGGCCTGCGCCGCACCCCCACTCGCC
GGCTGGTCCAGGTACATCCCCGAGGGCCTGCAGTGCTCGTGTGGAATCGACTACT
ACACGCTCAAGCCGGAGGTCAACAACGAGTCTTTTGTCATCTACATGTTCGTGGTC
CACTTCACCATCCCCATGATTATCATCTTTTTCTGCTATGGGCAGCTCGTCTTCACC
GTCAAGGAGGCCGCTGCCCAGCAGCAGGAGTCAGCCACCACACAGAAGGCAGAG
AAGGAGGTCACCCGCATGGTCATCATCATGGTCATCGCTTTCCTGATCTGCTGGGT
GCCCTACGCCAGCGTGGCATTCTACATCTTCACCCACCAGGGCTCCAACTTCGGTC
CCATCTTCATGACCATCCCAGCGTTCTTTGCCAAGAGCGCCGCCATCTACAACCCT
GTCATCTATATCATGATGAACAAGCAGTTCCGGAACTGCATGCTCACCACCATCTG
CTGCGGCAAGAACCCACTGGGTGACGATGAGGCCTCTGCTACCGTGTCCAAGACG
GAGACGAGCCAGGTGGCACCAGCAGGAAGCGGAGAGGGCAGAGGAAGTCTGCTA
ACATGCGGTGACGTCGAGGAGAATCCTGGACCTATGGTGAGCAAGGGCGAGGAG
CTGTTCACCGGGGTGGTGCCCATCCTGGTCGAGCTGGACGGCGACGTAAACGGCC
ACAAGTTCAGCGTGTCCGGCGAGGGCGAGGGCGATGCCACCTACGGCAAGCTGAC
CCTGAAGTTCATCTGCACCACCGGCAAGCTGCCCGTGCCCTGGCCCACCCTCGTGA
CCACCCTGACCTACGGCGTGCAGTGCTTCAGCCGCTACCCCGACCACATGAAGCA
GCACGACTTCTTCAAGTCCGCCATGCCCGAAGGCTACGTCCAGGAGCGCACCATC
TTCTTCAAGGACGACGGCAACTACAAGACCCGCGCCGAGGTGAAGTTCGAGGGCG
ACACCCTGGTGAACCGCATCGAGCTGAAGGGCATCGACTTCAAGGAGGACGGCA
ACATCCTGGGGCACAAGCTGGAGTACAACTACAACAGCCACAACGTCTATATCAT
GGCCGACAAGCAGAAGAACGGCATCAAGGTGAACTTCAAGATCCGCCACAACAT
CGAGGACGGCAGCGTGCAGCTCGCCGACCACTACCAGCAGAACACCCCCATCGGC
GACGGCCCCGTGCTGCTGCCCGACAACCACTACCTGAGCACCCAGTCCGCCCTGA
GCAAAGACCCCAACGAGAAGCGCGATCACATGGTCCTGCTGGAGTTCGTGACCGC
CGCCGGGATCACTCTCGGCATGGACGAGCTGTACAAGTAA
taagcttggatccaatcaacctctggattacaaaatttgtgaaagattgactggtattcttaactatgttgctccttttacgctatgtggatacgc
tgctttaatgcctttgtatcatgctattgcttcccgtatggctttcattttctcctccttgtataaatcctggttgctgtctctttatgaggagttgtgg
cccgttgtcaggcaacgtggcgtggtgtgcactgtgtttgctgacgcaacccccactggttggggcattgccaccacctgtcagctccttt
ccgggactttcgctttccccctccctattgccacggcggaactcatcgccgcctgccttgcccgctgctggacaggggctcggctgttgg
gcactgacaattccgtggtgttgtcggggaaatcatcgtcctttccttggctgctcgcctgtgttgccacctggattctgcgcgggacgtcc
ttctgctacgtcccttcggccctcaatccagcggaccttccttcccgcggcctgctgccggctctgcggcctcttccgcgtcttcgAGA
TCTGCCTCGACTGTGCCTTCTAGTTGCCAGCCATCTGTTGTTTGCCCCTCCCCCGTG
CCTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGAACACC
AGGAGACTTGGAACGCGGAAGGGCGATATCCATCACACTGGCGGCGAATTCCCGA
TTAGGAAAGGGCGAATTCTGCAGATGGTACCACTAGTAACGGCCGCCAGTGTGCT
GGAATTCAGGctgacctcgagtttcccatgattccttcatatttgcatatacgatacaaggctgttagagagataattggaattaatt
tgactgtaaacacaaagatattagtacaaaatacgtgacgtagaaagtaataatttcttgggtagtttgcagttttaaaattatgttttaaaatg
gactatcatatgcttaccgtaacttgaaagtatttcgatttcttggctttatatatcttgtggaaaggacgaaacaccgGACTCGCGC
GAGTCGAGGAGgttttagagctagaaatagcaagttaaaataaggctagtccgttatcaacttgaaaaagtggcaccgagtcg
gtgcttttttttttagagctagaaatagcaagCTCGAGCACCTGAATTCTGCAGATATCCATCACACTG
GCGGCATCCATCACACTGGCGGCGAATTCCCGATTAGGAAAGGGCGAATTCTGCA
GATATCCATCACACTGGCGGCGAATTCCCGATAAGGATCTTCCTAGAGCATGGCT
ACGTAGATAAGTAGCATGGCGGGTTAATCATTAACTACAaggaacccctagtgatggagttggcc
actccctctctgcgcgctcgctcgctcactgaggccgggcgaccaaaggtcgcccgacgcccgggctttgcccgggcggcctcagtg
agcgagcgagcgcgcag
(SEQ ID NO: 34)
p1515_Optimized RHO HITI Donor + U6- gRNA flanked by homology arms
(Size: 5′ITR to 3′ITR: 3908 bp)
Components (5′ to 3′)
  1. 5′ inverted terminal repeats (itrs)
Ctgcgcgctcgctcgctcactgaggccgcccgggcaaagcccgggcgtcgggcgacctttggtcgcccggcctcagtgagcgagcg
agcgcgcagagagggagtggccaactccatcactaggggttcct (SEQ ID NO: 17)
  2. Additional AAV Sequences:
Tgtagttaatgattaacccgccatgctacttatctacgtagccatgctctaggaagatcggaattcactagtcaattggcggccgc (SEQ
ID NO: 64)
  3. INVERTED gRNA sequence sites(5′):
ACACCAGGAGACTTGGAACG (SEQ ID NO: 5)
  4. SpCas9 PAM 5′: CGG
  5. 5′ homology arm:
Ctccctgccgg (SEQ ID NO: 66)
  6. SPLICE ACCEPTOR SEQUENCE:
GATAGGCACCTATTGGTCTTACTGACATCCACTTTGCCTTTCTCTCCACAGGTGT
(SEQ ID NO: 9)
  7. 3XFLAG:
AGACTATAAGGACCACGACGGAGACTACAAGGATCATGATATTGATTACAAAGAC
GATGACGATAAG (SEQ ID NO: 33)
  8. cl1 degradation signal
gcctgcaagaactggttcagcagcctgagccacttcgtgatccacctg (SEQ ID NO: 6)
OPTIMIZED FURIN CLEAVAGE SITE:
CGAAAAAGAAGA (SEQ ID NO: 7)
  9. gsg linker sequence:
ggaagcgga
10. p2a ribosomal skip sequence:
gccaccaacttctccctgctgaagcaggccggcgacgtggaggagaaccccggcccc (SEQ ID NO: 1)
11. Human RHODOPSIN cDNA Sequence:
11.1 Exon1:
ATGAATGGCACAGAAGGCCCTAACTTCTACGTGCCCTTCTCCAATGCGACGGGTGT
GGTACGCAGCCCCTTCGAGTACCCACAGTACTACCTGGCTGAGCCATGGCAGTTCT
CCATGCTGGCCGCCTACATGTTTCTGCTGATCGTGCTGGGCTTCCCCATCAACTTCC
TCACGCTCTACGTCACCGTCCAGCACAAGAAGCTGCGCACGCCTCTCAACTACATC
CTGCTCAACCTAGCCGTGGCTGACCTCTTCATGGTCCTAGGTGGCTTCACCAGCACC
CTCTACACCTCTCTGCATGGATACTTCGTCTTCGGGCCCACAGGATGCAATTTGGAG
GGCTTCTTTGCCACCCTGGGCG (SEQ ID NO: 10)
11.2 Exon 2:
GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
G (SEQ ID NO: 11)
11.3 Exon 3:
GTACATCCCCGAGGGCCTGCAGTGCTCGTGTGGAATCGACTACTACACGCTCAAGC
CGGAGGTCAACAACGAGTCTTTTGTCATCTACATGTTCGTGGTCCACTTCACCATCC
CCATGATTATCATCTTTTTCTGCTATGGGCAGCTCGTCTTCACCGTCAAGGAG (SEQ
ID NO: 12)
11.4 Exon 4:
GCCGCTGCCCAGCAGCAGGAGTCAGCCACCACACAGAAGGCAGAGAAGGAGGTCA
CCCGCATGGTCATCATCATGGTCATCGCTTTCCTGATCTGCTGGGTGCCCTACGCCA
GCGTGGCATTCTACATCTTCACCCACCAGGGCTCCAACTTCGGTCCCATCTTCATGA
CCATCCCAGCGTTCTTTGCCAAGAGCGCCGCCATCTACAACCCTGTCATCTATATCA
TGATGAACAAGCAG (SEQ ID NO: 13)
11.5 Exon 5:
TTCCGGAACTGCATGCTCACCACCATCTGCTGCGGCAAGAACCCACTGGGTGACGA
TGAGGCCTCTGCTACCGTGTCCAAGACGGAGACGAGCCAGGTGGCACCAGCA (SEQ
ID NO: 14)
12. T2A Ribosomal Skip Sequence:
GGAAGCGGAGAGGGCAGAGGAAGTCTGCTAACATGCGGTGACGTCGAGGAGAAT
CCTGGACCT (SEQ ID NO: 2)
13. enhanced Green Fluorescent Protein(eGFP):
ATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGA
ACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGG
TCCTGCTGGAGTTCGTGACCGCCGCCGGGATCACTCTCGGCATGGACGAGCTGTA
CAAGTAA (SEQ ID NO: 21)
14. Sequence Unknown/Stuffer sequence:
taagcttggatcc (SEQ ID NO: 67)
15. woodchuck hepatitis virus post transcriptional regulatory element (wpre):
Taagcttggatccaatcaacctctggattacaaaatttgtgaaagattgactggtattcttaactatgttgctccttttacgctatgtggatacg
ctgctttaatgcctttgtatcatgctattgcttcccgtatggctttcattttctcctccttgtataaatcctggttgctgtctctttatgaggagttgtg
gcccgttgtcaggcaacgtggcgtggtgtgcactgtgtttgctgacgcaacccccactggttggggcattgccaccacctgtcagctcctt
tccgggactttcgctttccccctccctattgccacggcggaactcatcgccgcctgccttgcccgctgctggacaggggctcggctgttg
ggcactgacaattccgtggtgttgtcggggaaatcatcgtcctttccttggctgctcgcctgtgttgccacctggattctgcgcgggacgtc
cttctgctacgtcccttcggccctcaatccagcggaccttccttcccgcggcctgctgccggctctgcggcctcttccgcgtcttcg
(SEQ ID NO: 15)
16. Bovine Growth Hormone Poly-Adenylation Signal (BGH pA):
GCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGA (SEQ ID NO:
16)
17. 3′ homology arm
tgagaaccgc (SEQ ID NO: 68)
18. SpCas9 PAM 5′:
CGG
19. Inverted gRNA sites(5′-3′):
ACACCAGGAGACTTGGAACG (SEQ ID NO: 5)
20. Unknown sequences
Ggtaccactagtaacggccgccagtgtgctggaattcagg (SEQ ID NO: 69)
21. human u6 promoter:
Gagggcctatttcccatgattccttcatatttgcatatacgatacaaggctgttagagagataattggaattaatttgactgtaaacacaaagat
attagtacaaaatacgtgacgtagaaagtaataatttcttgggtagtttgcagttttaaaattatgttttaaaatggactatcatatgcttaccgtaa
cttgaaagtatttcgatttcttggctttatatatcttgtggaaaggacgaaacacc (SEQ ID NO: 70)
22. gRNA SEQUENCE TO INTRON1 TARGET (5′-3′):
ACACCAGGAGACTTGGAACG (SEQ ID NO: 5)
23. gRNA stuffer region
gttttagagctagaaatagcaagttaaaataaggctagtccgttatcaacttgaaaaagtggcaccgagtcggtgcttttttgttttagagctag
aaatagcaag (SEQ ID NO: 71)
24. Additional AAV sequence:
Ctcgagcacctgaattctgcagatatccatcacactggcggcatccatcacactggcggcgaattcccgattaggaaagggcgaattct
gcagatatccatcacactggcggcgaattcccgataaggatcttcctagagcatggctacgtagataagtagcatggcgggttaatcatta
actaca (SEQ ID NO: 29)
25. 3′ inverted terminal repeats (itrs)
Aggaacccctagtgatggagttggccactccctctctgcgcgctcgctcgctcactgaggccgggcgaccaaaggtcgcccgacgcc
cgggctttgcccgggcggcctcagtgagcgagcgagcgcgcag (SEQ ID NO: 26)
COMPLETE SEQUENCE (5′-3′) p1515 Optimized RHO HITI Donor + U6- gRNA flanked by
homology arms
(Size: 5′ITR to 3′ITR: 3908 bp)
ctgcgcgctcgctcgctcactgaggccgcccgggcaaagcccgggcgtcgggcgacctttggtcgcccggcctcagtgagcgagcga
gcgcgcagagagggagtggccaactccatcactaggggttccttgtagttaatgattaacccgccatgctacttatctacgtagccatgctc
taggaagatcggaattcactagtcaattggcggccgcACACCAGGAGACTTGGAACGCGGctccctgccgGAT
AGGCACCTATTGGTCTTACTGACATCCACTTTGCCTTTCTCTCCACAGGTGTAGACT
ATAAGGACCACGACGGAGACTACAAGGATCATGATATTGATTACAAAGACGATGA
CGATAAGgcctgcaagaactggttcagcagcctgagccacttcgtgatccacctgCGAAAAAGAAGAggaagcgga
gccaccaacttctccctgctgaagcaggccggcgacgtggaggagaaccccggccccATGAATGGCACAGAAGGC
CCTAACTTCTACGTGCCCTTCTCCAATGCGACGGGTGTGGTACGCAGCCCCTTCGAG
TACCCACAGTACTACCTGGCTGAGCCATGGCAGTTCTCCATGCTGGCCGCCTACAT
GTTTCTGCTGATCGTGCTGGGCTTCCCCATCAACTTCCTCACGCTCTACGTCACCGT
CCAGCACAAGAAGCTGCGCACGCCTCTCAACTACATCCTGCTCAACCTAGCCGTGG
CTGACCTCTTCATGGTCCTAGGTGGCTTCACCAGCACCCTCTACACCTCTCTGCATG
GATACTTCGTCTTCGGGCCCACAGGATGCAATTTGGAGGGCTTCTTTGCCACCCTGG
GCGGTGAAATTGCCCTGTGGTCCTTGGTGGTCCTGGCCATCGAGCGGTACGTGGTG
GTGTGTAAGCCCATGAGCAACTTCCGCTTCGGGGAGAACCATGCCATCATGGGCGT
TGCCTTCACCTGGGTCATGGCGCTGGCCTGCGCCGCACCCCCACTCGCCGGCTGGT
CCAGGTACATCCCCGAGGGCCTGCAGTGCTCGTGTGGAATCGACTACTACACGCTC
AAGCCGGAGGTCAACAACGAGTCTTTTGTCATCTACATGTTCGTGGTCCACTTCACC
ATCCCCATGATTATCATCTTTTTCTGCTATGGGCAGCTCGTCTTCACCGTCAAGGAG
GCCGCTGCCCAGCAGCAGGAGTCAGCCACCACACAGAAGGCAGAGAAGGAGGTCA
CCCGCATGGTCATCATCATGGTCATCGCTTTCCTGATCTGCTGGGTGCCCTACGCCA
GCGTGGCATTCTACATCTTCACCCACCAGGGCTCCAACTTCGGTCCCATCTTCATGA
CCATCCCAGCGTTCTTTGCCAAGAGCGCCGCCATCTACAACCCTGTCATCTATATCA
TGATGAACAAGCAGTTCCGGAACTGCATGCTCACCACCATCTGCTGCGGCAAGAAC
CCACTGGGTGACGATGAGGCCTCTGCTACCGTGTCCAAGACGGAGACGAGCCAGG
TGGCACCAGCAGGAAGCGGAGAGGGCAGAGGAAGTCTGCTAACATGCGGTGACGT
CGAGGAGAATCCTGGACCTATGGTGAGCAAGGGCGAGGAGCTGTTCACCGGGGTG
GTGCCCATCCTGGTCGAGCTGGACGGCGACGTAAACGGCCACAAGTTCAGCGTGTC
CGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTACGG
CGTGCAGTGCTTCAGCCGCTACCCCGACCACATGAAGCAGCACGACTTCTTCAAGT
CCGCCATGCCCGAAGGCTACGTCCAGGAGCGCACCATCTTCTTCAAGGACGACGGC
AACTACAAGACCCGCGCCGAGGTGAAGTTCGAGGGCGACACCCTGGTGAACCGCA
TCGAGCTGAAGGGCATCGACTTCAAGGAGGACGGCAACATCCTGGGGCACAAGCT
GGAGTACAACTACAACAGCCACAACGTCTATATCATGGCCGACAAGCAGAAGAAC
GGCATCAAGGTGAACTTCAAGATCCGCCACAACATCGAGGACGGCAGCGTGCAGC
TCGCCGACCACTACCAGCAGAACACCCCCATCGGCGACGGCCCCGTGCTGCTGCCC
GACAACCACTACCTGAGCACCCAGTCCGCCCTGAGCAAAGACCCCAACGAGAAGC
GCGATCACATGGTCCTGCTGGAGTTCGTGACCGCCGCCGGGATCACTCTCGGCATG
GACGAGCTGTACAAGTAAtaagcttggatccaatcaacctctggattacaaaatttgtgaaagattgactggtattcttaact
atgttgctccttttacgctatgtggatacgctgctttaatgcctttgtatcatgctattgcttcccgtatggctttcattttctcctccttgtataaatcc
tggttgctgtctctttatgaggagttgtggcccgttgtcaggcaacgtggcgtggtgtgcactgtgtttgctgacgcaacccccactggttgg
ggcattgccaccacctgtcagctcctttccgggactttcgctttccccctccctattgccacggcggaactcatcgccgcctgccttgcccg
ctgctggacaggggctcggctgttgggcactgacaattccgtggtgttgtcggggaaatcatcgtcctttccttggctgctcgcctgtgttgc
cacctggattctgcgcgggacgtccttctgctacgtcccttcggccctcaatccagcggaccttccttcccgcggcctgctgccggctctgc
ggcctcttccgcgtcttcgagatctGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAA
TAAAATGAGGAAATTGCATCGCATTGTCTGAGTAGGTGTCATTCTATTCTGGGGGG
TGGGGTGGGGCAGGACAGCAAGGGGGAGGATTGGGAAGACAATAGCAGGCATGC
TGGGGAtgagaaccgCGGACACCAGGAGACTTGGAACGggtaccactagtaacggccgccagtgtgctgg
aattcagggagggcctatttcccatgattccttcatatttgcatatacgatacaaggctgttagagagataattggaattaatttgactgtaaac
acaaagatattagtacaaaatacgtgacgtagaaagtaataatttcttgggtagtttgcagttttaaaattatgttttaaaatggactatcatatgc
ttaccgtaacttgaaagtatttcgatttcttggctttatatatcttgtggaaaggacgaaacaccgACACCAGGAGACTTGGA
ACGgttttagagctagaaatagcaagttaaaataaggctagtccgttatcaacttgaaaaagtggcaccgagtcggtgcttttttgttttaga
gctagaaatagcaagctcgagcacctgaattctgcagatatccatcacactggcggcatccatcacactggcggcgaattcccgattagg
aaagggcgaattctgcagatatccatcacactggcggcgaattcccgataaggatcttcctagagcatggctacgtagataagtagcatgg
cgggttaatcattaactacaaggaacccctagtgatggagttggccactccctctctgcgcgctcgctcgctcactgaggccgggcgacc
aaaggtcgcccgacgcccgggctttgcccgggcggcctcagtgagcgagcgagcgcgcag (SEQ ID NO: 72)
p1519_Optimized RHO HITI Donor + U6- scRNA flanked by homology arms
(Size: 5′ITR to 3′ITR: 3865 bp)
Components (5′ to 3′)
  1. 5′ inverted terminal repeats (itrs)
Ctgcgcgctcgctcgctcactgaggccgcccgggcaaagcccgggcgtcgggcgacctttggtcgcccggcctcagtgagcgagcg
agcgcgcagagagggagtggccaactccatcactaggggttcct (SEQ ID NO: 17)
  2. Additional AAV Sequences:
Tgtagttaatgattaacccgccatgctacttatctacgtagccatgctctaggaagatcggaattcactagtcaattggcggccgc (SEQ
ID NO: 64)
  3. INVERTED gRNA sequence sites(5′):
ACACCAGGAGACTTGGAACG (SEQ ID NO: 5)
  4. SpCas9 PAM 5′: CGG
  5. 5′ homology arm:
Ctccctgccgg (SEQ ID NO: 66)
  6. SPLICE ACCEPTOR SEQUENCE:
GATAGGCACCTATTGGTCTTACTGACATCCACTTTGCCTTTCTCTCCACAGGTGT
(SEQ ID NO: 9)
  7. 3XFLAG:
AGACTATAAGGACCACGACGGAGACTACAAGGATCATGATATTGATTACAAAGAC
GATGACGATAAG (SEQ ID NO:33)
  8. cl1 degradation signal
gcctgcaagaactggttcagcagcctgagccacttcgtgatccacctg (SEQ ID NO: 6)
OPTIMIZED FURIN CLEAVAGE SITE:
CGAAAAAGAAGA (SEQ ID NO: 7)
  9. gsg linker sequence:
ggaagcgga
10. p2a ribosomal skip sequence:
gccaccaacttctccctgctgaagcaggccggcgacgtggaggagaaccccggcccc (SEQ ID NO: 1)
11. Human RHODOPSIN cDNA Sequence:
11.1 Exon1:
ATGAATGGCACAGAAGGCCCTAACTTCTACGTGCCCTTCTCCAATGCGACGGGTGT
GGTACGCAGCCCCTTCGAGTACCCACAGTACTACCTGGCTGAGCCATGGCAGTTCT
CCATGCTGGCCGCCTACATGTTTCTGCTGATCGTGCTGGGCTTCCCCATCAACTTCC
TCACGCTCTACGTCACCGTCCAGCACAAGAAGCTGCGCACGCCTCTCAACTACATC
CTGCTCAACCTAGCCGTGGCTGACCTCTTCATGGTCCTAGGTGGCTTCACCAGCACC
CTCTACACCTCTCTGCATGGATACTTCGTCTTCGGGCCCACAGGATGCAATTTGGAG
GGCTTCTTTGCCACCCTGGGCG (SEQ ID NO: 10)
11.2 Exon 2:
GTGAAATTGCCCTGTGGTCCTTGGTGGTCCTGGCCATCGAGCGGTACGTGGTGGTG
TGTAAGCCCATGAGCAACTTCCGCTTCGGGGAGAACCATGCCATCATGGGCGTTGC
CTTCACCTGGGTCATGGCGCTGGCCTGCGCCGCACCCCCACTCGCCGGCTGGTCCA
G (SEQ ID NO: 11)
11.3 Exon 3:
GTACATCCCCGAGGGCCTGCAGTGCTCGTGTGGAATCGACTACTACACGCTCAAGC
CGGAGGTCAACAACGAGTCTTTTGTCATCTACATGTTCGTGGTCCACTTCACCATCC
CCATGATTATCATCTTTTTCTGCTATGGGCAGCTCGTCTTCACCGTCAAGGAG (SEQ
ID NO: 12)
11.4 Exon 4:
GCCGCTGCCCAGCAGCAGGAGTCAGCCACCACACAGAAGGCAGAGAAGGAGGTCA
CCCGCATGGTCATCATCATGGTCATCGCTTTCCTGATCTGCTGGGTGCCCTACGCCA
GCGTGGCATTCTACATCTTCACCCACCAGGGCTCCAACTTCGGTCCCATCTTCATGA
CCATCCCAGCGTTCTTTGCCAAGAGCGCCGCCATCTACAACCCTGTCATCTATATCA
TGATGAACAAGCAG (SEQ ID NO: 13)
11.5 Exon 5:
TTCCGGAACTGCATGCTCACCACCATCTGCTGCGGCAAGAACCCACTGGGTGACGA
TGAGGCCTCTGCTACCGTGTCCAAGACGGAGACGAGCCAGGTGGCACCAGCA (SEQ
ID NO: 14)
12. T2A Ribosomal Skip Sequence:
GGAAGCGGAGAGGGCAGAGGAAGTCTGCTAACATGCGGTGACGTCGAGGAGAAT
CCTGGACCT (SEQ ID NO: 2)
13. enhanced Green Fluorescent Protein(eGFP):
ATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGA
ACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGG
TCCTGCTGGAGTTCGTGACCGCCGCCGGGATCACTCTCGGCATGGACGAGCTGTA
CAAGTAA (SEQ ID NO: 21)
14. Sequence Unknown/Stuffer sequence:
taagcttggatcc (SEQ ID NO: 67)
15. woodchuck hepatitis virus post transcriptional regulatory element (wpre):
Taagcttggatccaatcaacctctggattacaaaatttgtgaaagattgactggtattcttaactatgttgctccttttacgctatgtggatacg
ctgctttaatgcctttgtatcatgctattgcttcccgtatggctttcattttctcctccttgtataaatcctggttgctgtctctttatgaggagttgtg
gcccgttgtcaggcaacgtggcgtggtgtgcactgtgtttgctgacgcaacccccactggttggggcattgccaccacctgtcagctcctt
tccgggactttcgctttccccctccctattgccacggcggaactcatcgccgcctgccttgcccgctgctggacaggggctcggctgttg
ggcactgacaattccgtggtgttgtcggggaaatcatcgtcctttccttggctgctcgcctgtgttgccacctggattctgcgcgggacgtc
cttctgctacgtcccttcggccctcaatccagcggaccttccttcccgcggcctgctgccggctctgcggcctcttccgcgtcttcg
(SEQ ID NO: 15)
16. Bovine Growth Hormone Poly-Adenylation Signal (BGH pA):
GCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGA (SEQ ID NO:
16)
17. 3′ homology arm
tgagaaccgc (SEQ ID NO: 68)
18. SpCas9 PAM 5′:
CGG
19. Inverted gRNA sites(5′-3′):
ACACCAGGAGACTTGGAACG (SEQ ID NO: 5)
20. Unknown sequences
Ggtaccactagtaacggccgccagtgtgctggaattcagg (SEQ ID NO: 69)
21. human u6 promoter:
Gagggcctatttcccatgattccttcatatttgcatatacgatacaaggctgttagagagataattggaattaatttgactgtaaacacaaagat
attagtacaaaatacgtgacgtagaaagtaataatttcttgggtagtttgcagttttaaaattatgttttaaaatggactatcatatgcttaccgtaa
cttgaaagtatttcgatttcttggctttatatatcttgtggaaaggacgaaacacc (SEQ ID NO: 70)
22. scRNA SEQUENCE (5′-3′):
GACTCGCGCGAGTCGAGGAG (SEQ ID NO: 24)
23.gRNA stuffer region
Gttttagagctagaaatagcaagttaaaataaggctagtccgttatcaacttgaaaaagtggcaccgagtcggtgcttttttgttttagagcta
gaaatagcaag (SEQ ID NO: 71)
24. Additional AAV sequence:
Ctcgagcacctgaattctgcagatatccatcacactggcggcatccatcacactggcggcgaattcccgattaggaaagggcgaattct
gcagatatccatcacactggcggcgaattcccgataaggatcttcctagagcatggctacgtagataagtagcatggcgggttaatcatta
actaca (SEQ ID NO: 29)
25. 3′ inverted terminal repeats (itrs)
Aggaacccctagtgatggagttggccactccctctctgcgcgctcgctcgctcactgaggccgggcgaccaaaggtcgcccgacgcc
cgggctttgcccgggcggcctcagtgagcgagcgagcgcgcag (SEQ ID NO: 26)
COMPLETE SEQUENCE (5′-3′) p1519 Optimized RHO HITI Donor + U6- scRNA flanked by
homology arms
(Size: 5′ITR to 3′ITR: 3865 bp)
ctgcgcgctcgctcgctcactgaggccgcccgggcaaagcccgggcgtcgggcgacctttggtcgcccggcctcagtgagcgagcga
gcgcgcagagagggagtggccaactccatcactaggggttccttgtagttaatgattaacccgccatgctacttatctacgtagccatgctc
taggaagatcggaattcACTAGTCAATTGGCggccgcACACCAGGAGACTTGGAACGCGGctccctg
ccgGATAGGCACCTATTGGTCTTACTGACATCCACTTTGCCTTTCTCTCCACAGGTGT
AGACTATAAGGACCACGACGGAGACTACAAGGATCATGATATTGATTACAAAGAC
GATGACGATAAGgcctgcaagaactggttcagcagcctgagccacttcgtgatccacctgCGAAAAAGAAGAgg
aagcggagccaccaacttctccctgctgaagcaggccggcgacgtggaggagaaccccggccccATGAATGGCACAGA
AGGCCCTAACTTCTACGTGCCCTTCTCCAATGCGACGGGTGTGGTACGCAGCCCCTT
CGAGTACCCACAGTACTACCTGGCTGAGCCATGGCAGTTCTCCATGCTGGCCGCCT
ACATGTTTCTGCTGATCGTGCTGGGCTTCCCCATCAACTTCCTCACGCTCTACGTCA
CCGTCCAGCACAAGAAGCTGCGCACGCCTCTCAACTACATCCTGCTCAACCTAGCC
GTGGCTGACCTCTTCATGGTCCTAGGTGGCTTCACCAGCACCCTCTACACCTCTCTG
CATGGATACTTCGTCTTCGGGCCCACAGGATGCAATTTGGAGGGCTTCTTTGCCACC
CTGGGCGGTGAAATTGCCCTGTGGTCCTTGGTGGTCCTGGCCATCGAGCGGTACGT
GGTGGTGTGTAAGCCCATGAGCAACTTCCGCTTCGGGGAGAACCATGCCATCATGG
GCGTTGCCTTCACCTGGGTCATGGCGCTGGCCTGCGCCGCACCCCCACTCGCCGGC
TGGTCCAGGTACATCCCCGAGGGCCTGCAGTGCTCGTGTGGAATCGACTACTACAC
GCTCAAGCCGGAGGTCAACAACGAGTCTTTTGTCATCTACATGTTCGTGGTCCACTT
CACCATCCCCATGATTATCATCTTTTTCTGCTATGGGCAGCTCGTCTTCACCGTCAA
GGAGGCCGCTGCCCAGCAGCAGGAGTCAGCCACCACACAGAAGGCAGAGAAGGA
GGTCACCCGCATGGTCATCATCATGGTCATCGCTTTCCTGATCTGCTGGGTGCCCTA
CGCCAGCGTGGCATTCTACATCTTCACCCACCAGGGCTCCAACTTCGGTCCCATCTT
CATGACCATCCCAGCGTTCTTTGCCAAGAGCGCCGCCATCTACAACCCTGTCATCTA
TATCATGATGAACAAGCAGTTCCGGAACTGCATGCTCACCACCATCTGCTGCGGCA
AGAACCCACTGGGTGACGATGAGGCCTCTGCTACCGTGTCCAAGACGGAGACGAG
CCAGGTGGCACCAGCAGGAAGCGGAGAGGGCAGAGGAAGTCTGCTAACATGCGGT
GACGTCGAGGAGAATCCTGGACCTATGGTGAGCAAGGGCGAGGAGCTGTTCACCG
GGGTGGTGCCCATCCTGGTCGAGCTGGACGGCGACGTAAACGGCCACAAGTTCAG
CGTGTCCGGCGAGGGCGAGGGCGATGCCACCTACGGCAAGCTGACCCTGAAGTTC
ATCTGCACCACCGGCAAGCTGCCCGTGCCCTGGCCCACCCTCGTGACCACCCTGAC
CTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGAC
GACGGCAACTACAAGACCCGCGCCGAGGTGAAGTTCGAGGGCGACACCCTGGTGA
ACCGCATCGAGCTGAAGGGCATCGACTTCAAGGAGGACGGCAACATCCTGGGGCA
CAAGCTGGAGTACAACTACAACAGCCACAACGTCTATATCATGGCCGACAAGCAG
AAGAACGGCATCAAGGTGAACTTCAAGATCCGCCACAACATCGAGGACGGCAGCG
TGCAGCTCGCCGACCACTACCAGCAGAACACCCCCATCGGCGACGGCCCCGTGCTG
CTGCCCGACAACCACTACCTGAGCACCCAGTCCGCCCTGAGCAAAGACCCCAACG
AGAAGCGCGATCACATGGTCCTGCTGGAGTTCGTGACCGCCGCCGGGATCACTCTC
GGCATGGACGAGCTGTACAAGTAATaagcttggatccaatcaacctctggattacaaaatttgtgaaagattgactg
gtattcttaactatgttgctccttttacgctatgtggatacgctgctttaatgcctttgtatcatgctattgcttcccgtatggctttcattttctcctcc
ttgtataaatcctggttgctgtctctttatgaggagttgtggcccgttgtcaggcaacgtggcgtggtgtgcactgtgtttgctgacgcaaccc
ccactggttggggcattgccaccacctgtcagctcctttccgggactttcgctttccccctccctattgccacggcggaactcatcgccgcct
gccttgcccgctgctggacaggggctcggctgttgggcactgacaattccgtggtgttgtcggggaaatcatcgtcctttccttggctgctc
gcctgtgttgccacctggattctgcgcgggacgtccttctgctacgtcccttcggccctcaatccagcggaccttccttcccgcggcctgct
gccggctctgcggcctcttccgcgtcttcgagatctGCCTCGACTGTGCCTTCTAGTTGCCAGCCATCTGT
TGTTTGCCCCTCCCCCGTGCCTTCCTTGACCCTGGAAGGTGCCACTCCCACTGTCCT
TTCCTAATAAAATGAGGAAATTGCATCGCATTGTCTGAGTAGGTGTCATTCTATTCT
GGGGGGTGGGGTGGGGCAGGACAGCAAGGGGGAGGATTGGGAAGACAATAGCAG
GCATGCTGGGGAtgagaaccgCGGACACCAGGAGACTTGGAACGggtaccactagtaacggccgcca
gtgtgctggaattcagggagggcctatttcccatgattccttcatatttgcatatacgatacaaggctgttagagagataattggaattaatttg
actgtaaacacaaagatattagtacaaaatacgtgacgtagaaagtaataatttcttgggtagtttgcagttttaaaattatgttttaaaatggac
tatcatatgcttaccgtaacttgaaagtatttcgatttcttggctttatatatcttGTGGAAAGGACGAAACACCgGACTC
GCGCGAGTCGAGGAGgttttagagctagaaatagcaagttaaaataaggctagtccgttatcaacttgaaaaagtggcaccg
agtcggtgcttttttgttttagagctagaaatagcaagctcgagcacctgaattctgcagatatccatcacactggcggcatccatcacactg
gcggcgaattcccgattaggaaagggcgaattctgcagatatccatcacactggcggcgaattcccgataaggatcttcctagagcatgg
ctacgtagataagtagcatggcgggttaatcattaactacaaggaacccctagtgatggagttggccactccctctctgcgcgctcgctcgc
tcactgaggccgggcgaccaaaggtcgcccgacgcccgggctttgcccgggcggcctcagtgagcgagcgagcgcgcag (SEQ
ID NO: 73)
```

### Definitions

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

In the present invention “at least 80% identity” means that the identity may be at least 80%, or 85% or 90% or 95% or 100% sequence identity to referred sequences. This applies to all the mentioned % of identity. In the present invention “at least 95% identity” means that the identity may be at least 95%, 96%, 97%, 98%, 99% or 100% sequence identity to referred sequences. This applies to all the mentioned % of identity. In the present invention “at least 98% identity” means that the identity may be at least 98%, 99% or 100% sequence identity to referred sequences. This applies to all the mentioned % of identity. Preferably, the % of identity relates to the full length of the referred sequence.

Included in the present invention are also nucleic acid sequences derived from the nucleotide sequences herein mentioned, e.g. functional fragments, mutants, variants, derivatives, analogues, and sequences having a % of identity of at least 80% with the sequences herein mentioned, as far as such fragments, mutants, variants, derivatives and analogues maintain the function of the sequence from which they derive.

The term <<functional>> is intended as maintaining the function of the sequence from which they derive.

The term “gene editing system” and “genome editing system” are equivalent.

### Exogenous DNA Sequences

Exogenous DNA sequences mentioned above comprise a fragment of DNA to be incorporated into genomic DNA of a target genome. In some embodiments, the exogenous DNA comprises at least a portion of a gene. The exogenous DNA may comprise a coding sequence e.g. a cDNA related to a wild type gene or to a “codon optimized” sequence for the factor that has to be expressed. In some embodiments, the exogenous DNA comprises at least an exon of a gene and/or at least one intron of a gene. In some embodiments, the exogenous DNA comprises an enhancer element or a promoter element of a gene. In some embodiments, the exogenous DNA comprises a discontinuous sequence of a gene comprising a 5′ portion of the gene fused to the 3′ portion of the gene. In some embodiments, the exogenous DNA comprises a wild type gene sequence. In some embodiments, the exogenous DNA comprises a mutated gene sequence. In some embodiments, the exogenous DNA comprises a wild type gene sequence. In some embodiments, the exogenous DNA sequence comprises a reporter gene. In some embodiments, the reporter gene is selected from at least one of a green fluorescent protein (GFP), a red fluorescent protein (RFP), a luciferase, a β-galactosidase, and a β-glucuronidase. In some embodiments, the exogenous DNA sequence comprises a gene transcription regulatory element which may e.g. comprise a promoter sequence or an enhancer sequence. In some embodiments, the exogenous DNA sequence comprises one or more exons or fragments thereof. In some embodiments, the exogenous DNA sequence comprises one or more introns or fragments thereof. In some embodiments, the exogenous DNA sequence comprises at least a portion of a 3′ untranslated region or a 5′ untranslated region. In some embodiments, the exogenous DNA sequence comprises an artificial DNA sequence. In some embodiments, the exogenous DNA sequence comprises a nuclear localization sequence and/or a nuclear export sequence. An exogenous DNA sequence, in some embodiments, comprises a segment of nucleic acid to be integrated at a target genomic locus. The exogenous DNA sequence, in some embodiments, comprises one or more polynucleotides of interest. The exogenous DNA sequence in some embodiments comprises one or more expression cassettes. Such an expression cassette, in some embodiments, comprises an exogenous DNA sequence of interest, a polynucleotide encoding a selection marker and/or a reporter gene, and regulatory components that influence expression. The exogenous DNA sequence, in some embodiments, comprises a genomic nucleic acid. The genomic nucleic acid is derived from an animal, a mouse, a human, a non-human, a rodent, a non-human, a rat, a hamster, a rabbit, a pig, a bovine, a deer, a sheep, a goat, a chicken, a cat, a dog, a ferret, a primate (e.g., marmoset, rhesus monkey), domesticated mammal or an agricultural mammal, an avian, a bacterium, an archaeon, a virus, or any other organism of interest or a combination thereof. Exogenous DNA sequences of any suitable size are integrated into a target genome. In some embodiments, the exogenous DNA sequence integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 kilobases (kb) in length. In some embodiments, the exogenous DNA sequence integrated into a genome is at least about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 (kb) in length.

### Targeting Sequences

In some embodiments, the targeting construct (which comprises the donor nucleic acid flanked at 5′ and 3′ by the inverted targeting sequences) comprises at least two targeting sequences. Targeting sequences herein are nucleic acid sequences recognized and cleaved by a nuclease. In some embodiments, the targeting sequence is about 9 to about 12 nucleotides in length, from about 12 to about 18 nucleotides in length, from about 18 to about 21 nucleotides in length, from about 21 to about 40 nucleotides in length, from about 40 to about 80 nucleotides in length, or any combination of subranges (e.g., 9-18, 9-21, 9-40, and 9-80 nucleotides). In some embodiments, the targeting sequence comprises a nuclease binding site. In some embodiments the targeting sequence comprises a nick/cleavage site. In some embodiments, the targeting sequence comprises a protospacer adjacent motif (PAM) sequence. In some embodiments, the target nucleic acid sequence (e.g., protospacer) is 20 nucleotides. In some embodiments, the target nucleic acid is less than 20 nucleotides. In some embodiments, the target nucleic acid is at least 5, 10, 15, 16, 17, 18, 19, 20, 21, 22, 23, 24, 25, 30 or more nucleotides. The target nucleic acid, in some embodiments, is at most 5, 10, 15, 16, 17, 18, 19, 20, 21, 22, 23, 24, 25, 30 or more nucleotides. In some embodiments, the target nucleic acid sequence is 16, 17, 18, 19, 20, 21, 22, or 23 bases immediately 5′ of the first nucleotide of the PAM. In some embodiments, the target nucleic acid sequence is 16, 17, 18, 19, 20, 21, 22, or 23 bases immediately 3′ of the last nucleotide of the PAM. In some embodiments, the target nucleic acid sequence is 20 bases immediately 5′ of the first nucleotide of the PAM. In some embodiments, the target nucleic acid sequence is 20 bases immediately 3′ of the last nucleotide of the PAM. In some embodiments, the target nucleic acid sequence is 5′ or 3′ of the PAM. A targeting sequence, in some embodiments includes nucleic acid sequences present in a target nucleic acid to which a nucleic acid-targeting segment of a complementary strand nucleic acid binds. For example, targeting sequences, in some embodiments, include sequences to which a complementary strand nucleic acid is designed to have base pairing. A targeting sequence in some embodiments comprises any polynucleotide, which is located, for example, in the nucleus or cytoplasm of a cell or within an organelle of a cell, such as a mitochondrion or chloroplast. Targeting sequences include cleavage sites for nucleases. A targeting sequence, in some embodiments, is adjacent to cleavage sites for nucleases. The nuclease cleaves the nucleic acid, in some embodiments, at a site within or outside of the nucleic acid sequence present in the target nucleic acid to which the nucleic acid-targeting sequence of the complementary strand binds. The cleavage site, in some embodiments, includes the position of a nucleic acid at which a nuclease produces a single-strand break or a double-strand break. For example, formation of a nuclease complex comprising a complementary strand nucleic acid hybridized to a protease recognition sequence and complexed with a protease results in cleavage of one or both strands in or near (e.g., within 1, 2, 3, 4, 5, 6, 7, 8, 9, 10, 19, 20, 23, 50, or more base pairs from) the nucleic acid sequence present in a target nucleic acid to which a spacer region of a complementary strand nucleic acid binds. The cleavage site, in some embodiments, is on only one strand or on both strands of a nucleic acid. In some embodiments, cleavage sites are at the same position on both strands of the nucleic acid (producing blunt ends) or are at different sites on each strand (producing staggered ends). Staggered ends, in some embodiments, are 5′ or 3′ overhang sticky-ends. Staggered ends, in some embodiments, are produced by sticky-end producing nucleases (e.g., Cpf1). In some embodiments, staggered ends are produced, for example, by using two nucleases, each of which produces a single-strand break at a different cleavage site on each strand, thereby producing a double-strand break. For example, a first nickase creates a single-strand break on the first strand of double-stranded DNA (dsDNA), and a second nickase creates a single-strand break on the second strand of dsDNA such that overhanging sequences are created. In some cases, the nuclease recognition sequence of the nickase on the first strand is separated from the nuclease recognition sequence of the nickase on the second strand by at least 2, 3, 4, 5, 6, 7, 8, 9, 10, 15, 20, 25, 30, 40, 50, 75, 100, 250, 500, or 1000 base pairs. Site-specific cleavage of a target nucleic acid by a nuclease, in some embodiments, occurs at locations determined by base-pairing complementarity between the complementary strand nucleic acid and the target nucleic acid. Site-specific cleavage of a target nucleic acid by a nuclease protein, in some embodiments, occurs at locations determined by a short motif, called the protospacer adjacent motif (PAM), in the target nucleic acid. For example, the PAM flanks the nuclease recognition sequence at the 3′ end of the recognition sequence. For example, the cleavage site of the nuclease, in some embodiments, is about 1 to about 25, or about 2 to about 5, or about 19 to about 23 base pairs (e.g., 3 base pairs) upstream or downstream of the PAM sequence. In some embodiments, the cleavage site of the nuclease is 3 base pairs upstream of the PAM sequence. In some embodiments, the cleavage site of the nuclease is 19 bases on the (+) strand and 23 base on the (−) strand, producing a 5′ overhang 5 nucleotides (nt) in length. In some cases, the cleavage produces blunt ends. In some cases, the cleavage produces staggered or sticky ends with 5′ overhangs. In some cases, the cleavage produces staggered or sticky ends with 3′ overhangs. Orthologs of various nuclease proteins utilize different PAM sequences. For example different Cas proteins, in some embodiments, recognize different PAM sequences. For example, in S. pyogenes, the PAM is a sequence in the target nucleic acid that comprises the sequence 5′-XRR-3′, where R is either A or G, where X is any nucleotide and X is immediately 3′ of the target nucleic acid sequence targeted by the spacer sequence. The PAM sequence of S. pyogenes Cas9 (SpyCas9) is 5′-XGG-3′, where X is any DNA nucleotide and is immediately 3′ of the nuclease recognition sequence of the non-complementary strand of the target DNA. The PAM of Cpf1 is 5′-TTX-3′, where X is any DNA nucleotide and is immediately 5′ of the nuclease recognition sequence. Preferably, The Cas9/sgRNA complex introduces DSBs 3 base pairs upstream of the PAM sequence in the genomic target sequence, resulting in two blunt ends. The exact same Cas9/sgRNA target sequence is loaded onto the donor DNA in the reverse direction. Targeted genomic loci, as well as the donor DNA, are cleaved by Cas9/gRNA and the linearized donor DNAs are integrated into target sites via the NHEJ DSB repair pathway. If donor DNA is integrated in the correct orientation, junction sequences are protected from further cleavage by Cas9/gRNA. If donor DNA integrates in the reverse orientation, Cas9/gRNA will excise the integrated donor DNA due to the presence of intact Cas9/gRNA target sites.

### Complementary Strand Nucleic Acids (Also Defined as Complementary Oligonucleotide)

A complementary strand nucleic acid, for example, a complementary strand oligonucleotide or a complementary strand RNA, refers to a nucleic acid that hybridizes to another nucleic acid, for example, the target nucleic acid in genome of a cell. A complementary strand nucleic acid may be e.g. RNA or DNA. A complementary strand nucleic acid, in some embodiments, comprises a nucleotide analog and/or a modified nucleotide. The complementary strand nucleic acid, in some embodiments, is programmed or designed to bind to a sequence of nucleic acid site-specifically. A complementary strand nucleic acid, in some embodiments, comprises one or more modifications to provide the nucleic acid with a new or enhanced feature. In some embodiments, a complementary strand nucleic acid comprises a nucleic acid affinity tag and/or synthetic nucleotide, synthetic nucleotide analog, nucleotide derivatives, and/or modified nucleotides. The complementary strand nucleic acid, in some embodiments, comprises a nucleotide sequence (e.g., a spacer), for example, at or near the 5′ end or 3′ end, that hybridizes to a sequence in a target nucleic acid. In some embodiments, the spacer of a complementary strand nucleic acid interacts with a target nucleic acid in a sequence-specific manner via hybridization (i.e., base pairing). In some embodiments, the spacer sequence hybridizes to a target nucleic acid (e.g., protospacer sequence) that is located 5′ or 3′ of protospacer adjacent motif (PAM). In some embodiments, a complementary strand nucleic acid comprises two separate nucleic acid molecules, which is referred to as a double complementary strand nucleic acid. In some embodiments, a complementary strand nucleic acid comprises a single nucleic acid molecule, which is referred to as a single complementary strand nucleic acid. In some embodiments, the complementary strand nucleic acid is a single complementary strand nucleic acid comprising a crRNA. In some embodiments, the complementary strand nucleic acid is a single complementary strand nucleic acid comprising a fused construct. The nucleic acid-targeting region of a complementary strand nucleic acid, in some embodiments, comprises a nucleotide sequence that is complementary to a sequence in a target nucleic acid. The nucleic acid-targeting region, in some embodiments, comprises the spacer region. The nucleotide sequence of a spacer region varies and determines the location within the target nucleic acid with which the complementary strand nucleic acid interacts. The spacer region of a complementary strand nucleic acid, in some embodiments, is modified to hybridize to any desired sequence within a target nucleic acid. Complementarity is alternatively perfect or substantial/sufficient. Perfect complementarity between two nucleic acids means that the two nucleic acids form a duplex in which every base in the duplex is bonded to a complementary base by Watson-Crick pairing. Substantial or sufficient complementarity means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods. In some embodiments, the nucleic acid-targeting region of a complementary strand nucleic acid (e.g., spacer region) is between 18 to 72 nucleotides in length. The nucleic acid-targeting region of a complementary strand nucleic acid (e.g., spacer region) has a length of from about 12 nucleotides to about 100 nucleotides. For example, the nucleic acid-targeting region of a complementary strand nucleic acid (e.g., spacer region) has a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, from about 12 nt to about 19 nt, from about 12 nt to about 18 nt, from about 12 nt to about 17 nt, from about 12 nt to about 16 nt, or from about 12 nt to about 15 nt. Alternatively, the DNA-targeting segment has a length of from about 18 nt to about 20 nt, from about 18 nt to about 25 nt, from about 18 nt to about 30 nt, from about 18 nt to about 35 nt, from about 18 nt to about 40 nt, from about 18 nt to about 45 nt, from about 18 nt to about 50 nt, from about 18 nt to about 60 nt, from about 18 nt to about 70 nt, from about 18 nt to about 80 nt, from about 18 nt to about 90 nt, from about 18 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

In some embodiments, the nucleic acid-targeting region of a complementary strand nucleic acid (e.g., spacer region) is 20 nucleotides in length. In some embodiments, the nucleic acid-targeting region of a complementary strand nucleic acid (e.g., spacer region) is 19 nucleotides in length. In some embodiments, the nucleic acid-targeting region of a complementary strand nucleic acid (e.g., spacer region) is 18 nucleotides in length. In some embodiments, the nucleic acid-targeting region of a complementary strand nucleic acid (e.g., spacer region) is 17 nucleotides in length. In some embodiments, the nucleic acid-targeting region of a complementary strand nucleic acid (e.g., spacer region) is 16 nucleotides in length. In some embodiments, the nucleic acid-targeting region of a complementary strand nucleic acid (e.g., spacer region) is 21 nucleotides in length. In some embodiments, the nucleic acid-targeting region of a complementary strand nucleic acid (e.g., spacer region) is 22 nucleotides in length. A protospacer sequence, in some embodiments, is identified by identifying a PAM within a region of interest and selecting a region of a desired size upstream or downstream of the PAM as the protospacer. A corresponding spacer sequence is designed by determining the complementary sequence of the protospacer region. A spacer sequence, in some embodiments, is identified using a computer program (e.g., machine readable code). The computer program, in some embodiments, uses variables such as predicted melting temperature, secondary structure formation, and predicted annealing temperature, sequence identity, genomic context, chromatin accessibility, % GC, frequency of genomic occurrence, methylation status, presence of S Ps, and the like. The percent complementarity between the nucleic acid-targeting sequence (e.g., spacer sequence) and the nuclease recognition sequence within the target nucleic acid (e.g., protospacer), in some embodiments, is at least 60%, at least 70%, at least 75%, at least 80%, at least 85%, at least 90%, at least 95%, at least 97%, at least 98%, at least 99%, or 100%. The percent complementarity between the nucleic acid-targeting sequence and the nuclease recognition sequence within the target nucleic acid, in some embodiments, is at least 60% over about 20 contiguous nucleotides. In some embodiments, complementary strand nucleic acids include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); or a modification or sequence that provides a binding site for proteins (e.g., proteins that act on DNA, including transcriptional activators, transcriptional repressors, DNA methyl transferases, DNA demethylases, histone acetyltransferases, histone deacetylases, and combinations thereof). Complementary strand nucleic acids are provided in any form, e.g. in the form of RNA, either as two molecules (e.g., separate crRNA and tracrRNA) or as one molecule (e.g., sgRNA). In some embodiments, the complementary strand nucleic acid is provided in the form of a complex with a nuclease protein. Alternatively, the complementary strand nucleic acid is also provided in the form of DNA encoding the RNA. The DNA encoding the complementary strand nucleic acid alternatively encodes a single complementary strand nucleic acid (e.g., sgRNA) or separate RNA molecules (e.g., separate crRNA and tracrRNA). In the latter case, the DNA encoding the complementary strand nucleic acid is provided as separate DNA molecules encoding the crRNA and tracrRNA, respectively. In some embodiments, DNAs encoding complementary strand nucleic acid are stably integrated in the genome of the cell and, optionally, operably linked to a promoter active in the cell. DNAs encoding complementary strand nucleic acids, in some embodiments, are operably linked to a promoter in an expression construct. Complementary strand nucleic acids are prepared by any suitable method. For example, complementary strand nucleic acids are prepared by in vitro transcription using, for example, T7 RNA polymerase. In some embodiments, complementary strand nucleic acids are also synthetically produced molecules prepared by chemical synthesis.

### Nucleases

Nucleases recognizing a targeting sequence are known by those of skill in the art and include, but are not limited to, zinc finger nucleases (ZFN), transcription activator-like effector nucleases (TALEN), clustered regularly interspaced short palindromic repeats (CRISPR) nucleases, and meganucleases. Nucleases found in compositions and useful in methods disclosed herein are described in more detail below.

### Zinc Finger Nucleases (ZFNs)

“Zinc finger nucleases” or “ZFNs” are a fusion between the cleavage domain of Fokl and a DNA recognition domain containing 3 or more zinc finger motifs. The heterodimerization at a particular position in the DNA of two individual ZFNs in precise orientation and spacing leads to a double-strand break in the DNA. In some cases, ZFNs fuse a cleavage domain to the C-terminus of each zinc finger domain. In order to allow the two cleavage domains to dimerize and cleave DNA, the two individual ZFNs bind opposite strands of DNA with their C-termini at a certain distance apart. In some cases, linker sequences between the zinc finger domain and the cleavage domain require the 5′ edge of each binding site to be separated by about 5-7 bp. Exemplary ZFNs that are useful in the present invention include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140. In some embodiments, a ZFN is a zinc finger nickase which, in some embodiments, is an engineered ZFN that induces site-specific single-strand DNA breaks or nicks. Descriptions of zinc finger nickases are found, e.g., in Ramirez et al., Nucl Acids Res, 2012, 40(12):5560-8; Kim et al., Genome Res, 2012, 22(7): 1327-33.

### TALENs

“TALENs” or “TAL-effector nucleases” are engineered transcription activator-like effector nucleases that contain a central domain of DNA-binding tandem repeats, a nuclear localization signal, and a C-terminal transcriptional activation domain. In some instances, a DNA-binding tandem repeat comprises 33-35 amino acids in length and contains two hypervariable amino acid residues at positions 12 and 13 that recognize one or more specific DNA base pairs. TALENs are produced by fusing a TAL effector DNA binding domain to a DNA cleavage domain. For instance, a TALE protein may be fused to a nuclease such as a wild-type or mutated Fokl endonuclease or the catalytic domain of Fokl. Several mutations to Fokl have been made for its use in TALENs, which, for example, improve cleavage specificity or activity. Such TALENs are engineered to bind any desired DNA sequence. TALENs are often used to generate gene modifications by creating a double-strand break in a target DNA sequence, which in turn, undergoes NHEJ or HDR. In some cases, a single-stranded donor DNA repair template is provided to promote HDR. Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4: 1762; and Joung and Sander, Nat Rev Mol Cell Biol, 2013, 14(1):49-55. DNA

### Guided Nucleases

“DNA guided nucleases” are nucleases that use a single stranded DNA complementary nucleotide to direct the nuclease to the correct place in the genome by hybridizing to another nucleic acid, for example, the target nucleic acid in the genome of a cell. In some embodiments, the DNA guided nuclease comprises an Argonaute nuclease. In some embodiments, the DNA guided nuclease is selected from TtAgo, PfAgo, and NgAgo. In some embodiments, the DNA guided nuclease is NgAgo.

### Meganucleases

“Meganucleases” are rare-cutting endonucleases or homing endonucleases that, in certain embodiments, are highly specific, recognizing DNA target sites ranging from at least 12 base pairs in length, e.g., from 12 to 40 base pairs or 12 to 60 base pairs in length.

Any meganuclease is contemplated to be used herein, including, but not limited to, I-Scel, I-Scell, I-SceIII, I-SceIV, I-SceV, I-SceVI, I-SceVII, I-Ceul, I-CeuAIIP, I-Crel, I-CrepsblP, I-CrepsbllP, I-CrepsbIIIP, I-CrepsbIVP, I-Tlil, I-Ppol, PI-PspI, F-Scel, F-Scell, F-Suvl, F-Tevl, F-TevII, I-Amal, I-Anil, I-Chul, I-Cmoel, I-Cpal, I-CpaII, I-Csml, I-Cvul, I-CvuAIP, I-Ddil, I-DdiII, I-Dirl, I-Dmol, I-Hmul, I-HmuII, I-HsNIP, I-Llal, I-Msol, I-Naal, I-Nanl, I-NcIIP, I-NgrIP, I-Nitl, I-Njal, I-Nsp236IP, I-Pakl, I-PboIP, I-PcuIP, I-PcuAI, I-PcuVI, I-PgrlP, 1-PobIP, I-Porl, I-PorIIP, I-PbpIP, I-SpBetaIP, I-Scal, I-SexIP, 1-SneIP, I-Spoml, I-SpomCP, I-SpomIP, I-SpomIIP, I-SquIP, I-Ssp6803I, I-SthPhiJP, I-SthPhiST3P, I-SthPhiSTe3bP, I-TdeIP, I-Tevl, I-TevII, I-TevIII, I-UarAP, I-UarHGPAIP, I-UarHGPA13P, I-VinlP, 1-ZbiIP, PI-MtuI, PI-MtuHIP PI-MtuHIIP, PI-PfuI, PI-PfuII, PI-PkoI, Pl-PkoII, PI-Rma43812IP, PI-SpBetaIP, PI-SceI, PI-Tful, PI-TfuII, PI-Thyl, PI-Tlil, PI-THII, I-Crel meganuclease, I-Ceul meganuclease, I-Msol meganuclease, I-Scel meganuclease, or any active variants, fragments, mutants or derivatives thereof

### CRISPR

The CRISPR (Clustered Regularly Interspaced Short Palindromic Repeats)/Cas (CRISPR-associated protein) nuclease system is an engineered nuclease system based on a bacterial system that is used for genome engineering. It is based in part on the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide complementary strand sequence contained within the crRNA transcript. The Cas (e.g., Cas9) nuclease, in some embodiments, requires both the crRNA and the tracrRNA for site-specific DNA recognition and cleavage. This system has now been engineered such that, in certain embodiments, the crRNA and tracrRNA are combined into one molecule (the “single guide RNA” or “sgRNA”), and the crRNA equivalent portion of the single guide RNA is engineered to guide the Cas (e.g., Cas9) nuclease to target any desired sequence (see, e.g., Jinek et al. (2012) Science 337:816-821; Jinek et al. (2013) eLife 2:e00471; Segal (2013) eLife 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ). In some embodiments, the Cas nuclease has DNA cleavage activity. The Cas nuclease, in some embodiments, directs cleavage of one or both strands at a location in a target DNA sequence. For example, in some embodiments, the Cas nuclease is a nickase having one or more inactivated catalytic domains that cleaves a single strand of a target DNA sequence. Non-limiting examples of Cas nucleases include Cas1, Cas1B, Cas2, Cas3, Cas4, Cas5, Cas6, Cas7, Cas8, Cas9 (also known as Csn1 and Csx12), Cas1O, Cpf1, C2c3, C2c2 and C2c1Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx1O, Csx16, CsaX, Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include, but are not limited to, Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref Seq. No. NP 269215, and the amino acid sequence of Streptococcus thermophilus wild-type Cas9 polypeptide is set forth, e.g., in NBCI Ref. Seq. No. WP_011681470. Cas nucleases, e.g., Cas9 polypeptides, in some embodiments, are derived from a variety of bacterial species. “Cas9” refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme, in some embodiments, comprises one or more catalytic domains of a Cas9 protein derived from bacteria belonging to the group consisting of Corynebacter, Sutterella, Legionella, Treponema, Filif actor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g. the two catalytic domains are derived from different bacteria species. Useful variants of the Cas9 nuclease include a single inactive catalytic domain, such as a RuvC- or HNH-enzyme or a nickase. A Cas9 nickase has only one active functional domain and, in some embodiments, cuts only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863 A. A double-strand break is introduced using a Cas9 nickase if at least two DNA-targeting RNAs that target opposite DNA strands are used. A double-nicked induced double-strand break is repaired by NHEJ or HDR. This gene editing strategy favors HDR and decreases the frequency of indel mutations at off-target DNA sites. The Cas9 nuclease or nickase, in some embodiments, is codon-optimized for the target cell or target organism. In some embodiments, the Cas nuclease is a Cas9 polypeptide that contains two silencing mutations of the RuvCl and HNH nuclease domains (D10A and H840A), which is referred to as dCas9. In one embodiment, the dCas9 polypeptide from Streptococcus pyogenes comprises at least one mutation at position D10, G12, G17, E762, H840, N854, N863, H982, H983, A984, D986, A987, or any combination thereof. Descriptions of such dCas9 polypeptides and variants thereof are provided in, for example, International Patent Publication No. WO 2013/176772. The dCas9 enzyme in some embodiments, contains a mutation at D10, E762, H983, or D986, as well as a mutation at H840 or N863. In some instances, the dCas9 enzyme contains a D10A or DION mutation. Also, the dCas9 enzyme alternatively includes a mutation H840A, H840Y, or H840N. In some embodiments, the dCas9 enzyme of the present invention comprises D10A and H840A; D10A and H840Y; D10A and H840N; DION and H840A; DION and H840Y; or DION and H840N substitutions. The substitutions are alternatively conservative or non-conservative substitutions to render the Cas9 polypeptide catalytically inactive and able to bind to target DNA. For genome editing methods, the Cas nuclease in some embodiments comprises a Cas9 fusion protein such as a polypeptide comprising the catalytic domain of the type IIS restriction enzyme, Fokl, linked to dCas9. The FokI-dCas9 fusion protein (fCas9) can use two guide RNAs to bind to a single strand of target DNA to generate a double-strand break.

### Delivery

The gene delivery vehicles of the present invention may be administered to a patient. Said administration may be an “in vivo” administration or an “ex vivo” administration. A skilled worker would be able to determine appropriate dosage rates. The term “administered” includes delivery by viral or non-viral techniques.

Non-viral vectors are a heterogeneous group of delivery vectors that comprise polyplexes, lipid nanoparticles, non-lipid nanoparticles, virus-like particles or combinations of these. In comparison with viral vectors, this group is characterized by low cytotoxic, immunogenic and mutagenic profiles. Moreover, they also present high cargo capacity (Zu & Gao (2021) APPS J. 23-78).

Most lipids consist of positively charged headgroups which bind with the anionic phosphate groups of nucleic acids via electrostatic interactions to form lipid nanoparticles.

Polyplexes are formed when polyanionic nucleic acids compact with polycationic polymers.

Non-lipid nanoparticles may comprise carbon- or metal-based nanoparticles, examples of which include carbon nanotubes, graphene or carbon quantum dots (CQDs) and gold or iron oxide nanoparticles.

Virus-like particles are virus-derived structures made of one or more different molecules with the ability to self-assemble, mimicking the form and size of a viral particle, therefore they maintain the ability to transduce the target cell, but they lack the viral genetic material (Nooraei et al (2021) Journal of Nanobiotechnology, 19-59).

Viral delivery mechanisms include but are not limited to adenoviral vectors, adeno-associated viral (AAV) vectors, herpes viral vectors, retroviral vectors, lentiviral vectors, integrase-defective lentiviral vectors and baculoviral vectors etc as described above. Non-viral delivery systems include DNA transfection such as electroporation, lipid mediated transfection, compacted DNA-mediated transfection; liposomes, immunoliposomes, lipofectin, cationic facial amphiphiles (CFAs) and combinations thereof. The delivery of one or more therapeutic genes by a vector system according to the present invention may be used alone or in combination with other treatments or components of the treatment.

Any suitable delivery method is contemplated to be used for delivering the compositions of the disclosure. The individual components of the HITI system (e.g., nuclease and/or the exogenous DNA sequence), in some embodiments, are delivered simultaneously or temporally separated. The choice of method of genetic modification is dependent on the type of cell being transformed and/or the circumstances under which the transformation is taking place (e.g., in vitro, ex vivo, or in vivo). A general discussion of these methods is found in Ausubel, et al., Short Protocols in Molecular Biology, 3rd ed., Wiley & Sons, 1995.

The term “contacting the cell” comprises all the delivery method herein discloses. In some embodiments, a method as disclosed herein involves contacting a target DNA or introducing into a cell (or a population of cells) one or more nucleic acids comprising nucleotide sequences encoding a complementary strand nucleic acid (e.g., gRNA), a site-directed modifying polypeptide (e.g., Cas protein), and/or a exogenous DNA sequence. Suitable nucleic acids comprising nucleotide sequences encoding a complementary strand nucleic acid and/or a site-directed modifying polypeptide include expression vectors, where an expression vector comprising a nucleotide sequence encoding a complementary strand nucleic acid and/or a site-directed modifying polypeptide is a recombinant expression vector. Non-limiting examples of delivery methods or transformation include, for example, viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, and nanoparticle-mediated nucleic acid delivery (see, e.g., Panyam et., al Adv Drug Deliv Rev. 2012 Sep. 13. pii: 50169-409X(12)00283-9. doi: 10.1016/j .addr.2012.09.023). In some aspects, the present invention provides methods comprising delivering one or more polynucleotides, such as or one or more vectors as described herein, one or more transcripts thereof, and/or one or proteins transcribed therefrom, to a host cell. In some aspects, the disclosure further provides cells produced by such methods, and organisms (such as animals, plants, or fungi) comprising or produced from such cells. In some embodiments, a nuclease protein in combination with, and optionally complexed with, a complementary strand sequence is delivered to a cell. Conventional viral and non-viral based gene transfer methods are contemplated to be used to introduce nucleic acids in mammalian cells or target tissues. Such methods are used to administer nucleic acids encoding components of a HITI system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems can include DNA and RNA viruses, which can have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11: 162-166 (1993); Dillon. TIBTECH 11: 167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1: 13-26 (1994). Methods of non-viral delivery of nucleic acids can include lipofection, nucleofection, microinjection, electroporation, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery is contemplated to be to cells (e.g. in vitro or ex vivo administration) or target tissues (e.g. in vivo administration). The preparation of lipid:nucleic acid complexes, including targeted liposomes such as immunolipid complexes, is well known (see, e.g., Crystal, Science 270:404-410 (1995); Blaese et al., Cancer Gene Ther. 2:291-297 (1995): Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787). RNA or DNA viral based systems are used to target specific cells in the body and trafficking the viral payload to the nucleus of the cell. Viral vectors are alternatively administered directly (in vivo) or they are used to treat cells in vitro, and the modified cells are optionally be administered (ex vivo). Viral based systems include, but are not limited to, retroviral, lentivirus, adenoviral, adeno-associated, and herpes simplex virus vectors for gene transfer. Integration in the host genome, in some embodiments, occurs with the retrovirus, lentivirus, and adeno-associated virus gene transfer methods, which results in long term expression of the inserted transgene, in some embodiments. High transduction efficiencies are observed in many different cell types and target tissues. In some embodiments, adenoviral-based systems are used. Adenoviral-based systems, in some embodiments, lead to transient expression of the transgene. Adenoviral based vectors are capable of high transduction efficiency in cells and in some embodiments do not require cell division. High titer and levels of expression are possible with adenoviral based vectors. In some embodiments, adeno-associated virus (“AAV”) vectors are used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94: 1351 (1994). Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Packaging cells, in some embodiments, are used to form virus particles capable of infecting a host cell. Such cells include but are not limited to 293 cells, (e.g., for packaging adenovirus), and .psi.2 cells or PA317 cells (e.g., for packaging retrovirus). Viral vectors are generated by producing a cell line that packages a nucleic acid vector into a viral particle. In some cases, the vectors contain the minimal viral sequences required for packaging and subsequent integration into a host. In some cases, the vectors contain other viral sequences being replaced by an expression cassette for the polynucleotide(s) to be expressed. In some embodiments, the missing viral functions are supplied in trans by the packaging cell line. For example, in some embodiments, AAV vectors comprise ITR sequences from the AAV genome which are required for packaging and integration into the host genome. Viral DNA is packaged in a cell line, which contains a helper plasmid encoding the other AAV genes, namely rep and cap, while lacking ITR sequences. Alternatively, the cell line is infected with adenovirus as a helper. The helper virus promotes replication of the AAV vector and expression of AAV genes from the helper plasmid. Contamination with adenovirus is reduced by, e.g., heat treatment, to which adenovirus is more sensitive than AAV.

### AAV Serotypes

To date, dozens of different AAV variants (serotypes) have been identified and classified (Srivastava A, Curr Opin Virol. 2016 December; 21:75-80). All of the known serotypes can infect cells from multiple diverse tissue types. Tissue specificity is determined by the capsid serotype and pseudotyping of AAV vectors to alter their tropism range will likely be important to their use in therapy. Pseudotyped AAV vectors are those which contain the genome of one AAV serotype in the capsid of a second AAV serotype; for example an AAV2/8 vector contains the AAV8 capsid and the AAV 2 genome (Auricchio et al. (2001) Hum. Mol. Genet. 10(26):3075-81). Such vectors are also known as chimeric vectors

### Serotype 2

Serotype 2 (AAV2) has been the most extensively examined so far. AAV2 presents natural tropism towards neurons, vascular smooth muscle cells and hepatocytes. Three cell receptors have been described for AAV2: heparan sulfate proteoglycan (HSPG), αᵥβ₅ integrin and fibroblast growth factor receptor 1 (FGFR-1). The first functions as a primary receptor, while the latter two have a co-receptor activity and enable AAV to enter the cell by receptor-mediated endocytosis. These study results have been disputed by Qiu, Handa, et al., HSPG functions as the primary receptor, though its abundance in the extracellular matrix can scavenge AAV particles and impair the infection efficiency.

### Other Serotypes

Although AAV2 is the most popular serotype in various AAV-based research, it has been shown that other serotypes can be more effective as gene delivery vectors. For instance AAV6 appears much better in infecting airway epithelial cells, AAV7 presents very high transduction rate of murine skeletal muscle cells (similarly to AAV1 and AAV5), AAV8 is superb in transducing hepatocytes and photorecetors, AAV1 and 5 were shown to be very efficient in gene delivery to vascular endothelial cells. In the brain, most AAV serotypes show neuronal tropism, while AAV5 also transduces astrocytes. AAV6, a hybrid of AAV1 and AAV2, also shows lower immunogenicity than AAV2. Serotypes can differ with the respect to the receptors they are bound to. For example AAV4 and AAV5 transduction can be inhibited by soluble sialic acids (of different form for each of these serotypes), and AAV5 was shown to enter cells via the platelet-derived growth factor receptor. Novel AAV variants such as quadruple tyrosine mutants or AAV 2/7m8 were shown to transduce the outer retina from the vitreous in small animal models (Dalkara D et al., Sci Transl Med. 2013 Jun. 12; 5(189):189ra76; Petrs-Silva H et al., Mol Ther. 2011 February; 19(2):293-301). Another AAV mutant named ShH10, an AAV6 variant with improved glial tropism after intravitreal administration (Klimczak R R et al., PLoS One. 2009 Oct. 14; 4(10):e7467.). A further AAV mutant with particularly advantageous tropism for the retina is the AAV2 (quad Y-F) (Hickey D G et al., Gene Ther. 2017 December; 24(12):787-800). Within the meaning of the present invention, an AAV viral particle comprises capsid proteins of an AAV of a serotype selected from one or more of the group consisting of AAV1, AAV2, AAV3, AAV4, AAV5, AAV6, AAV7, AAV8 AAV9 and AAV 10, preferably from the AAV2 or AAV8 serotype.

Any suitable vector compatible with the host cell is contemplated to be used with the methods of the invention. Non-limiting examples of vectors for eukaryotic host cells include pXT1, pSG5, pSVK3, pBPV, pMSG, and pSVLSV40. In some embodiments, a nucleotide sequence encoding a complementary strand nucleic acid and/or a site-directed modifying polypeptide is operably linked to a control element, e.g., a transcriptional control element, such as a promoter. The transcriptional control element is functional, in some embodiments, in either a eukaryotic cell, e.g., a mammalian cell, or a prokaryotic cell (e.g., bacterial or archaeal cell). In some embodiments, a nucleotide sequence encoding a complementary strand nucleic acid and/or a site-directed modifying polypeptide is operably linked to multiple control elements that allow expression of the nucleotide sequence encoding a complementary strand nucleic acid and/or a site-directed modifying polypeptide in prokaryotic and/or eukaryotic cells. Depending on the host/vector system utilized, any of a number of suitable transcription and translation control elements, including constitutive and inducible promoters, transcription enhancer elements, transcription terminators, etc. may be used in the expression vector (e.g., U6 promoter, HI promoter, etc.; see above) (see e.g., Bitter et al. (1987) Methods in Enzymology, 153:516-544). In some embodiments, a complementary strand nucleic acid and/or a site-directed modifying polypeptide is provided as RNA. In such cases, the complementary strand nucleic acid and/or the RNA encoding the site-directed modifying polypeptide is produced by direct chemical synthesis or may be transcribed in vitro from a DNA encoding the complementary strand nucleic acid. The complementary strand nucleic acid and/or the RNA encoding the site-directed modifying polypeptide are synthesized in vitro using an RNA polymerase enzyme (e.g., T7 polymerase, T3 polymerase, SP6 polymerase, etc.). Once synthesized, the RNA directly contacts a target DNA or is introduced into a cell using any suitable technique for introducing nucleic acids into cells (e.g., microinjection, electroporation, transfection, etc). Nucleotides encoding a complementary strand nucleic acid (introduced either as DNA or RNA) and/or a site-directed modifying polypeptide (introduced as DNA or RNA) and/or an exogenous DNA sequence are provided to the cells using a suitable transfection technique; see, e.g. Angel and Yanik (2010) PLoS ONE 5(7): e11756, and the commercially available TransMessenger® reagents from Qiagen, Stemfect™ RNA Transfection Kit from Stemgent, and TransIT®-mRNA Transfection Kit from Minis Bio LLC. Nucleic acids encoding a complementary strand nucleic acid and/or a site-directed modifying polypeptide and/or a chimeric site-directed modifying polypeptide and/or an exogenous DNA sequence may be provided on DNA vectors. Many vectors, e.g., plasmids, cosmids, minicircles, phage, viruses, etc., useful for transferring nucleic acids into target cells are available. The vectors comprising the nucleic acid(s) in some embodiments are maintained episomally, e.g. as plasmids, minicircle DNAs, viruses such cytomegalovirus, adenovirus, etc., or they are integrated into the target cell genome, through homologous recombination or random integration, e.g. retrovirus-derived vectors such as MMLV, HIV-1, and ALV.

Also an AAV serotype derivative can be used. A serotype derivative can be obtained with three major approaches to capsid modifications: natural diversity, directed evolution, and mutants. Natural primate AAV diversity includes the major and unique AAV clades. Directed evolution begins with parental serotypes, and these are diversified via recombination-based techniques (Viruses. 2021 July; 13(7): 1336. Adeno-Associated Virus (AAV) Gene Delivery: Dissecting Molecular Interactions upon Cell Entry. Edward E. Large, Mark A. Silveria, Grant M. Zane, Onellah Weerakoon, and Michael S. Chapman)

### Methods of Making Changes to Genomic DNA

Provided herein are homology-independent targeted integration (HITI) and microhomology-mediated end joining (MMEJ) methods and compositions for making changes to nucleic acid, such as genomic DNA, including genomic DNA in non-dividing or terminally differentiated cells that do not divide. The cells herein mentioned are preferably non-diving cells, more preferably terminally differentiated cells or quiescent cells. Methods herein, at least in some embodiments, are homology independent, using non-homologous end-joining to insert exogenous DNA into a target DNA, such as a genomic DNA of a cell, such as a non-dividing or terminally differentiated cell. In some embodiments, methods herein comprise a method of integrating an exogenous DNA sequence into a genome of a non-dividing cell comprising contacting the non-dividing cell with a composition comprising a targeting construct comprising the exogenous DNA sequence and a targeting sequence, an oligonucleotide complementary to the targeting sequence, and a nuclease, wherein the exogenous DNA sequence comprises at least one nucleotide difference compared to the genome and the targeting sequence is recognized by the nuclease. In some embodiments of HITI methods disclosed herein, exogenous DNA sequences are fragments of DNA containing the desired sequence to be inserted into the genome of the target cell or host cell. At least a portion of the exogenous DNA sequence has a sequence homologous to a portion of the genome of the target cell or host cell and at least a portion of the exogenous DNA sequence has a sequence not homologous to a portion of the genome of the target cell or host cell. For example, in some embodiments, the exogenous DNA sequence may comprise a portion of a host cell genomic DNA sequence with a mutation therein. Therefore, when the exogenous DNA sequence is integrated into the genome of the host cell or target cell, the mutation found in the exogenous DNA sequence is carried into the host cell or target cell genome. In some embodiments of HITI methods disclosed herein, the exogenous DNA sequence is flanked by at least one targeting sequence. In some embodiments, the exogenous DNA sequence is flanked by two targeting sequences. The targeting sequence comprises a specific DNA sequence that is recognized by at least one nuclease. In some embodiments, the targeting sequence is recognized by the nuclease in the presence of a oligonucleotide complementary to the targeting sequence. In some embodiments, in HITI methods disclosed herein, a targeting sequence comprises a nucleotide sequence that is recognized and cleaved by a nuclease. Nucleases recognizing a targeting sequence are known by those of skill in the art and include but are not limited to zinc finger nucleases (ZFN), transcription activator-like effector nucleases (TALEN), and clustered regularly interspaced short palindromic repeats (CRISPR) nucleases. ZFNs, in some embodiments, comprise a zinc finger DNA-binding domain and a DNA cleavage domain, fused together to create a sequence specific nuclease. TALENs, in some embodiments, comprise a TAL effector DNA binding domain and a DNA cleavage domain, fused together to create a sequence specific nuclease. CRISPR nucleases, in some embodiments, are naturally occurring nucleases that recognize DNA sequences homologous to clustered regularly interspaced short palindromic repeats, commonly found in prokaryotic DNA. CRISPR nucleases include, but are not limited to, Cas9 Cpf1, C2c3, C2c2, and C2c1. Conveniently, a Cas 9 of the present invention is a variant with reduced off target activity as SpCas9 D10A (Ran, F. A., et al., Genome engineering using the CRISPR-Cas9 system. Nat Protoc, 2013. 8(11): p. 2281-2308. (with Inactivation of RuvC domain cleavage activity), SpCas9 N863A (Ran, F. A., et al., Genome engineering using the CRISPR-Cas9 system. Nat Protoc, 2013. 8(11): p. 2281-2308) (Inactivation of HNH domain cleavage activity), SpCas9-HF1 (Kleinstiver, B. P., et al., High-fidelity CRISPR-Cas9 nucleases with no detectable genome-wide off-target effects. Nature, 2016. 529(7587): p. 490-5) (Reduction of Cas9 binding energy by protein engineering), eSpCas9 (laymaker, I. M., et al., Rationally engineered Cas9 nucleases with improved specificity. Science, 2016. 351(6268): p. 84-8) (Reduction of positive charge of Cas9), EvoCas9 (asini, A., et al., A highly specific SpCas9 variant is identified by in vivo screening in yeast. Nat Biotechnol, 2018. 36(3): p. 265-271) (Mutagenesis of REC3 domain), KamiCas9 (Merienne, N., et al., The Self-Inactivating KamiCas9 System for the Editing of CNS Disease Genes. Cell Rep, 2017. 20(12): p. 2980-2991) (Knockout of Cas9 after expression)

HITI and MMEJ methods disclosed herein, in some embodiments, are capable of introducing mutations into a host genome or a target genome as well as repairing mutations in a host genome or a target genome. Mutations or wild-type sequences, in some embodiments of the methods described herein, are found in the exogenous DNA sequence to be inserted into the host genome or target genome. Mutations are known by those of skill in the art and include single base-pair changes or point mutations, insertions, and deletions. In some embodiments, a single base-pair change results in a missense mutation which creates a codon that encodes a different amino acid in transcribed mRNA than the wild-type sequence. In some embodiments, a single base-pair change results in a nonsense mutation which encodes for a stop codon in transcribed mRNA. In some embodiments, a stop codon in transcribed RNA results in early truncation of a protein translated from the mRNA. In some embodiments, a single base-pair change results in a silent mutation that does not result in any change in amino acids encoded by a mRNA transcribed from the host genome or the target genome. In some embodiments, a silent mutation is in an intron. In some embodiments, a silent mutation is in an exon and creates a codon encoding for the same amino acid as the wild-type sequence. In some embodiments, a silent mutation, is in a promoter, an enhancer, a 5′ UTR, a 3′ UTR, or other non-coding region of the host genome or target genome. In some embodiments, a silent mutation results in aberrant splicing of an mRNA transcript. In some embodiments, a silent mutation disrupts a RNA splice donor or splice acceptor sequence. In some embodiments, a silent mutation results in aberrant RNA export. In some embodiments, a silent mutation results in aberrant or reduced translation of an mRNA. In some embodiments, a silent mutation results in aberrant or reduced transcription of an RNA. In some embodiments, mutations comprise insertions into the host genome or target genome. In some embodiments, insertions comprise a specific number of nucleotides ranging from 1 to 4,700 base pairs, for example 1-10, 5-20, 15-30, 20-50, 40-80, 50-100, 100-1000, 500-2000, 1000-4,700 base pairs. In some embodiments, the method comprises eliminating at least one gene, or fragment thereof, from the host genome or target genome. In some embodiments, the method comprises introducing an exogenous gene (herein also defined as Eexogenous DNA sequence or gene of interest), or fragment thereof, into the host genome or target genome. In some embodiments, the method comprises replacing a mutated gene, or fragment thereof, in the host genome or target genome with a wild-type gene, or fragment thereof. In some embodiments the host gene is silenced and replaced by a wild-type gene or coding sequence thereof. In some embodiments, the method changes at least one nucleotide of a host genome or target genome resulting in increased expression of a gene. In some embodiments, the method changes at least one nucleotide of a host genome or target genome resulting in decreased expression of a gene. In some embodiments, the method introduces an exogenous promoter into the host genome or target genome resulting in altered expression of a gene. In some embodiments, the promoter is an inducible promoter. HITI methods disclosed herein have increased capabilities in making changes to genomic DNA in non-dividing cells. Non-dividing cells include, but are not limited to: retinal cells, preferably retinal ganglion cells, bipolar cells, amacrine cells, retinal pigment epithelium, horizontal cells, rods and cones cells or cells of the anterior region of the eye such as iris pigment epithelium, corneal epithelium, corneal fibroblasts, cells in the central nervous system including neurons, oligodendrocytes, microglia and ependymal cells; sensory transducer cells; autonomic neuron cells; sense organ and peripheral neuron supporting cells; cells in the retina including photoreceptors, rods and cones; cells in the kidney including parietal cells, glomerulus podocytes, proximal tubule brush border cells, loop of henle thin segment cells, distal tubule cells, collecting duct cells; cells in the hematopoietic lineage including lymphocytes, monocytes, neutrophils, eosinophils, basophils, thrombocytes; cells of liver including hepatocytes, stellate cells, the Kupffer cells and the liver endothelial cells; pancreatic endocrine cells including alpha, beta, delta, gamma, and epsilon cells; cells of the respiratory epithelium including ciliated cells, basal cells, goblet cells and alveolar cells, germ cells including oogonium/oocyte, spermatid, spermatocyte, spermatogonium cell and spermatozoon; cells of the bone including osteocytes, osteoclasts and osteoblasts; cells of the heart including cardiomyocytes and cardiac pacemaker cells; follicular cells in the thyroid; cells in the upper digestive tract including serous cells, mucous cells and taste buds; cells in the stomach including parietal cells, chief cells, enteroendocrine cells; endothelial cells, epithelial cells, adipocytes, bone marrow cells, inner ear cells, dermis cells, smooth muscle cells, skeletal muscle cells. In some embodiments, HITI methods disclosed herein provide a method of making changes to genomic DNA in dividing cells, wherein the method has higher efficiency than previous methods disclosed in the art. Dividing cells include, but are not limited to, hematopoietic stem cells, mesenchymal stem cells, neural stem cells, liver stem cells, muscle satellite cells, epidermis cells, glial cells, and astrocytes. In some embodiments, the targeting construct, the complementary strand oligonucleotides, and/or a polynucleotide encoding the nuclease for HITI methods described herein are introduced into the target cell or the host cell by a virus. Viruses, in some embodiments, infect the target cell and express the targeting construct, the complementary strand oligonucleotides, and the nuclease, which allows the exogenous DNA of the targeting construct to be integrated into the host genome. In some embodiments, the virus comprises a sendai virus, a retrovirus, a lentivirus, a baculovirus, an adenovirus, or an adeno-associated virus. In some embodiments the virus is a pseudotyped virus. In some embodiments, the targeting construct, the complementary strand oligonucleotides, and/or a polynucleotide encoding the nuclease for HITI methods described herein are introduced into the target cell or the host cell by a non-viral gene delivery method. Non-viral gene delivery methods, in some embodiments, deliver the genetic materials (including DNA, RNA and protein) into the target cell and express the targeting construct, the complementary strand oligonucleotides, and the nuclease, which allows the exogenous DNA of the targeting construct to be integrated into the host genome. In some embodiments, the non-viral method comprises transfection reagent (including nanoparticles) for DNA mRNA or protein, or electroporation.

### Methods of Treating Disease

Also provided herein are methods and compositions for treating disease, such as genetic disease. Genetic diseases are those that are caused by mutations in inherited DNA. In some embodiments, genetic diseases are caused by mutations in genomic DNA. Genetic mutations are known by those of skill in the art and include, single base-pair changes or point mutations, insertions, and deletions. In some embodiments, methods provided herein include a method of treating a genetic disease in a subject in need thereof, wherein the genetic disease results from a mutated gene having at least one changed nucleotide compared to a wild-type gene, wherein the method comprises contacting at least one cell of the subject with a composition comprising a targeting construct comprising a DNA sequence homologous to the wild-type gene and a targeting sequence, an oligonucleotide complementary to the targeting sequence, and a nuclease, wherein the targeting sequence is recognized by the nuclease such that the mutated gene, or fragment thereof, is replaced with the wild-type gene, or fragment thereof. Genetic diseases that are treated by methods disclosed herein include but are not limited to autosomal dominantly inherited diseases wherein at least the mutant allele is replaced with a correct copy of the gene provided by the donor DNA, preferably both the mutant and wildtype alleles are replaced with a correct copy of the gene provided by the donor DNA, or inherited and common diseases due to toxic gain-of-function, preferably said diseases comprising retinal dystrophy, preferably the retinal dystrophy is selected from retinitis pigmentosa, cone dystrophy or cone-rod dystrophy, macular degeneration e.g. Stargardt's Disease (ELOVL4), Von-Hippel Lindau, Retinoblastoma, RP4 (see RHO; OMIM: 180380), RP63 (see OMIM: 614494), CORD1 (cone rod dystrophy 1; see OMIM: 600624), CORD17 (cone rod dystrophy 17; see OMIM: 615163), BEST1 (bestrophin-1;Best disease; vitelliform macular dystrophy protein 2; see OMIM: 607854), OPA1 (OPA1 mitochondrial dynamin like GTPase; see OMIM: 605290), neuronal, hepatic diseases, lipofuscinoses (Batten's Disease and others), metabolic disorders, preferably for use in treating dominantly inherited ocular, e.g. retinal degeneration, preferably retinitis pigmentosa, neuronal and hepatic diseases. Retinal diseases that can be treated in the present invention are e.g. retinitis pigmentosa (due to mutations in RHO, AIPL1, IMPDH1, RDS, PDE6B or other genes), cone-rod dystrophy (CRX), Stargardt's Disease (ELOVL4), Von-Hippel Lindau and Retinoblastoma. In some embodiments, genetic diseases that are treated by the methods disclosed herein include recessive inherited diseases wherein at least one allele is replaced with a correct copy of the gene provided by the donor DNA or inherited and common diseases due to loss-of-function, preferably said diseases comprising haemophilia, diabetes, Lysosomal storage diseases comprising mucopolysaccharidoses (MPSI, MPSII, MPSIIIA, MPSIIIB, MPSIIIC, MPSIVA, MPSIVB, MPSVII), sphingolipidoses (Fabry's Disease, Gaucher Disease, Nieman-Pick Disease, GM1 Gangliosidosis), lipofuccinoses (Batten's Disease and others) and mucolipidoses; adenylosuccinate deficiency, hemophilia A and B, ALA dehydratase deficiency, adrenoleukodystrophy.

Methods of treating genetic disease disclosed herein preferably employ exogenous DNA sequences comprising at least a portion of a wild type DNA sequence that corresponds to the DNA sequence of mutated gene, so that in the method, the mutated DNA sequence is replaced with the wild type DNA sequence.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth. The term “genome editing” refers to a type of genetic engineering in which DNA is inserted, replaced, or removed from a target DNA, e.g. the genome of a cell, using one or more nucleases and/or nickases. The nucleases create specific double-strand breaks (DSBs) at desired locations in the genome, and harness the cell's endogenous mechanisms to repair the induced break by nonhomologous end joining (NHEJ). The nickases create specific single-strand breaks at desired locations in the genome. In one non-limiting example, two nickases can be used to create two single strand breaks on opposite strands of a target DNA, thereby generating a blunt or a sticky end. Any suitable nuclease can be introduced into a cell to induce genome editing of a target DNA sequence including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. The term “nonhomologous end joining” or “NHEJ” refers to a pathway that repairs double-strand DNA breaks in which the break ends are directly ligated without the need for a homologous template. The term “polynucleotide,” “oligonucleotide”, “nucleic acid”, “nucleotide” and “nucleic acid molecule” may be used interchangeably refers to deoxyribonucleic acids (DNA), ribonucleic acids (RNA) and polymers thereof in either single, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic, or derivatized nucleotide bases. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing non nucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. In some embodiments, a nucleic acid can comprise a mixture of DNA, RNA, and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with gene, cDNA, and mRNA encoded by a gene. The term “gene” or “nucleotide sequence encoding a polypeptide” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons). The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds. A “recombinant expression vector” is a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators), as well as those that confer certain binding affinity or antigenicity to the recombinant protein produced from the expression vector. The term “single nucleotide polymorphism” or “SNP” refers to a change of a single nucleotide with a polynucleotide, including within an allele. This can include the replacement of one nucleotide by another, as well as deletion or insertion of a single nucleotide. Most typically, SNPs are biallelic markers although tri- and tetra-allelic markers can also exist. By way of non-limiting example, a nucleic acid molecule comprising SNP A\C may include a C or A at the polymorphic position. The terms “subject,” “patient,” and “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance. As used herein, the term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. The term “treating” refers to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. The term “effective amount” or “sufficient amount” refers to the amount of an agent (e.g., DNA nuclease, etc.) that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

The term “pharmaceutically acceptable carrier” refers to a substance that aids the administration of an agent (e.g., DNA nuclease, etc.) to a cell, an organism, or a subject. “Pharmaceutically acceptable carrier” refers to a carrier or excipient that can be included in a composition or formulation and that causes no significant adverse toxicological effect on the patient. Non-limiting examples of pharmaceutically acceptable carrier include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical carriers are useful in the present invention.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

As used herein, the term “derivatives” also refers to longer or shorter polynucleotides/proteins and/or having e.g. a percentage of identity of at least 85%, 86%, 87%, 88%, 89%, 90%, 91%, 92%, 93%, 94%, 95%, 96%, 97%, 98%, more preferably of at least 99% with the sequences herein disclosed. In the present invention “at least 70% identity” means that the identity may be at least 70%, or 75%, or 80%, or 85% or 90% or 95% or 100% sequence identity to referred sequences. This applies to all the mentioned % of identity. Preferably, the % of identity relates to the full length of the referred sequence. The derivative of the invention also includes “functional mutants” of the polypeptides or polynucleotide, which are polypeptides or polynucleotide that may be generated by mutating one or more amino acids or nucleotide in their sequences and that maintain their activity. In the present invention “functional” is intended for example as “maintaining their activity”. Also within the scope of the subject invention are polynucleotides which have the same nucleotide sequences of a polynucleotide exemplified herein except for nucleotide substitutions, additions, or deletions within the sequence of the polynucleotide, as long as these variant polynucleotides retain substantially the same relevant functional activity as the polynucleotides specifically exemplified herein (e.g., they encode a protein having the same amino acid sequence or the same functional activity as encoded by the exemplified polynucleotide). Thus, the polynucleotides disclosed herein should be understood to include mutants, derivatived, variants and fragments, as discussed above, of the specifically exemplified sequences. The subject invention also contemplates those polynucleotide molecules having sequences which are sufficiently homologous with the polynucleotide sequences of the invention so as to permit hybridization with that sequence under standard stringent conditions and standard methods (Maniatis, T. et al, 1982).

### 2A Self-Cleaving Peptides

2A peptides, are 18-22 aa-long peptides which can induce the cleaving of recombinant proteins in the cell. 2A peptides are derived from the 2A region in the genome of virus.

Four members of 2A peptides family are frequently used in life science research. They are P2A, E2A, F2A and T2A. F2A is derived from foot-and-mouth disease virus 18; E2A is derived from equine rhinitis A virus; P2A is derived from porcine teschovirus-1 2A; T2A is derived from those a asigna virus 2. Said peptides preferably comprises or consist of the sequences below.

| Name | Sequence |
|---|---|
| T2A | (GSG) E G R G S L L T C G D V E E N P G |
| | P (SEQ ID NO: 3) |
| P2A | (GSG) A T N F S L L K Q A G D V E E N P |
| | G P (SEQ ID NO: 4) |
| E2A | (GSG) Q C T N Y A L L K L A G D V E S N |
| | P G P (SEQ ID NO: 35) |
| F2A | (GSG) V K Q T L N F D L L K L A G D V E |
| | S N P G P (SEQ ID NO: 36) |

Any ribosomal skipping sequence may be utilized within the meaning of the present invention. A preferred one is T2A or P2A.

### Splice Acceptor Sequences

RNA splicing is a form of RNA processing in which a newly made precursor messenger RNA (pre-mRNA) transcript is transformed into a mature messenger RNA (mRNA). During splicing, introns (non-coding regions) are removed and exons (coding regions) are joined together.

Within introns, a donor site (5′ end of the intron), a branch site (near the 3′ end of the intron) and an acceptor site (3′ end of the intron) are required for splicing. The splice donor site includes an almost invariant sequence GU at the 5′ end of the intron, within a larger, less highly conserved region. The splice acceptor site at the 3′ end of the intron terminates the intron with an almost invariant AG sequence. Upstream (5′-ward) from the AG there is a region high in pyrimidines (C and U), or polypyrimidine tract. Further upstream from the polypyrimidine tract is the branchpoint.

A “splice acceptor sequence” is a nucleotide sequence which can function as an acceptor site at the 3′ end of the intron. Consensus sequences and frequencies of human splice site regions are described in Ma, S. L., et al., 2015. PLoS One, 10(6), p.e0130729.

Suitably, the splice acceptor sequence may comprise the nucleotide sequence (Y)nNYAG, where n is 10-20, or a variant with at least 90% or at least 95% sequence identity. Suitably, the splice acceptor sequence may comprise the sequence (Y)nNCAG, where n is 10-20, or a variant with at least 90% or at least 95% sequence identity.

### Degradation Signal Sequence

The degradation signal sequence are preferably CL1, CL2, CL6, CL9, CL10, CL11, CL12, CL15, CL16, SL17, SMN, CIITA, ODc7, ecDHFR, PEST or a Mini ecDHFR sequence. Said sequences preferably comprises or consists of the sequences below or of the sequences encoding the sequence below.

| | | |
|---|---|---|
| CL2 | SLISLPLPTRVKFSSLLLIRIMKIITMTFPKKL | SEQ ID |
| | RS | NO: 37 |
| CL6 | FYYPIWFARVLLVHYQ | SEQ ID |
| | | NO: 38 |
| CL9 | SNPFSSLFGASLLIDSVSLKSNWDTSSSSCLIS | SEQ ID |
| | FFSSVMFSSTTRS | NO: 39 |
| CL10 | CRQRFSCHLTASYPQSTVTPFLAFLRRDFFFLR | SEQ ID |
| | HNSSAD | NO: 40 |
| CL11 | GAPHVVLFDFELRITNPLSHIQSVSLQITLIFC | SEQ ID |
| | SLPSLILSKFLQV | NO: 41 |
| CL12 | NTPLFSKSFSTTCGVAKKTLLLAQISSLFFLLL | SEQ ID |
| | SSNIAV | NO: 42 |
| CL15 | PTVKNSPKIFCLSSSPYLAFNLEYLSLRIFSTL | SEQ ID |
| | SKCSNTLLTSLS | NO: 43 |
| CL16 | SNQLKRLWLWLLEVRSFDRTLRRPWIHLPS | SEQ ID |
| | | NO: 44 |
| SL17 | SISFVIRSHASIRMGASNDFFHKLYFTKCLTSV | SEQ ID |
| | ILSKFLIHLLLRSTPRV | NO: 45 |
| ecDHFR | MISLIAALAVDYVIGMENAMPWNLPADLAWFKR | SEQ ID |
| | NTLNKPVIMGRHTWESIGRPLPGRKNI | NO: 46 |
| | ILSSQPSTDDRVTWVKSVDEAIAACGDVPEIMV | |
| | IGGGRVIEQFLPKAQKLYLTHIDAEVE | |
| | GDTHFPDYEPDDWESVFSEFHDADAQNSHSYCF | |
| | EILERR | |
| Mini | MISLIAALAVDYVIGMENAMPWNLPADLAWFKR | SEQ ID |
| ecDHFR | NTLNKPVIMGRHTWESIGRPLPGRKNI | NO: 47 |
| | ILSSQPSTDDRVTWVKSVDEAIAACGDVPEIMV | |
| | IGGGRVIEQFLP | |
| CIITA | RPGSTSPFAPSATDLPSMPEP | SEQ ID |
| | ALTSR | NO: 48 |
| SMN | YMSGYHTGYYMEMLA | SEQ ID |
| | | NO: 49 |
| ODC7 | MSCAQES | SEQ ID |
| | | NO: 50 |

For the sequences from CL2 to SL 17 see Gilon, T., Chomsky, O, and Kulka, R. G. (1998), Degradation signals for ubiquitin system proteolysis in Saccharomyces cerevisiae. The EMBO Journal, 17: 2759-2766, https://doi.org/i0.1093/emboi/17.10.2759 (herein incorporated by reference) while for sequences from ecDHFR to ODC7 see Tomnabene P, Trapani I, Centrulo M, Marrocco E, Minopoli R, Lupo M, Iodice C, Gesualdo C, Simonelli F, Surace E M, Auricchio A. Inclusion of a degron reduces levels of undesired inteins after AAV-mediated proteintrans-splicing in the retina. Mol Ther Methods Clin Dev. 2021 Oct. 19; 23:448-459. doi: 10.1016/j.omtm.2021.10.004. PMID: 34786437; PMCID: PMC8571531 (herein incorporated by reference).

In some embodiments, the degradation signal sequence is:
a C-terminal destabilizing peptide that shares structural similarities with misfolded proteins and is thus recognized by the ubiquitination system, ubiquitin, whose fusion at the N-terminal of a donor protein mediates both direct protein degradation or degradation via the N-end rule pathway, the N-terminal PB29 degron which is a 9 amino acid-long peptide which, similarly to the CL1 degron, is predicted to fold in structures that are recognized by enzymes of the ubiquitination pathway, artificial stop codons that cause the early termination of an mRNA, microRNA (miR) target sequences;
an N-degron and/or a C-degron. In some embodiments, the N-degron and/or the C-degron are independently a CL1, PB29, SMN, CIITA, or ODC degron. Such degradation signals are described in WO 2016/13932, which is incorporated by reference herein as it relates to degradation signals.

Another example of a degradation signal includes the E. coli dihydrofolate reductase (ecDHFR)-derived degron, as is described in WO 2020/079034 (incorporated by reference herein). Additional degradation signals include FKBP12 degradation domains (Banaszynski et al., Cell 126:995-1004, 2006), PEST degradation domains (Rechsteiner and Rogers, Trends Biochem Sci. 21:267-271, 1996), UbR tag ubiquitination signals (Chassin et al., Nat Commun. 10:2013, 2019), and destabilized mutations of human ELRBD (Miyazaki et al., J. Am. Chem. Soc., 134:3942-3945, 2012).

### Regulatory Elements

The construct of the invention may comprise one or more regulatory elements which may act pre- or post-transcriptionally. The one or more regulatory elements may facilitate expression in the cells of the invention.

A “regulatory element” is any nucleotide sequence which facilitates expression of a polypeptide, e.g. acts to increase expression of a transcript or to enhance mRNA stability. Suitable regulatory elements include for example promoters, enhancer elements, post-transcriptional regulatory elements and polyadenylation sites.

The subject invention also concerns constructs that can include regulatory elements that are functional in the intended host cell in which the vector comprising the construct is to be expressed. A person of ordinary skill in the art can select regulatory elements for use in appropriate host cells, for example, mammalian or human host cells. Regulatory elements include, for example, promoters, transcription termination sequences, translation termination sequences, enhancers, signal peptides, degradation signals and polyadenylation elements.

A construct of the invention may optionally contain a transcription termination sequence, a translation termination sequence, signal peptide sequence, internal ribosome entry sites (IRES), enhancer elements, and/or post-transcriptional regulatory elements such as the Woodchuck hepatitis virus (WHV) posttranscriptional regulatory element (WPRE). Transcription termination regions can typically be obtained from the 3′ untranslated region of a eukaryotic or viral gene sequence. Transcription termination sequences can be positioned downstream of a coding sequence to provide for efficient termination. In the system of the invention a transcription termination site is typically included.

### Post-Transcriptional Regulatory Elements

The nucleic acid constructs of the present invention may comprise post-transcriptional regulatory elements. Suitably, the protein-coding sequence is operably linked to one or more further post-transcriptional regulatory elements that may improve gene expression.

The construct of the present invention may comprise a Woodchuck Hepatitis Virus Post-transcriptional Regulatory Element (WPRE). Suitably, the OAT coding sequence is operably linked to a WPRE.

Suitable WPRE sequences will be well known to those of skill in the art (see, for example, Zufferey et al. (1999) Journal of Virology 73: 2886-2892; Zanta-Boussif et al. (2009) Gene Therapy 16: 605-619). Suitably, the WPRE is a wild-type WPRE or is a mutant WPRE. For example, the WPRE may be mutated to abrogate translation of the woodchuck hepatitis virus X protein (WHX), for example by mutating the WHX ORF translation start codon.

### Homology Arms

The nucleic acid constructs of the present invention may comprise one or more homology arms. For “homology arm” is intended a short sequence, typically of 2-20 bases, able to hybridize to at least one of the sequences flanking the targeting gene.

Typically, the nucleic acid construct comprises two homology arms, each one able to hybridize to each of the sequences flanking the targeting gene.

Homology arms are typically present when the genome editing strategy to be used is MMEJ.

### EXAMPLES

### Example 1

### Materials & Methods

Cas9 Effector Plasmids for gRNA Screening

gRNAs were designed manually considering the position of the targeted P23H mutation. Effector plasmids were generated following the protocol by Zhang's lab. In short, gRNAs were generated as a Fwd and Rev single-stranded DNA oligos, which were then phosphorylated and annealed using T4 Polynucleotide Kinase and T4 Ligation Buffer (NEB). Correct gRNA annealing was confirmed in a 50% acrylamide gel stained with SYBR Gold Nucleic Acid Stain (ThermoFisher). gRNAs were subsequently cloned into the px458 plasmid (#48138; Addgene) using BbsI sites.

### Generation of AAV Vector Plasmids

The plasmids used for AAV vector production derived from the pAAV2.1 plasmid that contains the ITRs of AAV serotype 2. Specifically, inventors used a pAAV2.1 plasmid generated by our group for a previous publication (4) The exact sequence is reported in the sequence file for reference.

### AAV Vector Production and Characterization

AAV vectors were produced by Innovavector SRL by triple transfection of HEK293 cells followed by two rounds of CsCl₂ purification. For each viral preparation, physical titers (GC/mL) were determined by averaging the titer achieved by dot-blot analysis and by PCR quantification using TaqMan (Applied Biosystems, Carlsbad, CA, USA). The probes used for dot-blot and PCR analyses were designed to anneal with the IRBP promoter for the pAAV2.1-IRBP-SpCas9-spA vector, and the bGHpA region for the donor DNA vectors. The length of probes varied between 200 and 700 bp(12).

### Culture and Transfection of HEK293 Cells

HEK293 cells were maintained in DMEM containing 10% fetal bovine serum (FBS) and 2 mM L-glutamine (Gibco, Thermo Fisher Scientific, Waltham, MA, USA). Cells were plated in 6-well plates (1*10⁶ cells/well) and transfected 16 hr later with the plasmids encoding for Cas9, a template plasmid that consists of Exon1, Intron1 and Exon2 of the human rhodopsin sequence driven by a cytomegalovirus promoter (CMV) and the different donor DNAs, using the calcium phosphate method (1 to 2 mg/1*10⁶ cells); medium was replaced 4 hr later. Maximum material transfected was 3 ug. In all cases, quantity of plasmid DNA was equilibrated between wells, using an empty vector when necessary.

### Histology and Light and Fluorescence Microscopy

To evaluate eGFP expression after HITI in vitro, HEK293 cells, plated in 6-wells at a density of 1*10⁶ were transfected as previously described. Seventy-two hours post-transfection, cells were analyzed under an Apotome (Carl Zeiss, Oberkochen, Germany) equipped with ZEN software (Carl Zeiss) and using appropriate excitation and detection settings for EGFP.

### Cytofluorimetric Analysis

HEK293 cells, plated in 6-well plates, were washed once with PBS, detached with trypsin 0.05% EDTA (Thermo Fisher Scientific, Waltham, MA USA), washed twice with PBS, and resuspended in sorting solution containing PBS, 5% FBS and 2.5 mM EDTA. Cells were analyzed on a BD FACS ARIA III (BD Biosciences, San Jose, CA, USA) equipped with BD FACSDiva software (BD Biosciences) using appropriate excitation and detection settings for EGFP. Thresholds for fluorescence detection were set on un-transfected cells, and a minimum of 10,000 cells/sample were analyzed. A minimum of 50,000 GFP+ were sorted and used for DNA extraction.

### Animal Models

Mice were housed at the TIGEM animal house (Pozzuoli, Italy) and maintained under a 12-hr light/dark cycle. The hRHO-P23H-TagRFP mice(8) (referred to as hRHO-P23H) mice were kindly provided by Prof Theodore Wensel. Mice were maintained by crossing homozygous females and males. Experimental heterozygous animals were generated by crossing homozygous P23H mice with C57BL/6 mice. The genotype of mice was confirmed by PCR analysis on genomic DNA (extracted from the mouse phalanx tip). Homozygous mice presented a 975 bp PCR product, while heterozygous mice presented a 975 bp and a 195 bp product. Wildtype mice presented only a 195 bp PCR product. The primers used for the PCR amplification are described in table 1 as follows:

TABLE 1
Description of primers used for PCR to detect
genotypes of mice described above.

| Primer Name | Sequence (5′-3′) | SEQ ID NO: |
|---|---|---|
| P23H-RFP_GENO | GTTCCGGAACTGCATGCTCACCAC | 51 |
| FP | | |
| P23H-RFP_GENO | CCCACCAGGAGCAGCGCC | 52 |
| RP | | |

Subretinal Injection of AAV Vectors in the hRHO-P23H Mice

This study was carried out in accordance with the Association for Research in Vision and Ophthalmology (ARVO) Statement for the Use of Animals in Ophthalmic and Vision Research and with the Italian Ministry of Health regulation for animal procedures (Ministry of Health authorization number: 252/2022-PR)

Mice (1 or 4 weeks old) were anesthetized with an intraperitoneal injection of 2 mL/100 g of body weight of ketamine/xylazine, then AAV2/8 vectors were delivered subretinally via a trans-scleral trans-choroidal approach, as described by Liang et al(13,14). Eyes were injected with 1 uL of vector solution. The AAV2/8 dose (GC/eye) was between 1,5*10⁹ and 2,5*10⁹ GC of each vector/eye; thus, co-injection resulted in a maximum of 3-5*10⁹GC/eye.

### Electrophysiological Recordings

For electroretinographic analyses, hRHO-P23H mice were dark-adapted for 3 hr. Mice were anesthetized and positioned in a stereotaxic apparatus, under dim red light. Pupils were dilated with a drop of 0.5% tropicamide (Visufarma, Rome, Italy), and body temperature was maintained at 37.5 degrees.

Light flashes were generated by a Ganzfeld stimulator (CSO, Costruzione Strumenti Oftalmici, Florence, Italy). The electrophysiological signals were recorded through gold-plate electrodes inserted under the lower eyelids in contact with the cornea. The electrodes in each eye were referred to a needle electrode inserted subcutaneously at the level of the corresponding frontal region. The different electrodes were connected to a two-channel amplifier. After completion of responses obtained in dark-adapted conditions (scotopic), the recording session continued with the purpose of dissecting the cone pathway mediating the light response (photopic). To minimize the noise, different responses evoked by light were averaged for each luminance step. The maximal scotopic response of rods and cones was measured in dark conditions (scotopic) with two flashes of 0.7 Hz and a light intensity of 20 cd s/m², photopic cone responses were isolated in light conditions with a continuous background white light of 50 cd s/m², with 10 flashes of 0.7 Hz and a light intensity of 20 cd s/m2.

### Retinal Cryosections and Fluorescence Imaging

To evaluate eGFP expression in the retina after HITI in histological sections, hRHO-P23H mice were injected subretinally with IRBP-Cas9 and donor DNA AAV vectors. One month later, mice were sacrificed, and eyes were fixed in 4% paraformaldehyde overnight and infiltrated with 30% sucrose overnight; the cornea and the lens were then dissected, and the eyecups were embedded in optimal cutting temperature compound (O.C.T. matrix; Kaltek, Padua, Italy). Ten-micrometer-thick serial retinal cryosections were cut along the horizontal meridian, progressively distributed on slides, and mounted with Vectashield with DAPI (Vector Lab, Peterborough, UK). Then, cryosections were analyzed under the confocal LSM-700 microscope (Carl Zeiss, Oberkochen, Germany), using appropriate excitation and detection settings for eGFP, RFP and DAPI respectively. For assessment of HITI efficiency in mouse retinal cryosections following AAV administration, the highest transduced area of three sections/eye was selected and acquired at 40 magnification and then analyzed using ImageJ software (http://rsbweb.nih.gov/ij/). A minimum of 500 PRs, identified by DAPI staining, were counted for each image manually and using the ImageJ plug-in ITCN. PRs with signal compatible with eGFP expression were unequivocally identified based on their shape as observed in z-stacks of the analyzed sections, as well as the presence of eGFP+ve outer segments.

### Optomotry

The visual acuity in mice was measured by the optomotor system (OptoMotry; www.cerebralmechanics.com). The mouse was positioned on a pedestal located in the center of a chamber consisting of four LCD monitors inwards facing. Upon some minutes of adaptation to the new environment, the test begins; a pattern of sinus stripes rotating clockwise and anti-clockwise appears on the monitor as determined randomly by the OptoMotry™ software (version VR 1.4.0). A response is considered positive when the mouse follow the direction of the gratings rotation.

### DNA Extraction

Samples (GFP+ sorted HEK293 cells, retinal tissue) were lysed in commercial lysis buffer (GeneArt™ Genomic Cleavage Detection Kit, Invitrogen, Carlsbad, California, United States) or conventional lysis buffer for DNA extraction from tissue (400 mM NaCl, 1% SDS, 20 mM TRIS-CL (pH 8.0), 5 mM EDTA (pH 8.0)) respectively. Lysis buffers were supplemented with proteinase K, which was inactivated after lysis for 15 minutes at 80 degrees. 50 to 200 ng of DNA were used for PCR amplification of the region comprising the Cas9 target site (the first intron of RHO) from the pCMV-hRHO (Exon1-Intron1-Exon2) plasmid or from the mouse genome, respectively. Primers used are shown in Table 2:

TABLE 2
hRHO-P23H-Indel primers produced a
461 bp PCR product.

| Primer name | Sequence (5′-3′) | SEQ ID NO: |
|---|---|---|
| Indel FW | TTGGAAGCCCGCATCTATC | 53 |
| Indel REV | GCCACATCCCTAAATGAGTC | 54 |

### HITI Junction Characterization

### Junction PCR Amplification

DNA extracted from retina was used for PCR amplification of HITI junctions. Both 5′ and 3′ junctions of integration were amplified. For the 5′ junction, inventors used a forward primer recognizing the region downstream of the first intron of the hRHO gene before the cut site and a reverse primer recognizing the Splice Acceptor Site-3×FLAG on the donor DNA. For the 3′ junction inventors designed a forward primer recognizing the bGH polyA sequence of the donor DNA, and a reverse primer recognizing the sequence within Intron 1 of Human RHO after the cut site.

TABLE 3
Primers used for amplifying the 5′ and 3′
junctions after HITI at the RHO locus
RNA extraction and hRHO expression

| | | SEQ ID |
|---|---|---|
| Primer name | Sequence (5′-3′) | NO: |
| Intron1 SeqFP2 | CTCTCAGCCCCTGTCCTCAG | 55 |
| NMD-HITI_5JnRP | CGTGGTCCTTATAGTCTACA | 56 |
| (short) | CCTGT | |
| 3′Int Junc FP_pA | GCCAGCCATCTGTTGTTTGC | 57 |
| 3′Int Jn RP | CCTGCCTCAGTTTTCCTCTC | 58 |
| (HITI) | TGTTA | |

Total RNA was extracted using the RNeasy MiniKit (QIAGEN) from both EGFP+/DsRed− and EGFP+/DsRed+ sorted HEK293 cells. RNA (5-15 ng) was used as a template for One-Step RT-qPCR (NEB, Massachusetts, USA) according to the manufacturer's instructions using the LightCycler 96 (Roche Molecular Systems, Inc.). Expression levels of hRHO were normalized vs. the corresponding housekeeping gene (ACTB). The relative quantification analysis was done using the 2(−ΔΔCt) method. The primers used for the real-time qPCR amplification are reported in table 4:

TABLE 4
primers used for the real-time qPCR
to detect hRHO Trancripts

| Primer name | Sequence (5′-3′) | SEQ ID NO: |
|---|---|---|
| RThRHO_Fw | ATCATGGTCATCGCTTTCCT | 59 |
| RThRHO_Rv | TCATGAAGATGGGACCGAAG | 60 |
| FwhActinQPCR | GGGAGAAGATGACCCAGATC | 61 |
| RvhActinQPCR | GGATAGCACAGCCTGGATAG | 8 |

### Results

### Optimizing the HITI Donor Results in Higher Efficiency In Vitro

To increase HITI efficiency inventors designed an optimized (optm.HITI) HITI construct by replacing the three tandem stop codons and the translation start sites (IRES/Kozak) with the CL1 degradation signal tagged with the 3×FLAG; this signal is fused to an active furin cleavage site for enhanced degradation of the truncated RHO protein. This was preceded by a splice acceptor sequence upstream for efficient splicing at the target site of the RHO locus. Upon cleavage by CRISPR-Cas9 and integration in the right orientation, the donor DNA will replace the endogenous RHO sequence in the genomic locus. Since the donor DNA is a promoter-less conding sequence (cds), it will get expressed only upon correct integration from the endogenous promoter (FIG. 2a). Apart from the RHO cds, the HITI donor also carries the eGFP cds so cells expressing eGFP, will allow us to determine the efficiency of integration. For this purpose, HEK 293 cells were transfected with i) Cas9 plasmid under the control of a CMV promoter ii) a template plasmid encoding the Human RHO Exon 1, Intron 1 and Exon 2 driven by a CMV promoter and lacking the poly-adenylation signal as described above and iii) the newly designed HITI donor plasmid consisting of the U6 expression cassette comprised of either the guide RNA (gRNA) to the first intron or a scrambled RNA sequence. Seventy-two hours after transfection, cells were imaged with a fluorescent microscope with appropriate excitation and emission filters (to detect the eGFP positive cells) and harvested for quantitative analysis of RHO transcripts by qPCR.

The inclusion of the CL1 degradation signal results in selective degradation of the 5′truncated endogenous RHO protein without affecting the production of full-length proteins (FIG. 2b). This degradation signal is further fused to P2A(12), a ribosomal skipping sequence, which will aid with the translation of the RHO coding sequence fused to an eGFP reporter protein via T2A and followed by WPRE and the bovine growth hormone (BGH) poly-A sequence. Inventors observed that the levels of hRHO transcripts were approximately 2,2-fold higher in cells that were transfected with the newly optimized HITI donor compared to cells that were transfected with the classical, 3×STOP donor (FIG. 2c) and this is correlated with a higher fluoresce intensity of cells transfected with the optimized HITI donor when compared to the cells with the classical, 3×STOP HITI donor.

### Evaluation of HITI Efficiency In Vivo

Inventors evaluated HITI efficiency in a recently described P23H knock-in mouse model of the autosomal dominant Retinitis Pigmentosa (RP4), wherein the endogenous RHO allele has been replaced by a red fluorescent protein tagged (RFP) human RHO harbouring the P23H mutation (hRHO-P23H-tagRFP)(11).

Inventors performed subretinal injections in hRHO-P23H-tagRFP heterozygous mice at 4-weeks of age, with two different AAV8 vectors, one encoding for the nuclease Sp.Cas9 under the control the photoreceptor specific promoter, Interphotoreceptor Retinoid-Binding Protein (IRBP), and a second AAV carrying the HITI donor DNA (carrying both RHO and GFP to label photoreceptors where integration occurred) at a dose of 1,5×10\hat{ }9 of each vector/eye. The contralateral eye served as control. Animals were sacrificed one-month post-treatment and eye were harvested for further analysis. HITI efficiency was evaluated by fluorescence microscopy imaging on retinal OCT sections by counting the number of GFP positive cells in the outer nuclear layer (ONL) over the DAPI stained nuclei. Inventors observed HITI efficiency up to 12±8% in the transduced area (n=4; FIG. 3a). Moreover, inventors also assessed HITI integration at the on-target site by performing HITI PCR amplification of both the 5′ and 3′ junctions between the HITI donor and the endogenous locus. Sanger sequencing analysis showed that HITI precisely occurred.

Prospectively, inventors plan to evaluate the improvement in the retinal phenotype (Electroretinogram ERG, visual acuity and morphology) at advanced timepoints and potential off-target editing events in both heterozygous and homozygous hRHO-P23H-tagRFP mice. Preliminary data in homozygous hRHO-P23H-tagRFP mice injected subretinally at p7 (1 week of age) with AAV-HITI gRNA (Optimized HITI) show an improved ERG response compared to AAV-HITI scRNA treated eyes 1-month post-treatment (FIG. 3b).

### Example 2

AAV-HITI Therapeutic Efficacy in hRHO-P23H-tagRFP Heterozygous Mice

Heterozygous hRHO-P23H-tagRFP mice were injected by subretinal injection at 4-weeks of age, with two different AAV8 vectors, one encoding for the nuclease Sp.Cas9 under the control of the photoreceptor specific promoter, Interphotoreceptor Retinoid-Binding Protein (IRBP), and a second AAV carrying one of the following donor DNA vectors: i. HITIgRNA ii. MMEJgRNA iii. scRNA at the dose of 1,5×10\hat{ }9 of each vector/eye. The construct used for HITIgRNA is p1501 (SEQ ID NO:32) and for respective scRNA is p1503 (SEQ ID NO:34); construct used for MMEJgRNA is p1515 (SEQ ID NO: 72) and for respective scRNA is p1519 (SEQ ID NO: 73). To evaluate the improvement in the retinal phenotype we performed electroretinographic (ERG) at different timepoints (p90-p150-p260-p360) and OCT analysis at p360 after AAVs delivery. In eyes injected with AAV-HITI gRNA and the AAV-MMEJgRNA expression cassette we found a significant improvement compared to AAV-scRNA injected eyes both by ERG (FIG. 4 A-B) and OCT analysis (FIG. 4C). Lastly, at p360 we also measured the visual acuity and found a significant improvement in AAV-HITIgRNA and AAV-MMEJ gRNA treated eyes (FIG. 4D).

### REFERENCES

1. Trapani I, Auricchio A. Seeing the Light after 25 Years of Retinal Gene Therapy. Trends Mol Med [Internet]. 2018 Aug. 1; 24(8):669-81. Available from: https://doi.org/10.1016/j.molmed.2018.06.006
2. Cong L, Ran F A, Cox D, Lin S, Barretto R, Habib N, et al. Multiplex Genome Engineering Using CRISPR/Cas Systems. Science (1979) [Internet]. 2013 Feb. 15; 339(6121):819-23. Available from: https://doi.org/i0.1126/science.1231143
3. Yanik M, Müller B, Song F, Gall J, Wagner F, Wende W, et al. In vivo genome editing as a potential treatment strategy for inherited retinal dystrophies. Prog Retin Eye Res [Internet]. 2017; 56:1-18. Available from: https://www.sciencedirect.com/science/article/pii/S1350946216300441
4. Hartong D T, Berson E L, Dryja T P. Retinitis pigmentosa. The Lancet [Internet]. 2006 Nov. 18; 368(9549):1795-809. Available from: https://doi.org/10.1016/S0140-6736(06)69740-7
5. Kaushal S, Khorana H G. Structure and Function in Rhodopsin. 7. Point Mutations Associated with Autosomal Dominant Retinitis Pigmentosa. Biochemistry [Internet]. 1994 May 1; 33(20):6121-8. Available from: https://doi.org/10.1021/bi00186a011
6. Suzuki K, Tsunekawa Y, Hernandez-Benitez R, Wu J, Zhu J, Kim E J, et al. In vivo genome editing via CRISPR/Cas9 mediated homology-independent targeted integration. Nature [Internet]. 2016; 540(7631):144-9. Available from: https://doi.org/10.1038/nature20565
7. Tornabene P, Ferla R, Llado-Santaeularia M, Centrulo M, Dell'Anno M, Esposito F, et al. Therapeutic homology-independent targeted integration in retina and liver. Nat Commun [Internet]. 2022; 13(1):1963. Available from: https://doi.org/10.1038/s41467-022-29550-8
8. Robichaux M A, Nguyen V, Chan F, Kailasam L, He F, Wilson J H, et al. Subcellular localization of mutant P23H rhodopsin in an RFP fusion knock-in mouse model of retinitis pigmentosa. Dis Model Mech [Internet]. 2022 May 6; 15(5):dmm049336. Available from: https://doi.org/10.1242/dmm.049336
9. Trapani I, Toriello E, de Simone S, Colella P, Iodice C, Polishchuk E v, et al. Improved dual AAV vectors with reduced expression of truncated proteins are safe and effective in the retina of a mouse model of Stargardt disease. Hum Mol Genet [Internet]. 2015 Dec. 1; 24(23):6811-25. Available from: https://doi.org/10.1093/hmg/ddv386
10. Gilon T, Chomsky O, Kulka R G. Degradation signals for ubiquitin system proteolysis in Saccharomyces cerevisiae. EMBO J [Internet]. 1998 May 15; 17(10):2759-66. Available from: https://doi.org/10.1093/emboj/17.10.2759
11. Thomas G. Furin at the cutting edge: from protein traffic to embryogenesis and disease. Nat Rev Mol Cell Biol [Internet]. 2002 October; 3(10):753-66. Available from: https://pubmed.ncbi.nlm.nih.gov/12360192
12. Doria M, Ferrara A, Auricchio A. AAV2/8 Vectors Purified from Culture Medium with a Simple and Rapid Protocol Transduce Murine Liver, Muscle, and Retina Efficiently. Hum Gene Ther Methods [Internet]. 2013 Sep. 12; 24(6):392-8. Available from: https://doi.org/10.1089/hgtb.2013.155
13. Liang F Q, Dejneka N S, Cohen D R, Krasnoperova N v., Lem J, Maguire A M, et al. AAV-Mediated Delivery of Ciliary Neurotrophic Factor Prolongs Photoreceptor Survival in the Rhodopsin Knockout Mouse. Molecular Therapy. 2001 Feb. 1; 3(2):241-8.
14. Liang F Q, Anand V, Maguire A M, Bennett J. Intraocular Delivery of Recombinant Virus. In: Rakoczy P E, editor. Vision Research Protocols [Internet]. Totowa, NJ: Humana Press; 2001. p. 125-39. Available from: https://doi.org/10.1385/1-59259-085-3:125
15. Ja-Hwan Seol, Eun Yong Shim and Sang Eun Lee. Microhomology-mediated end joining: Good, bad and ugly. Mutat Res. 2018 May; 809: 81-87.

1. A gene editing system comprising:
a) a donor nucleic acid comprising:
a degradation signal sequence,
an enzymatic cleavage site,
a ribosomal skipping sequence,
an exogenous DNA sequence,
wherein said donor nucleic acid is flanked at 5′ and 3′ by inverted targeting sequences;
b) an oligonucleotide complementary to the targeting sequence and
c) a nuclease that recognizes the targeting sequence.

2. The gene editing system of claim 1, wherein said donor nucleic acid elements are in the 5′-3′ order as listed.

3. The gene editing system of claim 1, wherein said donor nucleic acid further comprises a splice acceptor sequence.

4. The gene editing system of claim 1 wherein the degradation signal sequence is selected from the group consisting of: CL1, CL2, CL6, CL9, CL10, CL11, CL12, CL15, CL16, SL17, SMN, CIITA, ODc7, ecDHFR, PEST and a Mini ecDHFR sequence.

5. The gene editing system of claim 1, wherein the degradation signal sequence is CL1 and/or wherein the enzymatic cleavage site is a furin cleavage site and/or wherein the ribosomal skipping sequence is a ribosomal skipping sequence from Porcine Tescho virus-1 2A (P2A).

6. The gene editing system of claim 1 wherein the targeting sequence is a sequence comprised in rhodopsin (Rho) gene
and/or wherein the exogenous DNA sequence comprises a coding sequence of a therapeutic protein,
and/or wherein the targeting sequence is a guide RNA (gRNA) target site,
and/or wherein said oligonucleotide complementary to the targeting sequence is a guide RNA that hybridizes to a targeting sequence of a gene or to its complementary strand,
and/or said oligonucleotide complementary to the targeting sequence is under the control of a promoter,
and/or wherein the inverted targeting sequences is an inverted sequence with respect to a target sequence and/or comprises a PAM sequence.

7. The gene editing system of claim 1 wherein said donor nucleic acid further comprises one or more of:
a linker;
a further ribosomal skipping sequence;
a post-transcriptional regulatory element;
a transcription termination sequence.

8. The gene editing system of claim 1 wherein said donor nucleic acid comprises in a 5′-3′ order:
an inverted targeting sequence with its protospacer-adjacent motif (PAM) sequence;
a splice acceptor sequence
a degradation signal sequence, preferably CL1 sequence,
an enzymatic cleavage site, preferably a furin cleavage site,
a ribosomal skipping sequence, preferably a P2A sequence,
an exogenous DNA sequence, preferably one or more rhodopsin exons,
a further ribosomal skipping sequence, preferably T2A,
a transcription termination sequence, and
a further inverted targeting sequence with its protospacer-adjacent motif (PAM) sequence.

9. The gene editing system of claim 1 wherein said donor nucleic acid further comprises at least an homology arm.

10. The gene editing system according to claim 9 wherein said donor nucleic acid comprises, in a 5′-3′ order:
an inverted targeting sequence with its protospacer-adjacent motif (PAM) sequence;
a first homology arm
a splice acceptor sequence
a degradation signal sequence, preferably CL1 sequence,
an enzymatic cleavage site, preferably a furin cleavage site,
a ribosomal skipping sequence, preferably a P2A sequence,
an exogenous DNA sequence, preferably one or more rhodopsin exons,
a further ribosomal skipping sequence, preferably T2A,
a transcription termination sequence,
a second homology arm and
a further inverted targeting sequence with its protospacer-adjacent motif (PAM) sequence.

11. The gene editing system according to claim 1 wherein:
a) the ribosomal skipping sequence comprises or has essentially a sequence having at least 80% of identity to SEQ ID NO: 1 (GCCACCAACTTCTCCCTGCTGAAGCAGGCCGGCGACGTGGAGGAGAACCCCGG CCCC) or to SEQ ID NO: 2 (GGAAGCGGAGAGGGCAGAGGAAGTCTGCTAACATGCGGTGACGTCGAGGAGAA TCCTGGACCT)
or to a sequence encoding for SEQ ID NO: 3 (GSG) E G R G S L L T C G D V E E N P G P or to a sequence encoding for SEQ ID NO: 4 (GSG) A T N F S L L K Q A G D V E E N P G P or functional fragments thereof and/or
b) the inverted targeting sequence comprises or has essentially a sequence having at least 95% of identity to SEQ ID NO: 5 (ACACCAGGAGACTTGGAACG) or functional fragments thereof and optionally comprises the SpCas9 PAM sequence (CGG) and/or
c) the guide RNA comprises or has essentially a sequence having at least 95% of identity to SEQ ID NO: 5 (ACACCAGGAGACTTGGAACG), or functional fragments thereof
d) the oligonucleotide complementary to the targeting sequence comprises or has essentially a sequence having at least 95% of identity to SEQ ID NO: 5 (ACACCAGGAGACTTGGAACG) or functional fragments thereof and/or
e) the degradation signal sequence comprises or has essentially a sequence having at least 80% of identity to SEQ ID NO: 6 (gcctgcaagaactggttcagcagcctgagccacttcgtgatccacctg) and/or
f) the enzymatic cleavage site comprises or has essentially a sequence having at least 95% of identity to SEQ ID NO: 7 (CGAAAAAGAAGA) and/or
g) the linker comprises or has essentially a sequence having at least 95% of identity to ggaagcgga and/or
h) the splice acceptor sequence comprises or has essentially a sequence having at least 80% of identity to SEQ ID NO: 9 (GATAGGCACCTATTGGTCTTACTGACATCCACTTTGCCTTTCTCTCCACAGGTGT) and/or
i) the exogenous DNA sequence comprises or has essentially a sequence having at least 80% of identity to at least one of the following sequences: SEQ ID NO: 10
(ATGAATGGCACAGAAGGCCCTAACTTCTACGTGCCCTTCTCCAATGCGACGGGT
GTGGTACGCAGCCCCTTCGAGTACCCACAGTACTACCTGGCTGAGCCATGGCAG
TTCTCCATGCTGGCCGCCTACATGTTTCTGCTGATCGTGCTGGGCTTCCCCATCAA
CTTCCTCACGCTCTACGTCACCGTCCAGCACAAGAAGCTGCGCACGCCTCTCAAC
TACATCCTGCTCAACCTAGCCGTGGCTGACCTCTTCATGGTCCTAGGTGGCTTCA
CCAGCACCCTCTACACCTCTCTGCATGGATACTTCGTCTTCGGGCCCACAGGATG
CAATTTGGAGGGCTTCTTTGCCACCCTGGGCG),
SEQ ID NO: 11
(GTGAAATTGCCCTGTGGTCCTTGGTGGTCCTGGCCATCGAGCGGTACGTGGTGG
TGTGTAAGCCCATGAGCAACTTCCGCTTCGGGGAGAACCATGCCATCATGGGCG
TTGCCTTCACCTGGGTCATGGCGCTGGCCTGCGCCGCACCCCCACTCGCCGGCTG
GTCCAG);
SEQ ID NO: 12
(GTACATCCCCGAGGGCCTGCAGTGCTCGTGTGGAATCGACTACTACACGCTCAA
GCCGGAGGTCAACAACGAGTCTTTTGTCATCTACATGTTCGTGGTCCACTTCACC
ATCCCCATGATTATCATCTTTTTCTGCTATGGGCAGCTCGTCTTCACCGTCAAGGA
G);
SEQ ID NO: 13
(GCCGCTGCCCAGCAGCAGGAGTCAGCCACCACACAGAAGGCAGAGAAGGAGGT
CACCCGCATGGTCATCATCATGGTCATCGCTTTCCTGATCTGCTGGGTGCCCTAC
GCCAGCGTGGCATTCTACATCTTCACCCACCAGGGCTCCAACTTCGGTCCCATCT
TCATGACCATCCCAGCGTTCTTTGCCAAGAGCGCCGCCATCTACAACCCTGTCAT
CTATATCATGATGAACAAGCAG);
SEQ ID NO: 14
(TTCCGGAACTGCATGCTCACCACCATCTGCTGCGGCAAGAACCCACTGGGTGAC
GATGAGGCCTCTGCTACCGTGTCCAAGACGGAGACGAGCCAGGTGGCACCAGCA)
and/or
j) the woodchuck hepatitis virus post transcriptional
regulatory element (wpre) comprises or has essentially a
sequence having at least 80% of identity to SEQ ID NO: 15
(Taagcttggatccaatcaacctctggattacaaaatttgtgaaagattgactggtat
tcttaactatgttgctccttttacgctatgtggatacgctgctttaatgcctttgtat
catgctattgcttcccgtatggctttcattttctcctccttgtataaatcctggttgc
tgtctctttatgaggagttgtggcccgttgtcaggcaacgtggcgtggtgtgcactgt
gtttgctgacgcaacccccactggttggggcattgccaccacctgtcagctcctttcc
gggactttcgctttccccctccctattgccacggeggaactcategccgcctgccttg
cccgctgctggacaggggctcggctgttgggcactgacaattccgtggtgttgtcggg
gaaatcatcgtcctttccttggctgctcgcctgtgttgccacctggattctgcgcggg
acgtccttctgctacgtcccttcggccctcaatccagcggaccttccttcccgcggcc
tgctgccggctctgcggcctcttccgcgtcttcg)
and/or
k) the bovine Growth Hormone Poly-Adenylation Signal
(BGH pA) comprises or has essentially a sequence having
at least 80% of identity to SEQ ID NO: 16
(GCCTCGACTGTGCCTTCTAGTTGCCAGCCATCTGTTGTTTGCCCCTCCCCCGTGC
CTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGGGGGTGGGG
CAGGACAGCAAGGGGGAGGATTGGGAAGACAATAGCAGGCATGCTGGGGA)
and/or
l) the homology arm comprises or has essentially a sequence
having at least 80% of identity to SEQ ID NO: 66
(ctccctgccgg) or SEQ ID NO: 68 (tgagaaccgc)

12. The gene editing system according to claim 1 wherein the nuclease is selected from: a CRISPR nuclease, a TALEN, a DNA-guided nuclease, a meganuclease, and a Zinc Finger Nuclease.

13. A vector that comprises the gene editing system according to claim 1 or the donor nucleic acid and/or the oligonucleotide complementary to the targeting sequence and/or a nuclease that recognizes the targeting sequence.

14. The vector according to claim 13 comprising a construct comprising said donor nucleic acid and complementary oligonucleotide, wherein said construct comprises or has essentially a sequence having at least 80 or at least 85 or at least 90 or at least 95% of identity to SEQ ID NO. 32 or SEQ ID NO. 72.

15. A host cell comprising the gene editing system according to claim 1.

16. A viral particle that comprises the gene editing system according to claim 1.

17. A pharmaceutical composition that comprises gene editing system according to claim 1, and a pharmaceutically acceptable carrier.

18. A method of treatment of a genetic disease comprising using the gene editing system according to claim 1.

19. A method of treating a genetic disease and/or autosomal dominantly inherited diseases wherein at least the mutant allele is replaced with a correct copy of the gene provided by the donor DNA, or treating inherited and common diseases due to toxic gain-of-function comprising using the gene editing system according to claim 1.

20. A method of treating recessive inherited diseases wherein at least one allele is replaced with a correct copy of the gene provided by the donor DNA or treating inherited and common diseases due to loss-of-function comprising using the gene editing system according to claim 1.
